(19) United States
(12) Patent Application Publication
(10) Pub. No.: US 2008/0254031 A1
(43) Pub. Date: Oct. 16, 2008

(54) Tumor Specific Genes and Variant Rnas and Uses Thereof as Targets for Cancer Therapy and Diagnosis

(75) Inventors: Kevin McGowan, N. Potomac, MD (US); Vinayaka Kotraiah, Germantown, MD (US); Michael Brenner, Fredrick, MD (US); Richard Einstein, Gaithersburg, MD (US); Laurent Bracco, La Garenne Colomba (FR)

Correspondence Address: OCCHIUTI ROHLICEK & TSAO, LLP, 10 FAWCETT STREET, CAMBRIDGE, MA, 02138, US

(73) Assignee: EXONHIT THERAPEUTICS SA, Paris (FR)

(21) Appl. No.: 11/575,032

(22) PCT Filed: Sep. 7, 2005
(86) PCT No.: PCT/IB05/02934

§ 371 Date: Jan. 10, 2008

Related U.S. Application Data
(60) Provisional application No. 60/608,076, filed on Sep. 9, 2004.

(51) Int. Cl. A61K 39/395; C07H 21/04; C07K 14/47; A61K 39/00; C12Q 1/68; G01N 33/574; A61K 38/02; A61P 35/00; G01N 33/566; G01N 33/53; C07K 16/32; C07K 19/00; A61K 38/47; A61K 31/7052

(52) U.S. Cl. 424/138.1; 536/23.5; 536/24.33; 530/300; 530/350; 424/185.1; 435/6; 435/7.23; 435/7.92; 435/7.93; 530/387.7; 530/391.3; 424/94.61; 514/44; 514/2; 436/501

(57) ABSTRACT

Genes and variant RNAs that are differentially expressed in human colon tumor tissues compared with normal colon tissue and the corresponding proteins are identified. These genes and the corresponding antigens are suitable targets for the treatment, diagnosis or prophylaxis of colon cancer.

### FIELD OF THE INVENTION

The present invention relates to the identification of nucleic acid sequences that correspond to alternatively spliced events in genes expressed from colon cancer cells. These genes or their corresponding proteins represent novel targets for the treatment, prevention and/or diagnosis of cancers wherein these genes are differentially regulated and/or spliced, particularly in colon cancer. The present invention also relates to compounds that specifically bind or modulate said targets, including antibodies, compositions comprising the same and their uses. The invention also provides novel products or constructs, including primers, probes, cells, chips and the like, for use in diagnostic or pharmacogenomic methods. The invention is suited for use in mammalians, particularly human subjects.

### BACKGROUND OF THE INVENTION

Genetic detection of human disease states is a rapidly developing field (Taparowsky et al., 1982; Slamon et al., 1989; Sidransky et al., 1992; Miki et al., 1994; Dong et al., 1995; Morahan et al., 1996; Lifton, 1996; Barinaga, 1996). However, some problems exist with this approach. A number of known genetic lesions merely predispose an individual to the development of specific disease states. Individuals carrying the genetic lesion may not develop the disease state, while other individuals may develop the disease state without possessing a particular genetic lesion. In human cancers, genetic defects may potentially occur in a large number of known tumor suppresser genes and proto-oncogenes.

Genetic detection of cancer has a long history. Some of the earliest genetic lesions shown to predispose to cancer were transforming point mutations in the ras oncogenes (Taparowsky et al., 1982). Transforming ras point mutations may be detected in the stool of individuals with benign and malignant colorectal tumors (Sidransky et al., 1992). However, only 50% of such tumors contained a ras mutation (Sidransky et al., 1992). Similar results have been obtained with amplification of HER-2/neu in breast and colon cancer (Slamon et al., 1989), deletion and mutation of p53 in bladder cancer (Sidransky et al., 1991), deletion of DCC in colorectal cancer (Fearon et al., 1990) and mutation of BRCA1 in breast and colon cancer (Miki et al., 1994).

None of these genetic lesions are capable of predicting a majority of individuals with cancer and most require direct sampling of a suspected tumor, and make screening difficult. Further, none of the markers described above are capable of distinguishing between metastatic and non-metastatic forms of cancer. In effective management of cancer patients, identification of those individuals whose tumors have already metastasized or are likely to metastasize is critical. Because metastatic cancer kills 560,000 people in the U.S. each year (ACS home page), identification of markers for metastatic colon cancer would be an important advance.

Colon cancer is one of the most prevalent cancers affecting more than 147,500 new patients yearly in the US. Ten million FOBT tests are used as a screening test annually in the US. The other tests for colon cancer are invasive tests with the exception of predisposition tests. A non-invasive test that could detect early-stage CRC would represent a significant improvement from current screening devices. Additionally, a blood diagnostic test could also be utilized as a monitoring test to check for the recurrence of the disease, thereby increasing the number of tests performed yearly.

Colorectal cancer (CRC) (Midgley and Kerr, 1999) is a leading cause of morbidity and mortality with about 300,000 new cases and 200,000 deaths in Europe and the USA each year. It is the third most common cancer in men and women (American Cancer Society, 2002). In the USA, mortality rates have steadily declined among women since about 1950 and among men since approximately 1985 (American Cancer Society, 2002). The estimated five-year survival rate for patients diagnosed with early stage CRC is nearly 90%. Thus, early detection is a key component for managing the disease. Despite the proven effectiveness and availability of various colorectal cancer screening tests, many adults aged 50 or older are not regularly screened. Prevalence rates are especially low among individuals who are 50-64 years old, have lower incomes little or no health care coverage, and fewer years of education. As a consequence, only 37% of cases are diagnosed when the disease is still localised. Later diagnosis results in a substantially lower 5-year relative survival rate (64.4% for locally spread cancers, 8.3% for metastasised cancers) than would occur if patients were diagnosed when disease is still localized. Additionally, there is increased risk for recurrence of the disease in late-stage patients following surgery, as nearly 50% of patients believed to be cured by surgery will relapse and succumb to the disease.

Most colorectal cancers arise in the sigmoid colon (the portion just above the rectum). They usually start in the innermost layer and can grow through some or all of the several tissue layers that make up the colon and rectum. Most large bowel cancers arise within pre-existing adenomatous polyps or adenomas. These lesions are common. Necropsies have shown a prevalence of 35% in Europe and the USA with lower rates (10-15%) in Asia and Africa. Adenomas are classified by histological architecture as tubular, tubulovillous or villous. Villous change is associated with a higher malignant potential, as are large (up to 25% of adenomas are >1 cm in diameter) and high-grade epithelial dysplasia (severe dysplasia is found in 5-10% of adenomatous polyps). It is estimated that approximately 5% of adenomatous polyps will become malignant, a transformation that may take 5 to 10 years.

There is growing recognition that this adenoma-carcinoma sequence results from the interplay of environmental and genetic components. Genetic mutations are either inherited as germline defects or arise in somatic cells, secondary to environmental insults. There are two main inherited predisposition syndromes: Familial Adenomatous Polyposis (FAP) and Hereditary Non Polyposis Colorectal Cancer (HNPCC). Theses inherited predispositions for colorectal cancer share the same random pathway of progression form adenoma to carcinoma with the sporadic form, even if the progression rate and timescale of occurrence differ.

A multi-step model of progression of sporadic colorectal cancer has been proposed by Vogelstein et al (1988) which hypothesise that a combination of four or five mutations must accumulate in the cell, including activation of oncogenes and inactivation of tumour suppressor genes, to undergo full malignant transformation. This is consistent with the observation that colorectal cancers occur predominantly in the elderly. If one or more defects are present at birth, less additional mutations will be necessary to occur and the disease will appear earlier.

The majority of CRCs are treated through surgical removal of the bowel. Traditionally, this has involved open resection using a laparotomy to enable both resection of the primary tumour with sufficient excision margins and an adequate, systematic lymphadenectomy. Additionally, for rectal cancer, a total mesorectal excision is performed to reduce the probability of local recurrence (Vogelstein et al., 1988). Excision of the tumour is the primary treatment for new CRC cases with potential for cure (80%). In the remaining 20%, the disease is too far advanced at presentation (either locally or at distant sites) for any curative intervention. These patients also frequently undergo surgery for palliation, where optimising quality of life is the main objective of treatment. In this setting, chemotherapy has an established role in improving survival and palliating symptoms. In addition, approximately 50% of those patients initially believed to be cured by surgery, subsequently relapse and die of their disease. Adjuvant chemotherapy administered for six months after surgery for Dukes C colon cancer improves absolute survival by 5-10% (Midgley and Kerr, 1999).

| Classification | Dukes' A | Dukes' B | Dukes' C | Dukes' D |
|---|---|---|---|---|
| Cancer | Cancer defined to | Cancer may extend | Cancer may extend | Metastatic disease. |
| Progression | most superficial | completely | completely | The cnacer has |
| | cell layers of colon | through wall of | through wall of | spread to distant |
| | or rectum | colon or rectum, | colon or rectum, | organs, such as the |
| | | no lymph node | and has spread to | liver. |
| | | involvement | lymph nodes | |
| Standard | Surgery | Surgery | Surgery and | Surgery and |
| Treatment | | | Chemotherapy | Chemotherapy |
| | | | (possibly radiation | (possibly radiation |
| | | | for rectal cancer | for rectal cancer |
| Estimated 5-Year | 95% | 80% | 50% | 5% |
| survival rate | | | | |

| | | |
|---|---|---|
| Percent diagnosed | 37% | 63% |
| at stage | | |

Table taken from the American Cancer Society, Cancer facts and figures, 2002

Assuming correct and early diagnosis, approximately 90% of all colorectal cancer cases are thought to be curable (American Cancer Society, 2002). To improve the likelihood for early detection various screening programs have been recommended for both the general and high-risk populations (Midgley and Kerr, 1999). The recommendation of the American Cancer Society for CRC screening is as follows (Smith et al., 2001):
1 low risk patients;
   an annual faecal occult blood test (FOBT) and sigmoidoscopy (FSIG) every five years starting at age 50 or
   colonoscopy and digital rectal examination (DRE) every 10 years or
   double-contrast barium enema and digital rectal examination every 5-10 years
2 High risk patients (family history of CRC or polyps or chronic inflammatory bowel disease) screening should begin earlier and be more frequent.

The specifics of each test are described below. Procedures such as sigmoidoscopy and colonoscopy are utilized as both screening tests and diagnostic tests. For example, colonoscopy is performed routinely on healthy patients over age 50 as a screening test for early detection of colon cancer. However, it is also used for diagnostic purposes when patients present with clear symptoms of a bowel disorder such as blood in stool, positive FOBT test, and/or excessive cramping. It is important to note that positive FOBT can result from a number of factors outside of colon cancer.
1 Faecal Occult Blood Test (FOBT). The FOBT detects the presence of blood in stool, derived from colorectal cancer or large (>2 cm) polyps. Individual receive a kit to take home along with dietary instructions. FOBT consists of six small stool samples each taken from three consecutive bowel movements. Upon completing the test, patients return the kit to the physician for evaluation. The test is not specific for colon cancer as other conditions such as ulcers, colitis, hemorrhoids also bleed. A positive FOBT will require further diagnostic work-up such as colonoscopy or double-contrast barium enema. Several studies have proven that the regular use of this screening method saves life and can reduce the incidence of colorectal cancer by diagnosing and removing earlier precancerous lesions (Mandel et al., 1993; Mandel et al., 2000; Kronborg et al., 1996; Hardcastle et al., 1996).
2 Flexible Sigmoidoscopy (FSIG). A slender, flexible, hollow, lighted tube is inserted through the rectum into the colon to search for cancer or polyps. The sigmoidoscope is around 2 feet long and, at its maximum of insertion, can only reach about half of the colon. If there is a polyp or tumour present, the patient must be referred for colonoscopy so that the entire colon can be examined. The advantage of FSIG over FOBT is that it allows the examiner to visualize the distal bowel directly and it has higher sensitivity and specificity for both adenocarcinomas and polyps. The limitation of the FSIG is that the instrument cannot visualize the entire length of the colon. Removal of polyps is rare during this procedure as the bowel is not sufficiently prepared for surgery. Additionally, the presence of polyps in the sigmoid colon is usually an indication of polyps or cancer in the proximal bowel.
3 Colonoscopy. This procedure allows for direct visual examination of the entire colon as well as surgical removal of the polyps. The procedure is somewhat uncomfortable and may not always be covered under health care plans.
4 Barium Enema with air contrast (Double-contrast barium enema). Barium sulphate is introduced into the colon and allowed to spread to partially fill and open up the colon. The colon is then filled with air so that it can expand and increase the quality of x-rays that are taken. It is superior to FSIG in that it detects small polyps (<9 mm) with accuracy and examines the entire bowel. The test can be uncomfortable especially when done as a double-contrast procedure in which air is introduced into the bowel for contrast.

There is a need in the art for genetic markers and targets of colon cancers, allowing the design of specific, reliable and sensitive diagnostic and therapeutic approaches of these diseases.

### SUMMARY OF THE INVENTION

The present invention relates to the identification of novel nucleic acid and amino acid sequences that are characteristic of colon cancer cells or tissues, and which represent targets for therapy or diagnosis of such a condition in a subject.

The invention more specifically discloses 60 specific, isolated nucleic acid molecules that encode expression sequences found to be differentially expressed in colon cancer. Of these, 51 are expressed sequence tags that are differentially spliced and correspond to SEQ ID NOS 1-44, 52, 56, 62, 73, 79, 85, and 91. In addition, 9 specific isoforms of known genes have been identified corresponding to SEQ ID NOS. 47, 53, 57, 65, 70, 76, 82, 88, and 96. These novel sequences were found to be differentially expressed between normal colon and colon cancer. The expressed sequence tag represent novel exons that are alternatively spliced in colon cancer, and as such, directly identify distinct isoforms. These sequences and molecules represent targets and valuable information to develop methods and materials for the detection, diagnosis, and treatment of colon cancer. Furthermore, since deregulations of RNA splicing have been observed in distinct types of cancers, and because said deregulations constitute a mechanism by which response to chemotherapy may be altered, the presently characterized nucleic acids and polypeptides may also represent target molecules suitable for other cancers as well.

It is thus an object of the invention to provide methods and materials for treatment and diagnosis of cancer, particularly colon cancer.

In particular, an object of this invention resides in nucleic acids and amino acids, which are differentially regulated in colon cancer cells. More particularly, an object of this invention resides in isolated nucleic acids that are expressed by human cancer cells, particularly colon cancer cells, selected from the group consisting of:
(i) nucleic acids comprising a sequence contained in SEQ ID NOS. 1-44, 47, 52, 53, 56, 57, 62, 65, 70, 73, 74, 76, 79, 82, 85, 88, 91, and 96;
(ii) a nucleic acid having a sequence that is at least 70% identical to the sequence of (i) when aligned without allowing for gaps;
(iii) nucleic acids having a sequence complementary to (i) or (ii); and
(iv) fragments of (i), (ii) or (iii) having a size of at least 20 nucleotides in length.

A further object of this invention resides in any polypeptide (or antigen) encoded by a nucleic acid as defined above. More particularly, the invention relates to polypeptides expressed by human cancer cells, selected from the group consisting of:
(i) the polypeptide encoded by a nucleic acid sequence having at least 90% sequence identity in SEQ ID NOS. 1-44, 47, 52, 53, 56, 57, 62, 65, 70, 73, 74, 76, 79, 82, 85, 88, 91, and 96; or a sequence complementary thereto; and
(ii) the polypeptide comprising an amino acid sequence having at least 90% sequence identity in SEQ ID NOS. 48, 49, 54, 55, 58, 59, 66, 67, 71, 72, 75, 77, 78, 83, 84, 89, 90, 97, 98 and 99; and
(iii) an antigenic fragment of (i) or (ii).

Another object of the invention is to provide novel methods for diagnosis or detection of cancer, particularly colon cancer by using ligands (e.g., monoclonal antibodies, probes, etc.) which specifically bind to a target molecule (i.e., polypeptide or nucleic acid) as defined above. Such methods may be used to detect whether a subject has or is at (increased) risk of developing a cancer, particularly colon cancer or, for instance, whether a treatment regimen is efficient.

In this respect, a particular object of the invention resides in methods of detecting persons having, or at (increased) risk of developing a cancer, particularly colon cancer, by use of labeled nucleic acid probes that hybridize to a target gene or nucleic acid as defined in the present application.

According to an other embodiment of the invention, the methods of detecting persons having, or at (increased) risk of developing cancer, particularly colon cancer, use a (labeled) antibody or fragment/derivative thereof that specifically binds a target polypeptide as defined in the present application.

A further object of this invention relates to diagnostic test kits for the detection of persons having or at (increased) risk of developing cancer, particularly colon cancer, that comprise a ligand that specifically binds to a target molecule as defined above and, optionally, a detectable label, e.g. indicator enzymes, a radiolabels, fluorophores, or paramagnetic particles. In a particular embodiment, the ligand comprises nucleic acid primers or probes specific for target genes or nucleic acids as described above, or an antibody or a derivative thereof, specific for a target polypeptide as described in this application.

A further aspect of this invention resides in the development of novel therapies for treatment of cancer, particularly colon cancer, involving the administration of an inhibitor of a target molecule as defined in the present application. In a particular embodiment, the method comprises administering an inhibitory nucleic acid (e.g., anti-sense oligonucleotide, ribozyme, iRNA, siRNA or a DNA encoding the same) corresponding to (i.e., complementary and specific for) a target nucleic acid as described herein, thereby inhibiting (e.g., reducing) expression or translation thereof. In an other embodiment, the method comprises administering an antibody that specifically binds a target polypeptide as described herein.

A further object of this invention relates to methods of treating cancer, particularly colon cancer, in a subject, comprising the administration of a polypeptide antigen as described herein, alone or in combination with adjuvants that elicit an antigen-specific cytotoxic T-cell lymphocyte response against cancer cells that express such antigen.

It is another object of this invention to provide methods for selecting, identifying, screening, characterizing or optimizing biologically active compounds, comprising a determination of whether a candidate compound binds, preferably selectively, an antigen or a polynucleotide as disclosed in the present application. Such compounds represent drug candidates or leads for treating cancer diseases, particularly colon cancer.

A further object of this invention resides in a method of producing or selecting ligands that bind a target molecule as described herein, comprising contacting a candidate compound with a target molecule and determining the ability of such compound to bind said target. The method is particularly suited for selecting or producing ligands of an extra-cellular domain of a polypeptide (antigen) encoded by a gene or exon expressed by certain cancers.

It is another object of the invention to identify genes that are expressed in altered forms in colon cancer cells. These forms represent splice variants of the gene, where the Expressed Sequence Tag either 1) indicates the splice event occurring within the gene, or 2) points to a gene that is actively spliced to produce different gene products. These different splice variants or isoforms can be targets for therapeutic intervention.

### LEGEND TO THE FIGURES

FIG. 1. Relative expression of colon cancer markers in four colon tumor samples. RNA was purified from the tissue sample and assessed for the level of CGM2, cMyc, cyclin D1, CEA, PRL3, and GCCr. Values are plotted as the fold change from normal samples.

FIG. 2. Examples of Expression profile results. Oligonucleotide primers were designed to the novel event identified experimentally. Total RNA was isolated from matched samples for early stage colon tissue (normal and tumor) and late stage cancer. cDNA was prepared and end point RT-PCR analysis was performed to determine the level of expression for the event. cDNA's were normalized for GAPDH prior to the analysis. A) SEQ ID NO: 28; B) SEQ ID NO: 74.

Table 1. Markers for CRC used to evaluate tissue samples. The table consists of genes that are well known in colon cancer to have differential expression in tumor samples versus normal colon tissue. These genes were chosen to qualify the samples used to generate the DATAS™ libraries.

Table 2. Expression profiles for Expressed Sequence Tags in Colon Cancer. DATAS™, a differential expression analysis lead to the identification of Expressed Sequence Tags (EST's) that had the potential for serving as biomarkers in colon cancer. Primers were designed to detect the expression level of each EST by RT-PCR in two sets of samples derived from patients with either early or late stage colon cancer. Sequences are identified by the SEQ ID NO, the internal accession number, the GenBank accession number of the gene that is alternatively spliced, and the type of alternative splicing event: novel indicates the sequence suggested a novel exon present in the gene, extension indicates that a known exon from the gene contains additional sequence derived from the intron, leading to an extended exon; amplicon indicates that the sequence was derived by amplifying bioinformatically identified candidates and the detection of a novel splicing event. Expression was scored by the count of samples that were up or down regulated in cancer samples vs normal samples and expressed as a decimal (5.10) where the ones place (5) indicates the number of samples up-regulated, and the decimal (10) indicates the number of samples down regulated.

### DETAILED DESCRIPTION OF THE INVENTION

The present invention relates to novel target molecules suitable for monitoring, treating or developing cancer therapies, particularly colon cancer therapies.

The deregulation of RNA splicing in human disease is well documented and is supported by an exponentially increasing number of scientific publications. At least ten percent of germline mutations underlying human inherited disease affect RNA splicing, underlining the importance of this process in the development of disease. In addition, it has been estimated that 50% of all human genes undergo alternative splicing (Modrek et al., 2001; Kan et al., 2001). As examples of deregulation, there are isoforms that are specifically expressed in tumours (Obermair et al, 2001; Berggren et al., 2001; Milech et al., 2001; Lucas et al., 2001). In breast cancer, there is significant deregulation of splicing in the estrogen receptor alpha; normal breast tissue primarily expresses only a single variant, while breast tumours have an increased frequency of isoforms with multiple exon deletions (Poola and Speirs, 2001). Furthermore, deregulation of RNA splicing constitutes a mechanism by which response to chemotherapy may be altered. Alternative splicing profoundly affects normal biology, and when altered in a variety of systems can lead to disease.

DATAS™ (Different Analysis of Transcripts with Alternative Splicing) analyzes structural differences between expressed genes and provides systematic access to alterations in RNA splicing (disclosed in U.S. Pat. No. 6,251,590, the disclosure of which is incorporated by reference in its entirety). Having access to these spliced sequences, which are critical for the cellular homeostasis, represents a useful advance in functional genomics.

The DATAS™ Technology typically generates two libraries when comparing two samples, such as normal vs. tumor tissue. Each library specifically contains clones of sequences that are present and likely to be more highly expressed in one sample. For example, library A will contain sequences that are present in genes in the normal samples but absent (or expressed at lower levels) in the tumor samples. These sequences are identified as being removed or spliced out from the genes in the tumor samples. In contrast, library B will contain sequences that are present more abundantly and at higher concentrations in the tumor samples as compared to the normal samples. These represent exons/introns that are alternatively spliced into genes expressed predominantly in the tumor samples.

The present invention is based in part on the identification of exons that are isolated using DATAS™ and then determined to be differentially regulated or expressed in colon tumor samples. Specifically, 51 expressed sequences were identified through DATAS™ and confirmed to be differentially expressed between normal colon tissue and colon tumor tissue. These DATAS™ fragments (DF) are small sections of genes that are selected for inclusion or exclusion in one sample but not the other. These small sections are part of the expressed gene transcript, and can consist of sequences derived from several different regions of the gene, including, but not limited to, portions of single exons, several exons, sequence from introns, and sequences from exons and introns. This alternative usage of exons in different biological samples produces different gene products from the same gene through a process well known in the art as alternative RNA splicing. In the present application, 60 alternatively spliced isoforms have been identified from the DATAS™ fragment sequences, which produce alternate gene products that fit all the descriptions of target molecules as disclosed below.

Alternatively spliced mRNA's produced from the same gene contain different ribonucleotide sequence, and therefore translate into proteins with different amino acid sequences. Sequences that are alternatively spliced into or out of the gene products can be inserted or deleted in frame or out of frame from the original gene sequence. This leads to the translation of different proteins from each variant. Differences can include simple sequence deletions, or novel sequence information inserted into the gene product. Sequences inserted out of frame can lead to the production of an early stop codon and produce a truncated form of the protein. Many variations have been identified and produce protein variants that can be agonistic or antagonistic with the original biological activity of the protein.

The present invention thus identifies genes and proteins which are subject to differential regulation and alternative splicing(s) in colon cancer cells. The present invention thus provides target molecules suitable for diagnosis or therapy of colon cancers, which target molecules comprise all or a portion of genes or RNAs comprising the sequence of a DATAS™ fragment, or of genes or RNA from which the sequence of a DATAS™ fragment derives, as well as corresponding polypeptides or proteins, and variants thereof. These molecules also represent targets for diagnosis or therapy of other types of cancers, particularly those sharing the same type of deregulations as presently identified.

A first type of target molecule is a target nucleic acid molecule. Preferred target nucleic acid molecules comprise the sequence of a full gene or RNA molecule comprising the sequence of a DATAS™ fragment as disclosed in the present application, or a sequence complementary thereto. Indeed, since DATAS™ identifies genetic deregulations associated with colon tumor, the whole gene or RNA sequence from which said DATAS™ fragment derives can be used as a target of therapeutic intervention or diagnosis.

Additional target nucleic acid molecules comprise a fragment of a gene or RNA as disclosed above. Indeed, since DATAS™ identifies genes and RNAs that are altered in colon tumor cells, portions of such genes or RNAs, including portions that do not comprise the sequence of a DATAS™ fragment, can be used as a target for therapeutic intervention or diagnosis. Examples of such portions include: DATAS™ fragments, portions thereof, alternative exons or introns of said gene or RNA, junction sequences generated by exon splicing in said RNA, etc. Particular portions comprise a sequence encoding an extra-cellular domain of a polypeptide.

In this respect, a particular object of this invention resides in a nucleic acid molecule selected from the group consisting of:
(i) nucleic acids comprising a sequence contained in SEQ ID NOS. 1-44, 47, 52, 53, 56, 57, 62, 65, 70, 73, 74, 76, 79, 82, 85, 88, 91, and 96 or a sequence complementary thereto;
(ii) a nucleic acid having a sequence that is at least 70% identical to the sequence of (i) when aligned without allowing for gaps; and
(iii) fragments of (i) or (ii) having a size of at least 20 nucleotides in length.

Preferred fragments encode alternative exons or introns, junction sequences generated by exon splicing, or an extra-cellular domain of a polypeptide.

A second type of target molecule is represented by target polypeptides. In this regard, preferred target polypeptides comprise the sequence of a full-length protein comprising the amino acid sequence encoded by a DATAST fragment as disclosed in the present application or the corresponding whole gene or RNA.

Other target polypeptides of this invention are fragments of a protein as defined above. Such fragments may comprise or not the DATAS™ sequence, and may comprise newly generated amino acid sequence, resulting from a frame shift, the creation of new stop codon, etc.

A particular object of this invention resides more specifically in a polypeptide (or antigen) selected from the group consisting of:
(i) the polypeptide encoded by a nucleic acid sequence having at least 90% sequence identity in SEQ ID NOS. 1-44, 47, 52, 53, 56, 57, 62, 65, 70, 73, 74, 76, 79, 82, 85, 88, 91, and 96, or a sequence complementary thereto; and
(ii) the polypeptide comprising an amino acid sequence having at least 90% sequence identity in SEQ ID NOS. 48, 49, 54, 55, 58, 59, 66, 67, 71, 72, 75, 77, 78, 83, 84, 89, 90, 97, 98 and 99; and
(iii) an antigenic fragment of (i) or (ii).

These target molecules (including genes, fragments, proteins and their variants) can serve as diagnostic agents and as targets for the development of therapeutics. For example, these therapeutics may modulate biological processes associated with (colon) tumor formation, viability and/or growth. Agents may also be identified that are associated with the induction of apoptosis (cell death) in colon tumor cells. Other agents can also be developed, such as monoclonal antibodies, that bind to the protein or its variant and alter the biological processes important for cell growth. Alternatively, antibodies can deliver a toxin which can inhibit cell growth and lead to cell death.

Specifically, the invention provides sequences that are expressed in a variant protein and are colon tumor specific or colon specific. These sequences are portions of proteins identified to be in the plasma membrane of the cell, and the specific sequences of the invention are expressed on the extra-cellular region of the protein, so that the sequences may be useful in the preparation of (colon) tumor vaccines, including prophylatic and therapeutic vaccines.

Based thereon, it is anticipated that the disclosed genes that are associated with the differentially expressed sequences and the corresponding variant proteins represent suitable targets for cancer therapy, prevention or diagnosis, e.g. for the development of antibodies, small molecular inhibitors, inhibitory nucleic acids (e.g., anti-sense therapeutics, ribozymes, interfering RNAs, etc.), particularly for colon cancer. The potential therapies are described in greater detail below.

Inhibitory nucleic acids of this invention include oligonucleotides having sequences in the antisense orientation relative to the subject nucleic acids which appear to be unregulated in colon cancer. Suitable therapeutic inhibitory oligonucleotides typically vary in length from five to several hundred nucleotides, more typically about 20-70 nucleotides in length or shorter. These inhibitory oligonucleotides may be administered as naked nucleic acids or in protected forms, e.g., encapsulated in liposomes. The use of liposomal or other protected forms may be advantageous as it may enhance in vivo stability and thus facilitate delivery to target sites, e.g., colon tumor cells.

Also, the subject target genes may be used to design novel ribozymes that target the cleavage of the corresponding mRNAs in tumor cells. Similarly, these ribozymes may be administered in free (naked) form or by the use of delivery systems that enhance stability and/or targeting, e.g., liposomes.

Also, the subject target genes may be used to design novel siRNAs that can inhibit (e.g., reduce) expression of a target nucleic acid as disclosed in the present application. Similarly, these siRNAs may be administered in free (naked) form or by the use of delivery systems that enhance stability and/or targeting, e.g., liposomes. They may also be administered in the form of their precursors or encoding DNAs.

Also, the present invention embraces the administration of a ligand of a target molecule of this invention (e.g., a nucleic acid that hybridizes to the novel target nucleic acids identified infra or an antibody that specifically binds a target polypeptide as disclosed above), attached to therapeutic effector moieties, e.g., radiolabels (e.g., ⁹⁰Y, ¹³¹I), cytotoxins, cytotoxic enzymes, and the like in order to selectively target and kill cells that express these targets, e.g., colon tumor cells.

Also, the present invention embraces the treatment and/or diagnosis of cancer by targeting altered genes or the corresponding altered protein, particularly splice variants that are expressed in altered form in colon tumor cells, as described above. These methods provide for the selective detection of cells and/or eradication of cells that express such altered forms thereby minimizing adverse effects to normal cells.

Still further, the present invention encompasses other nucleic acid based therapies. For example, the invention encompasses the use of a DNA containing one of the novel cDNAs corresponding to novel antigen identified herein. It is anticipated that the antigens so encoded may be used as therapeutic or prophylactic anti-tumor vaccines. For example, a particular contemplated application of these antigens involves their administration with adjuvants that induce a cytotoxic T lymphocyte response.

Administration of the subject novel antigens in combination with an adjuvant may result in a humoral immune response against such antigens, thereby delaying or preventing the development of cancer.

These embodiments of the invention comprise, for instance, administration of one or more of the target polypeptides of this invention, or antigenic fragments thereof, typically in combination with an adjuvant. Such compositions shall be administered in an amount sufficient to be therapeutically or prophylactically effective, e.g. on the order of 50 to 20,000 mg/kg body weight, 100 to 5000 mg/kg body weight. Suitable adjuvants for use in the present invention include PROVAX™, which comprises a microfluidized adjuvant containing Squalene, Tween and Pluronic, ISCOM'S®, DETOX®, SAF, Freund's adjuvant, Alum® and Saponin®, among others.

Yet another embodiment of the invention comprises the preparation of monoclonal antibodies against a target polypeptide s defined above. Such monoclonal antibodies may be produced by conventional methods and include fragments or derivatives thereof, including, without limitation, human monoclonal antibodies, humanized monoclonal antibodies, chimeric monoclonal antibodies, single chain antibodies, e.g., scFv's and antigen-binding antibody fragments such as Fab and Fab′ fragments. Methods for the preparation of monoclonal antibodies are known in the art. In general, the preparation of monoclonal antibodies comprises immunization of an appropriate (non-homologous) host with the subject colon cancer antigens, isolation of immune cells therefrom, use of such immune cells to isolate monoclonal antibodies and screening for monoclonal antibodies that specifically bind to either of such antigens. Antibody fragments may be prepared by known methods, e.g., enzymatic cleavage of monoclonal antibodies.

These monoclonal antibodies and fragments are useful for passive anti-tumor immunotherapy, or may be attached to therapeutic effector moieties, e.g., radiolabels, cytotoxins, therapeutic enzymes, agents that induce apoptosis, and the like in order to provide for targeted cytotoxicity, i.e., killing of human colon tumor cells. Given the fact that the subject genes are apparently not significantly expressed by many normal tissues this should not result in significant adverse side effects (toxicity to non-target tissues).

In one embodiment of the present invention, such antibodies or fragments are administered in labeled or unlabeled form, alone or in conjunction with other therapeutics, e.g., chemotherapeutics such as cisplatin, methotrexate, adriamycin, and the like suitable for cancer therapy. The administered composition typically includes a pharmaceutically acceptable carrier, and optionally adjuvants, stabilizers, etc., used in antibody compositions for therapeutic use.

Preferably, the subject monoclonal antibodies binds the target antigens with high affinity, e.g., possess a binding affinity (Kd) on the order of 10⁻⁶ to 10⁻¹² M.

The present invention also embraces diagnostic applications that provide for detection of the expression of colon specific splice variants disclosed herein. This comprises detecting the expression of one or more of these genes at the RNA level and/or at the protein level.

In this respect, a particular object of this invention resides in methods of detecting and/or staging cancer, particularly colon cancer, comprising (i) obtaining a human cell sample, particularly a human colon cell sample; and (ii) determining whether such cell sample expresses a target molecule, wherein said target molecule comprises the sequence of a gene or RNA comprising a nucleic acid sequence selected from the group consisting of SEQ ID NOS. 1-44, 47, 52, 53, 56, 57, 62, 65, 70, 73, 74, 76, 79, 82, 85, 88, 91, and 96; a sequence complementary thereto, or of a fragment of said gene or RNA having a size of at least 20 nucleotides in length, or an amino acid sequence encoded by such a nucleic acid. Determination of expression may comprise quantitative and/or qualitative evaluations, e.g., absolute and/or relative measure of such expression levels. Typically, the expression level of said target molecule in said cell sample is compared to a reference expression level, wherein a deviation from said reference expression level is indicative of the presence and/or stage of said cancer in said subject. The reference expression level may be an expression level as determined in a control sample (e.g., from a healthy tissue or subject) or a median expression level from healthy subjects. A “deviation” from said reference expression level designates any significant change, such as an increase or decrease by at least 10%, 20%, or 30%, preferably by at least 40% or 50%, or even more.

For nucleic acids, expression of the subject genes can be detected by known nucleic acid detection methods, e.g., Northern blot hybridization, strand displacement amplification (SDA), catalytic hybridization amplification (CHA), and other known nucleic acid detection methods. Preferably, a cDNA library will be made from colon cells obtained from a subject to be tested for colon cancer by PCR using primers corresponding to the novel isoforms disclosed in this application.

The presence or absence of a cancer can be determined based on whether PCR products are obtained, and the level of expression. The levels of expression of such PCR product may be quantified in order to determine the prognosis of a particular cancer patient, particularly a colon cancer patient (as the levels of expression of the PCR product often will increase or decrease significantly as the disease progresses.) This may provide a method for monitoring the status of a cancer patient.

Alternatively, the status of a subject to be tested for cancer may be evaluated by testing biological fluids (e.g., blood, urine, lymph), bodily excretions (e.g. fecal matter), exfoliated colonocytes, and the like with an antibody or antibodies or fragment that specifically binds to the novel tumor antigens disclosed herein.

Methods for using antibodies to detect antigen expression are well known and include ELISA, competitive binding assays, and the like. In general, such assays use an antibody or antibody fragment that specifically binds the target antigen directly or indirectly bound to a label that provides for detection, e.g. indicator enzymes, a radiolabels, fluorophores, or paramagnetic particles.

Patients which test positive for the enhanced presence of the antigen on cancer cells will be diagnosed as having or being at increased risk of developing cancer. Additionally, the levels of antigen expression may be useful in determining patient status, i.e., how far disease has advanced (stage of cancer).

As noted, the present invention provides novel splice variants that encode antigens that correlate to human cancer. The present invention also embraces variants thereof. As used herein “variants” means sequences that are at least about 75% identical thereto, more preferably at least about 85% identical, and most preferably at least 90% identical and still more preferably at least about 95-99% identified when these DNA sequences are compared to a nucleic acid sequence encoding the subject DNAs or a fragment thereof having a size of at least about 50 nucleotides. This includes allelic and splice variants of the subject genes. The present invention also encompasses nucleic acid sequences that hybridize to the subject splice variants under high, moderate or low stringency conditions e.g., as described infra.

Also, the present invention provides for primer pairs that result in the amplification of DNAs encoding the subject novel genes or a portion thereof in an mRNA library obtained from a desired cell source, typically human colon cell or tissue sample. Typically, such primers will be on the order of 12 to 50 nucleotides in length, and will be constructed such that they provide for amplification of the entire or most of the target gene.

Also, the invention embraces the antigens encoded by the subject DNAs or fragments thereof that bind to or elicits antibodies specific to the full-length antigens. Typically, such fragments will be at least 10 amino acids in length, more typically at least 25 amino acids in length.

As noted, the subject DNA fragments are expressed in a majority of colon tumor samples tested. The invention further contemplates the identification of other cancers that express such genes and the use thereof to detect and treat such cancers. For example, the subject DNA fragments or variants thereof may be expressed on other cancers, e.g., breast, ovary, pancreas, lung or colon cancers. Essentially, the present invention embraces the detection of any cancer wherein the expression of the subject novel genes or variants thereof correlate to a cancer or an increased likelihood of cancer. To facilitate under-study of the invention, the following definitions are provided.

“Isolated tumor antigen or tumor protein” refers to any protein that is not in its normal cellular milieu. This includes by way of example compositions comprising recombinant proteins encoded by the genes disclosed infra, pharmaceutical compositions comprising such purified proteins, diagnostic compositions comprising such purified proteins, and isolated protein compositions comprising such proteins. In preferred embodiments, an isolated colon tumor protein according to the invention will comprise a substantially pure protein, in that it is substantially free of other proteins, preferably that is at least 90% pure, that comprises the amino acid sequence contained herein or natural homologues or mutants having essentially the same sequence. A naturally occurring mutant might be found, for instance, in tumor cells expressing a gene encoding a mutated protein according to the invention.

“Native tumor antigen or tumor protein” refers to a protein that is a non-human primate homologue of the protein having the amino acid sequence contained infra.

“Isolated colon tumor gene or nucleic acid sequence” refers to a nucleic acid molecule that encodes a tumor antigen according to the invention which is not in its normal human cellular milieu, e.g., is not comprised in the human or non-human primate chromosomal DNA. This includes by way of example vectors that comprise a gene according to the invention, a probe that comprises a gene according to the invention, and a nucleic acid sequence directly or indirectly attached to a detectable moiety, e.g. a fluorescent or radioactive label, or a DNA fusion that comprises a nucleic acid molecule encoding a gene according to the invention fused at its 5′ or 3′ end to a different DNA, e.g. a promoter or a DNA encoding a detectable marker or effector moiety. Also included are natural homologues or mutants having substantially the same sequence. Naturally occurring homologies that are degenerate would encode the same protein including nucleotide differences that do not change the corresponding amino acid sequence. Naturally occurring mutants might be found in tumor cells, wherein such nucleotide differences may result in a mutant tumor antigen. Naturally occurring homologues containing conservative substitutions are also encompassed.

“Variant of colon tumor antigen or tumor protein” refers to a protein possessing an amino acid sequence that possess at least 90% sequence identity, more preferably at least 91% sequence identity, even more preferably at least 92% sequence identity, still more preferably at least 93% sequence identity, still more preferably at least 94% sequence identity, even more preferably at least 95% sequence identity, still more preferably at least 96% sequence identity, even more preferably at least 97% sequence identity, still more preferably at least 98% sequence identity, and most preferably at least 99% sequence identity, to the corresponding native tumor antigen wherein sequence identity is as defined infra. Preferably, this variant will possess at least one biological property in common with the native protein.

“Variant of colon tumor gene or nucleic acid molecule or sequence” refers to a nucleic acid sequence that possesses at least 90% sequence identity, more preferably at least 91%, more preferably at least 92%, even more preferably at least 93%, still more preferably at least 94%, even more preferably at least 95%, still more preferably at least 96%, even more preferably at least 97%, even more preferably at least 98% sequence identity, and most preferably at least 99% sequence identity, to the corresponding native human nucleic acid sequence, wherein “sequence identity” is as defined infra.

“Fragment of colon antigen encoding nucleic acid molecule or sequence” refers to a nucleic acid sequence corresponding to a portion of the native human gene wherein said portion is at least about 50 nucleotides in length, or 100, more preferably at least 150 nucleotides in length.

“Antigenic fragments of colon tumor antigen” refer to polypeptides corresponding to a fragment of a colon protein or a variant or homologue thereof that when used itself or attached to an immunogenic carrier elicits antibodies that specifically bind the protein. Typically such antigenic fragments will be at least 8-15 amino acids in length, and may be much longer.

Sequence identity or percent identity is intended to mean the percentage of the same residues shared between two sequences, referenced to human protein A or protein B or gene A or gene B, when the two sequences are aligned using the Clustal method [Higgins et al, Cabios 8:189-191 (1992)] of multiple sequence alignment in the Lasergene biocomputing software (DNASTAR, INC, Madison, Wis.), or alignment programs available from the Genetics Computer Group (GCG Wisconsin package, Accelrys, San Diego, Calif.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment—5; diagonals saved in pairwise alignment=5. The residue weight table used for the alignment program is PAM25O [Dayhoff et al., in Atlas of Protein Sequence and Structure, Dayhoff, Ed., NDRF, Washington, Vol. 5, suppl. 3, p. 345, (1978)].

Percent conservation is calculated from the above alignment by adding the percentage of identical residues to the percentage of positions at which the two residues represent a conservative substitution (defined as having a log odds value of greater than or equal to 0.3 in the PAM250 residue weight table). Conservation is referenced to human Gene A or gene B when determining percent conservation with non-human Gene A or gene B, e.g. gene A or gene B, when determining percent conservation. Conservative amino acid changes satisfying this requirement include: R-K; E-D, Y-F, L-M; V-I, Q-H.

### Polypeptide Fragments

The invention provides polypeptide fragments of the disclosed proteins. Polypeptide fragments of the invention can comprise at least 8, more preferably at least 25, still more preferably at least 50 amino acid residues of the protein or an analogue thereof. More particularly such fragment will comprise at least 75, 100, 125, 150, 175, 200, 225, 250, 275 residues of the polypeptide encoded by the corresponding gene. Even more preferably, the protein fragment will comprise the majority of the native protein, e.g. about 100 contiguous residues of the native protein.

### Biologically Active Variants

The invention also encompasses mutants of the novel colon proteins disclosed infra which comprise an amino acid sequence that is at least 80%, more preferably 90%, still more preferably 95-99% similar to the native protein.

Guidance in determining which amino acid residues can be substituted, inserted, or deleted without abolishing biological or immunological activity can be found using computer programs well known in the art, such as DNASTAR or software from the Genetics Computer Group (GCG). Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

A subset of mutants, called muteins, is a group of polypeptides in which neutral amino acids, such as serines, are substituted for cysteine residues which do not participate in disulfide bonds. These mutants may be stable over a broader temperature range than native secreted proteins. See Mark et al., U.S. Pat. No. 4,959,314.

It is reasonable to expect that an isolated replacement of a leucine with an isoleucine or valine, an aspartate with a glutamnate, a threonine with a serine, or a similar replacement of an amino acid with a structurally related amino acid will not have a major effect on the biological properties of the resulting secreted protein or polypeptide variant.

Protein variants include glycosylated forms, aggregative conjugates with other molecules, and covalent conjugates with unrelated chemical moieties. Also, protein variants also include allelic variants, species variants, and muteins. Truncations or deletions of regions which do not affect the differential expression of the gene are also variants. Covalent variants can be prepared by linking functionalities to groups which are found in the amino acid chain or at the N- or C-terminal residue, as is known in the art.

It will be recognized in the art that some amino acid sequence of the colon proteins of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Ostade et al., Nature 361:266-268 (1993) describes certain mutations resulting in selective binding of TNF-alpha to only one of the two known types of TNF receptors. Thus, the polypeptides of the present invention may include one or more amino acid substitutions, deletions or additions, either from natural mutations or human manipulation.

The invention further includes variations of the colon proteins disclosed infra which show comparable expression patterns or which include antigenic regions. Such mutants include deletions, insertions, inversions, repeats, and site substitutions. Guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic. (Pinckard et al., Clin. Exp. Immunol. 2:331-340 (1967); Robbins et al., Diabetes 36:838-845 (1987); Cleland et al., Crit. Rev. Therapeutic Drug Carrier Systems 10:307-377 (1993)).

Amino acids in the polypeptides of the present invention that are essential for function can be identified by methods known in the art, such as site-directed mutagenesis or alanine-scanning mutagenesis (Cunningham and Wells, Science 244: 1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255: 306-312 (1992)).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given polypeptide will not be more than 50, 40, 30, 25, 20, 15, 10, 5 or 3.

### Fusion Proteins

Fusion proteins comprising proteins or polypeptide fragments of the subject colon tumor antigen can also be constructed. Fusion proteins are useful for generating antibodies against amino acid sequences and for use in various assay systems. For example, fusion proteins can be used to identify proteins which interact with a protein of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of a protein according to the invention or a fragment thereof can be used to target other protein domains to cellular locations in which the domains are not normally found, such as bound to a cellular membrane or secreted extracellularly.

A fusion protein comprises two protein segments fused together by means of a peptide bond. As noted, these fragments may range in size from about 8 amino acids up to the full length of the protein.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP 16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding sequence encoding a possible antigen according to the invention or a fragment thereof in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Proteins, fusion proteins, or polypeptides of the invention can be produced by recombinant DNA methods. For production of recombinant proteins, fusion proteins, or polypeptides, a sequence encoding the protein can be expressed in prokaryotic or eukaryotic host cells using expression systems known in the art. These expression systems include bacterial, yeast, insect, and mammalian cells.

The resulting expressed protein can then be purified from the culture medium or from extracts of the cultured cells using purification procedures known in the art. For example, for proteins fully secreted into the culture medium, cell-free medium can be diluted with sodium acetate and contacted with a cation exchange resin, followed by hydrophobic interaction chromatography. Using this method, the desired protein or polypeptide is typically greater than 95% pure. Further purification can be undertaken, using, for example, any of the techniques listed above.

It may be necessary to modify a protein produced in yeast or bacteria, for example by phosphorylation or glycosylation of the appropriate sites, in order to obtain a functional protein. Such covalent attachments can be made using known chemical or enzymatic methods.

A protein or polypeptide of the invention can also be expressed in cultured host cells in a form which will facilitate purification. For example, a protein or polypeptide can be expressed as a fusion protein comprising, for example, maltose binding protein, glutathione-S-transferase, or thioredoxin, and purified using a commercially available kit. Kits for expression and purification of such fusion proteins are available from companies such as New England BioLabs, Pharmacia, and Invitrogen. Proteins, fusion proteins, or polypeptides can also be tagged with an epitope, such as a “Flag” epitope (Kodak), and purified using an antibody which specifically binds to that epitope.

The coding sequence of the protein variants identified through the sequences disclosed herein can also be used to construct transgenic animals, such as mice, rats, guinea pigs, cows, goats, pigs, or sheep. Female transgenic animals can then produce proteins, polypeptides, or fusion proteins of the invention in their milk. Methods for constructing such animals are known and widely used in the art.

Alternatively, synthetic chemical methods, such as solid phase peptide synthesis, can be used to synthesize a secreted protein or polypeptide. General means for the production of peptides, analogs or derivatives are outlined in Chemistry and Biochemistry of Amino Acids, Peptides, and Proteins—A Survey of Recent Developments, B. Weinstein, ed. (1983). Substitution of D-amino acids for the normal L-stereoisomer can be carried out to increase the half-life of the molecule.

Typically, homologous polynucleotide sequences can be confirmed by hybridization under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each, homologous sequences can be identified which contain at most about 25-30% basepair mismatches. More preferably, homologous nucleic acid strands contain 15-25% basepair mismatches, even more preferably 5-15% basepair mismatches.

The invention also provides polynucleotide probes which can be used to detect complementary nucleotide sequences, for example, in hybridization protocols such as Northern or Southern blotting or in situ hybridizations. Polynucleotide probes of the invention comprise at least 12, 13, 14, 15, 16, 17, 18, 19, 20, 30, or 40 or more contiguous nucleotides of the nucleic acid sequences provided herein. Polynucleotide probes of the invention can comprise a detectable label, such as a radioisotopic, fluorescent, enzymatic, or chemiluminescent label.

Isolated genes corresponding to the cDNA sequences disclosed herein are also provided. Standard molecular biology methods can be used to isolate the corresponding genes using the cDNA sequences provided herein. These methods include preparation of probes or primers from the nucleotide sequence disclosed herein for use in identifying or amplifying the genes from mammalian, including human, genomic libraries or other sources of human genomic DNA.

Polynucleotide molecules of the invention can also be used as primers to obtain additional copies of the polynucleotides, using polynucleotide amplification methods. Polynucleotide molecules can be propagated in vectors and cell lines using techniques well known in the art. Polynucleotide molecules can be on linear or circular molecules. They can be on autonomously replicating molecules or on molecules without replication sequences. They can be regulated by their own or by other regulatory sequences, as is known in the art.

### Polynucleotide Constructs

Polynucleotide molecules comprising the coding sequences of the gene variants identified through the sequences disclosed herein can be used in a polynucleotide construct, such as a DNA or RNA construct. Polynucleotide molecules of the invention can be used, for example, in an expression construct to express all or a portion of a protein, variant, fusion protein, or single-chain antibody in a host cell. An expression construct comprises a promoter which is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment which encodes all or a portion of the desired protein. The polynucleotide segment is located downstream from the promoter. Transcription of the polynucleotide segment initiates at the promoter. The expression construct can be linear or circular and can contain sequences, if desired, for autonomous replication.

Also included are polynucleotide molecules comprising the promoter and UTR sequences of the subject novel genes, operably linked to the associated protein coding sequence and/or other sequences encoding a detectable or selectable marker. Such promoter and/or UTR-based constructs are useful for studying the transcriptional and translational regulation of protein expression, and for identifying activating and/or inhibitory regulatory proteins.

### Host Cells

An expression construct can be introduced into a host cell. The host cell comprising the expression construct can be any suitable prokaryotic or eukaryotic cell. Expression systems in bacteria include those described in Chang et al., Nature 275:615 (1978); Goeddel et al., Nature 281: 544 (1979); Goeddel et al., Nucleic Acids Res. 8:4057 (1980); EP 36,776; U.S. Pat. No. 4,551,433; deBoer et al., Proc. Natl. Acad. Sci. USA 80: 21-25 (1983); and Siebenlist et al., Cell 20: 269 (1980).

Expression systems in yeast include those described in Hinnnen et al., Proc. Natl. Acad. Sci. USA 75: 1929 (1978); Ito et al., J Bacteriol 153: 163 (1983); Kurtz et al., Mol. Cell. Biol. 6: 142 (1986); Kunze et al., J Basic Microbiol. 25: 141 (1985); Gleeson et al., J. Gen. Microbiol. 132: 3459 (1986), Roggenkamp et al., Mol. Gen. Genet. 202: 302 (1986)); Das et al., J Bacteriol. 158: 1165 (1984); De Louvencourt et al., J Bacteriol. 154:737 (1983), Van den Berg et al., Bio/Technology 8: 135 (1990); Kunze et al., J. Basic Microbiol. 25: 141 (1985); Cregg et al., Mol. Cell. Biol. 5: 3376 (1985); U.S. Pat. No. 4,837,148; U.S. Pat. No. 4,929,555; Beach and Nurse, Nature 300: 706 (1981); Davidow et al., Curr. Genet. 10: 380 (1985); Gaillardin et al., Curr. Genet. 10: 49 (1985); Ballance et al., Biochem. Biophys. Res. Commun. 112: 284-289 (1983); Tilburn et al., Gene 26: 205-22 (1983); Yelton et al., Proc. Natl. Acad, Sci. USA 81: 1470-1474 (1984); Kelly and Hynes, EMBO J. 4: 475479 (1985); EP 244,234; and WO 91/00357.

Expression of heterologous genes in insects can be accomplished as described in U.S. Pat. No. 4,745,051; Friesen et al. (1986) “The Regulation of Baculovirus Gene Expression” in: THE MOLECULAR BIOLOGY OF BACULOVIRUSES (W. Doerfler, ed.); EP 127,839; EP 155,476; Vlak et al., J. Gen. Virol. 69: 765-776 (1988); Miller et al., Ann. Rev. Microbiol. 42: 177 (1988); Carbonell et al., Gene 73: 409 (1988); Maeda et al., Nature 315: 592-594 (1985); Lebacq-Verheyden et al., Mol. Cell. Biol. 8: 3129 (1988); Smith et al., Proc. Natl. Acad. Sci. USA 82: 8404 (1985); Miyajima et al., Gene 58: 273 (1987); and Martin et al., DNA 7:99 (1988). Numerous baculoviral strains and variants and corresponding permissive insect host cells from hosts are described in Luckow et al., Bio/Technology (1988) δ: 47-55, Miller et al., in GENETIC ENGINEERING (Setlow, J. K. et al. eds.), Vol. 8, pp. 277-279 (Plenum Publishing, 1986); and Maeda et al., Nature, 315: 592-594 (1985).

Mammalian expression can be accomplished as described in Dijkema et al., EMBO J. 4: 761 (1985); Gorman et al., Proc. Natl. Acad. Sci. USA 79: 6777 (1982b); Boshart et al., Cell 41: 521 (1985); and U.S. Pat. No. 4,399,216. Other features of mammalian expression can be facilitated as described in Ham and Wallace, Meth Enz. 58: 44 (1979);

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

The invention can also include hybrid and modified forms thereof including fusion proteins, fragments and hybrid and modified forms in which certain amino acids have been deleted or replaced, modifications such as where one or more amino acids have been changed to a modified amino acid or unusual amino acid.

Also included within the meaning of substantially homologous is any human or non-human primate protein which may be isolated by virtue of cross-reactivity with antibodies to proteins encoded by a gene described herein or whose encoding nucleotide sequences including genomic DNA, mRNA or cDNA may be isolated through hybridization with the complementary sequence of genomic or subgenomic nucleotide sequences or cDNA of a gene herein or fragments thereof. It will also be appreciated by one skilled in the art that degenerate DNA sequences can encode a tumor protein according to the invention and these are also intended to be included within the present invention as are allelic variants of the subject genes.

Preferred is a colon protein according to the invention prepared by recombinant DNA technology. By “pure form” or “purified form” or “substantially purified form” it is meant that a protein composition is substantially free of other proteins which are not the desired protein.

The present invention also includes therapeutic or pharmaceutical compositions comprising a protein according to the invention in an effective amount for treating patients with disease, and a method comprising administering a therapeutically effective amount of the protein. These compositions and methods are useful for treating cancers associated with the subject proteins, e.g. colon cancer. One skilled in the art can readily use a variety of assays known in the art to determine whether the protein would be useful in promoting survival or functioning in a particular cell type.

Anti-Colon Antigen Antibodies

As noted, the invention includes the preparation and use of anti-colon antigen antibodies and fragments for use as diagnostics and therapeutics. These antibodies may be polyclonal or monoclonal. Polyclonal antibodies can be prepared by immunizing rabbits or other animals by injecting antigen followed by subsequent boosts at appropriate intervals. The animals are bled and sera assayed against purified protein usually by ELISA or by bioassay based upon the ability to block the action of the corresponding gene. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. [Milstein and Kohler, Nature 256:495-497 (1975); Gulfre and Milstein, Methods in Enzymology: Immunochemical Techniques 73:1-46, Langone and Banatis eds., Academic Press, (1981) which are incorporated by reference]. The hybridoma cells so formed are then cloned by limiting dilution methods and supernates assayed for antibody production by ELISA, RIA or bioassay.

The unique ability of antibodies to recognize and specifically bind to target proteins provides an approach for treating an overexpression of the protein. Thus, another aspect of the present invention provides for a method for preventing or treating diseases involving overexpression of the protein by treatment of a patient with specific antibodies to the protein.

Specific antibodies, either polyclonal or monoclonal, to the protein can be produced by any suitable method known in the art as discussed above. For example, by recombinant methods, preferably in eukaryotic cells murine or human monoclonal antibodies can be produced by hybridoma technology or, alternatively, the protein, or an immunologically active fragment thereof, or an anti-idiotypic antibody, or fragment thereof can be administered to an animal to elicit the production of antibodies capable of recognizing and binding to the protein. Such antibodies can be from any class of antibodies including, but not limited to IgG, IgA, 1 gM, IgD, and IgE or in the case of avian species, IgY and from any subclass of antibodies.

The availability of isolated protein allows for the identification of small molecules and low molecular weight compounds that inhibit the binding of protein to binding partners, through routine application of high-throughput screening methods (HTS). HTS methods generally refer to technologies that permit the rapid assaying of lead compounds for therapeutic potential. HTS techniques employ robotic handling of test materials, detection of positive signals, and interpretation of data. Lead compounds may be identified via the incorporation of radioactivity or through optical assays that rely on absorbance, fluorescence or luminescence as read-outs. [Gonzalez, J. E. et al, Curr. Opin. Biotech. 9:624-63 1 (1998)].

Model systems are available that can be adapted for use in high throughput screening for compounds that inhibit the interaction of protein with its ligand, for example by competing with protein for ligand binding. Sarubbi et al., Anal. Biochem. 237:70-75 (1996) describe cell-free, non-isotopic assays for discovering molecules that compete with natural ligands for binding to the active site of IL-1 receptor. Martens, C. et al., Anal. Biochem. 273:20-31 (1999) describe a generic particle-based nonradioactive method in which a labeled ligand binds to its receptor immobilized on a particle; label on the particle decreases in the presence of a molecule that competes with the labeled ligand for receptor binding.

### (i) Starting Materials and Methods

Immunoglobulins (Ig) and certain variants thereof are known and many have been prepared in recombinant cell culture. For example, see U.S. Pat. No. 4,745,055; EP 256,654; EP 120,694; EP 125,023; EP 255,694; EP 266,663; WO 30 88/03559; Faulkneret al., Nature, 298: 286 (1982); Morrison, J. Immun., 123: 793 (1979); Koehler et al., Proc. Natl. Acad. Sci. USA, 77: 2197 (1980); Raso et al., Cancer Res., 41: 2073 (1981); Morrison et al., Ann. Rev. Immunol., 2: 239 (1984); Morrison, Science, 229: 1202 (1985); and Morrison et al., Proc. Natl. Acad. Sci. USA, 81: 6851 (1984). Reassorted immunoglobulin chains are also known. See, for example, U.S. Pat. No. 4,444,878; WO 88/03565; and EP 68,763 and references cited therein. The immunoglobulin moiety in the chimeras of the present invention may be obtained from IgG-1, IgG-2, IgG-3, or IgG-4 subtypes, IgA, IgE, IgD, or IgM, but preferably from IgG-1 or IgG-3.

### (ii) Polyclonal Antibodies

Polyclonal antibodies to the subject colon antigens are generally raised in animals by multiple subcutaneous (sc) or intraperitoneal (ip) injections of the antigen and an adjuvant. It may be useful to conjugate the antigen or a fragment containing the target amino acid sequence to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde or succinic anhydride.

Animals are immunized against the polypeptide or fragment, immunogenic conjugates, or derivatives by combining about 1 mg or 1 μg of the peptide or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer to the antigen or a fragment thereof. Animals are boosted until the titer plateaus. Preferably, the animal is boosted with the conjugate of the same polypeptide or fragment thereof, but conjugated to a different protein and/or through a different cross-linking reagent. Conjugates also can be made in recombinant cell culture as protein fusions. Also, aggregating agents such as alum are suitably used to enhance the immune response.

(iii) Monoclonal Antibodies

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, monoclonal antibodies using for practicing this invention may be made using the hybridoma method first described by Kohler and Milstein, Nature, 256: 495 (1975), or may be made by recombinant DNA methods (Cabilly et al., supra).

In the hybridoma method, a mouse or other appropriate host animal, such as a hamster, is immunized as hereinabove described to elicit lymphocytes that produce or are capable of producing antibodies that will specifically bind to the antigen or fragment thereof used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 [Academic Press, 1986]).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Culture medium in which hybridoma cells are growing is assayed for production of monoclonal antibodies directed against the colon antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson and Pollard, Anal. Biochem., 107: 220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxyapatite chromatography, gel electrophoresis, dialysis, or affinity chromatography.

DNA encoding the monoclonal antibodies of the invention is readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5: 256-262 (1993) and Pluckthun, Immunol. Revs., 130: 151-188 (1992). A preferred expression system is the NEOSPLA™ expression system of IDEC above-referenced.

The DNA also may be modified, for example, by substituting the coding sequence for human heavy- and light-chain constant domains in place of the homologous murine sequences (Morrison, et al., Proc. Natl. Acad. Sci. USA, 81: 6851 [1984]), or by covalently joining to the immunoglobulin coding sequence all or part of the coding sequence for a non-immunoglobulin polypeptide. In that manner, “chimeric” or “hybrid” antibodies are prepared that have the binding specificity of an anti-colon antigen monoclonal antibody herein.

Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for colon antigen according to the invention and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### (iv) Humanized Antibodies

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature 321, 522-525 [1986]; Riechmann et al., Nature 332, 323-327 [1988]; Verhoeyen et al., Science 239, 1534-1536 [1988]), by substituting rodent CDRs or CDR sequences for the corresponding sequences of a human antibody. Accordingly, such “humanized” antibodies are chimeric antibodies (Cabilly et al., supra), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151: 2296 [1993]; Chothia and Lesk, J. Mol. Biol., 196: 901 [1987]). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89: 4285 [1992]; Presta et al., J. Immunol., 151: 2623 [1993]).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding.

### (v) Human Antibodies

Human monoclonal antibodies can be made by the hybridoma method. Human myeloma and mouse-human heteromyeloma cell lines for the production of human monoclonal antibodies have been described, for example, by Kozbor, J. Immunol. 133, 3001 (1984); Brodeur, et al., Monoclonal Antibody Production Techniques and Applications, pp. 51-63 (Marcel Dekker, Inc., New York, 1987); and Boerner et al., J. Immunol., 147: 86-95 (1991).

It is now possible to produce transgenic animals (e.g., mice) that are capable, upon immunization, of producing a fall repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90: 2551 (1993); Jakobovits et al., Nature, 362: 255-258 (1993); Bruggermann et al., Year in Immuno., 7: 33 (1993).

Alternatively, the phage display technology (McCafferty et al., Nature, 348: 552-553 [1990]) can be used to produce human antibodies and antibody fragments in vitro, from immunoglobulin variable (V) domain gene repertoires from non-immunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson and Chiswell, Curr. Op. Struct. Biol., 3: 564-571 (1993). Several sources of V-gene segments can be used for phage display. Clackson et al., Nature, 352: 624-628 (1991) isolated a diverse array of anti-oxazolone antibodies from a small random combinatorial library of V genes derived from the spleens of immunized mice. A repertoire of V genes from non-immunized human donors can be constructed and antibodies to a diverse array of antigens (including self-antigens) can be isolated essentially following the techniques described by Marks et al., J. Mol. Biol., 222: 581-597 (1991), or Griffith et al., EMBO J., 12: 725-734 (1993).

In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., Bio/Technology, 10: 779-783 [1992]). In this method, the affinity of “primary” human antibodies obtained by phage display can be improved by sequentially replacing the heavy and light chain V region genes with repertoires of naturally occurring variants (repertoires) of V domain genes obtained from non-immunized donors. This technique allows the production of antibodies and antibody fragments with affinities in the nM range. A strategy for making very large phage antibody repertoires has been described by Waterhouse et al., Nucl. Acids Res., 21: 2265-2266 (1993).

Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e., the epitope governs (imprints) the choice of partner. When the process is repeated in order to replace the remaining rodent V domain, a human antibody is obtained (see PCT WO 93/06213, published Apr. 1, 1993). Unlike traditional humanization of rodent antibodies by CDR grafting, this technique provides completely human antibodies, which have no framework or CDR residues of rodent origin.

### (vi) Bispecific Antibodies

Bispecific antibodies are monoclonal, preferably human or humanized, antibodies that have binding specificities for at least two different antigens. In the present case, one of the binding specificities will be to a colon antigen according to the invention. Methods for making bispecific antibodies are known in the art.

Traditionally, the recombinant production of bispecific antibodies is based on the co-expression of two immunoglobulin heavy chain-light chain pairs, where the two heavy chains have different specificities (Milstein and Cuello, Nature, 305: 537-539 [1983]). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in WO 93/08829 published May 13, 1993, and in Traunecker et al., EMBO J., 10: 3655-3659 (1991).

According to a different and more preferred approach, antibody-variable domains with the desired binding specificities (antibody-antigencombining sites) are fused to immunoglobulin constant-domain sequences. The fusion preferably is with an immunoglobulin heavy-chain constant domain, comprising at least part of the hinge, CH2, and CH3 regions. It is preferred to have the first heavy-chain constant region (CH1), containing the site necessary for light-chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the production of at least two polypeptide chains in equal ratios results in high yields or when the ratios are of no particular significance. In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation.

For further details of generating bispecific antibodies, see, for example, Suresh et al., Methods in Enzymology, 121: 210 (1986).

(vii) Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/00373; and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

The polynucleotides and polypeptides of the present invention may be utilized in gene delivery vehicles. The gene delivery vehicle may be of viral or non-viral origin (see generally, Jolly, Cancer Gene Therapy 1:51-64 (1994); Kimura, Human Gene Therapy 5:845-852 (1994); Connelly, Human Gene Therapy 1:185-193 (1995); and Kaplitt, Nature Genetics 6:148-153 (1994)). Gene therapy vehicles for delivery of constructs including a coding sequence of a therapeutic according to the invention can be administered either locally or systemically. These constructs can utilize viral or non-viral vector approaches. Expression of such coding sequences can be induced using endogenous mammalian or heterologous promoters. Expression of the coding sequence can be either constitutive or regulated. Preferred vehicles for gene therapy include retroviral and adeno-viral vectors.

Representative examples of adenoviral vectors include those described by Berkner, Biotechniques 6:616-627 (Biotechniques); Rosenfeld et al., Science 252:431-434 (1991); WO 93/19191; Kolls et al., P.N.A.S. 215-219 (1994); Kass-Bisleret al., P.N.A.S. 90: 11498-11502 (1993); Guzman et al., Circulation 88: 2838-2848 (1993); Guzman et al., Cir. Res. 73: 1202-1207 (1993); Zabner et al., Cell 75: 207-216 (1993); Li et al., Hum. Gene Ther. 4: 403-409 (1993); Cailaud et al., Eur. J. Neurosci. 5: 1287-1291 (1993); Vincent et al., Nat. Genet. 5: 130-134 (1993); Jaffe et al., Nat. Genet. 1: 372-378 (1992); and Levrero et al., Gene 101: 195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to kill adenovirus as described in Curiel, Hum. Gene Ther. 3: 147-154 (1992) may be employed.

Other gene delivery vehicles and methods may be employed; including polycationic condensed DNA linked or unlinked to kill adenovirus alone, for example Curiel, Hum. Gene Ther. 3: 147-154 (1992); ligand-linked DNA, for example see Wu, J. Biol. Chem. 264: 16985-16987 (1989); eukaryotic cell delivery vehicles cells, for example see U.S. Ser. No. 08/240,030, filed May 9, 1994, and U.S. Ser. No. 08/404,796; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell. Biol. 14:2411-2418 (1994), and in Woffendin, Proc. Natl. Acad. Sci. 91:1581-1585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Patent Publication Nos. WO 95/13 796, WO 94/23697, and WO 91/14445, and EP No. 0 524 968.

Further non-viral delivery suitable for use includes mechanical delivery systems such as the approach described in Woffendin et al., Proc. Natl. Acad. Sci. USA 91(24): 11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033.

The subject antibodies or antibody fragments may be conjugated directly or indirectly to effective moieties, e.g., radionuclides, toxins, chemotherapeutic agents, prodrugs, cytostatic agents, enzymes and the like. In a preferred embodiment the antibody or fragment will be attached to a therapeutic or diagnostic radiolabel directly or by use of a chelating agent. Examples of suitable radiolabels are well known and include ⁹⁰Y, ¹²⁵I, ¹³¹I, ¹¹¹I, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re.

Examples of suitable drugs that my be coupled to antibodies include methotrexate, adriamycine and lymphokines such as interferons, interleukins and the like. Suitable toxins which may be coupled include ricin, cholera and diptheria toxin.

In a preferred embodiment, the subject antibodies will be attached to a therapeutic radiolabel and used for radioimmunotherapy.

Inhibitory Oligonucleotides

In certain circumstances, it may be desirable to modulate or decrease the amount of the protein expressed by a colon cell. Thus, in another aspect of the present invention, inhibitory oligonucleotides can be made and a method utilized for diminishing the level of expression a colon antigen according to the invention by a cell comprising administering one or more inhibitory oligonucleotides, or a precursor thereof or a nucleic acid encoding the same. By inhibitory oligonucleotides reference is made to oligonucleotides that have a nucleotide sequence that interacts through base pairing with a specific complementary nucleic acid sequence involved in the expression of a target molecule, such that the expression of the gene is reduced. Preferably, the specific nucleic acid sequence involved in the expression of the gene is a genomic DNA molecule or mRNA molecule that encodes the gene. This genomic DNA molecule can comprise regulatory regions of the gene, or the coding sequence for the mature gene.

The term complementary to a nucleotide sequence in the context of inhibitory oligonucleotides and methods therefore means sufficiently complementary to such a sequence as to allow hybridization to that sequence in a cell, i.e., under physiological conditions. Antisense oligonucleotides preferably comprise a sequence containing from about 8 to about 100 nucleotides and more preferably the inhibitory oligonucleotides comprise from about 15 to about 30 nucleotides. They are typically single-stranded, and may be selected from antisense oligonucleotides, ribozymes, siRNAs, etc. Inhibitory oligonucleotides can also contain a variety of modifications that confer resistance to nucleolytic degradation such as, for example, modified internucleoside linages [Uhlmann and Peyman, Chemical Reviews 90:543-548 (1990); Schneider and Banner, Tetrahedron Lett. 31:335, (1990) which are incorporated by reference], modified nucleic acid bases as disclosed in U.S. Pat. No. 5,958,773 and patents disclosed therein, and/or sugars and the like.

Any modifications or variations of the inhibitory molecule which are known in the art to be broadly applicable to inhibitory technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The inhibitory compounds of the invention can include modified bases. The inhibitory oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. [Branch, A. D., T.I.B.S. 23:45-50 (1998)].

The therapeutic or pharmaceutical compositions of the present invention can be administered by any suitable route known in the art including for example intravenous, subcutaneous, intramuscular, transdermal, intrathecal or intracerebral. Administration can be either rapid as by injection or over a period of time as by slow infusion or administration of slow release formulation.

Additionally, the subject colon tumor proteins can also be linked or conjugated with agents that provide desirable pharmaceutical or pharmacodynamic properties. For example, the protein can be coupled to any substance known in the art to promote penetration or transport across the blood-brain barrier such as an antibody to the transferrin receptor, and administered by intravenous injection (see, for example, Friden et al., Science 259:373-377 (1993) which is incorporated by reference). Furthermore, the subject protein A or protein B can be stably linked to a polymer such as polyethylene glycol to obtain desirable properties of solubility, stability, half-life and other pharmaceutically advantageous properties. [See, for example, Davis et al., Enzyme Eng. 4:169-73 (1978); Buruham, Am. J. Hosp. Pharm. 51:210-218 (1994) which are incorporated by reference].

The compositions are usually employed in the form of pharmaceutical preparations. Such preparations are made in a manner well known in the pharmaceutical art. See, e.g. Remington Pharmaceutical Science, 18th Ed., Merck Publishing Co. Eastern PA, (1990). One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. The subject colon tumor antigens, fragments or variants thereof can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parental administration in either unit dosage or multi-dose form or for direct infusion into the cerebrospinal fluid by continuous or periodic infusion.

Dose administration can be repeated depending upon the pharmacokinetic parameters of the dosage formulation and the route of administration used.

It is also contemplated that certain formulations containing the subject antibody or nucleic acid antagonists are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and promote absorption such as, for example, surface active agents.

The specific dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In one embodiment of this invention, the protein may be therapeutically administered by implanting into patients vectors or cells capable of producing a biologically-active form of the protein or a precursor of protein, i.e., a molecule that can be readily converted to a biological-active form of the protein by the body. In one approach, cells that secrete the protein may be encapsulated into semipermeable membranes for implantation into a patient. The cells can be cells that normally express the protein or a precursor thereof or the cells can be transformed to express the protein or a precursor thereof. It is preferred that the cell be of human origin and that the protein be a human protein when the patient is human. However, it is anticipated that non-human primate homologues of the protein discussed infra may also be effective.

Detection of Subject Colon Proteins or Nucleic Acids

In a number of circumstances it would be desirable to determine the levels of protein or corresponding mRNA in a patient. Evidence disclosed infra suggests the subject colon proteins may be expressed at different levels during some diseases, e.g., cancers, provides the basis for the conclusion that the presence of these proteins serves a normal physiological function related to cell growth and survival. Endogenously produced protein according to the invention may also play a role in certain disease conditions.

The term “detection” as used herein in the context of detecting the presence of protein in a patient is intended to include the determining of the amount of protein or the ability to express an amount of protein in a patient, the estimation of prognosis in terms of probable outcome of a disease and prospect for recovery, the monitoring of the protein levels over a period of time as a measure of status of the condition, and the monitoring of protein levels for determining a preferred therapeutic regimen for the patient, e.g. one with colon cancer.

To detect the presence of a colon protein according to the invention in a patient, a sample is obtained from the patient. The sample can be a tissue biopsy sample or a sample of blood, plasma, serum, CSF, urine or the like. It has been found that the subject proteins are expressed at high levels in some cancers. Samples for detecting protein can be taken from colon tissues. When assessing peripheral levels of protein, it is preferred that the sample be a sample of blood, plasma or serum. When assessing the levels of protein in the central nervous system a preferred sample is a sample obtained from cerebrospinal fluid or neural tissue. The sample may be collected by various techniques known per se in the art, including non-invasive techniques, or may be obtained from sample collections.

In some instances, it is desirable to determine whether the gene is intact in the patient or in a tissue or cell line within the patient. By an intact gene, it is meant that there are no alterations in the gene such as point mutations, deletions, insertions, chromosomal breakage, chromosomal rearrangements and the like wherein such alteration might alter production of the corresponding protein or alter its biological activity, stability or the like to lead to disease processes. Thus, in one embodiment of the present invention a method is provided for detecting and characterizing any alterations in the gene. The method comprises providing an oligonucleotide that contains the gene, genomic DNA or a fragment thereof or a derivative thereof. By a derivative of an oligonucleotide, it is meant that the derived oligonucleotide is substantially the same as the sequence from which it is derived in that the derived sequence has sufficient sequence complementarily to the sequence from which it is derived to hybridize specifically to the gene. The derived nucleotide sequence is not necessarily physically derived from the nucleotide sequence, but may be generated in any manner including for example, chemical synthesis or DNA replication or reverse transcription or transcription.

Typically, patient genomic DNA is isolated from a cell sample from the patient and digested with one or more restriction endonucleases such as, for example, TaqI and AluI. Using the Southern blot protocol, which is well known in the art, this assay determines whether a patient or a particular tissue in a patient has an intact colon gene according to the invention or a gene abnormality.

Hybridization to a gene would involve denaturing the chromosomal DNA to obtain a single-stranded DNA; contacting the single-stranded DNA with a gene probe associated with the gene sequence; and identifying the hybridized DNA-probe to detect chromosomal DNA containing at least a portion of a gene.

The term “probe” as used herein refers to a structure comprised of a polynucleotide that forms a hybrid structure with a target sequence, due to complementarily of probe sequence with a sequence in the target region. Oligomers suitable for use as probes may contain a minimum of about 8-12 contiguous nucleotides which are complementary to the targeted sequence and preferably a minimum of about 20.

A gene according to the present invention can be DNA or RNA oligonucleotides and can be made by any method known in the art such as, for example, excision, transcription or chemical synthesis. Probes may be labeled with any detectable label known in the art such as, for example, radioactive or fluorescent labels or enzymatic marker. Labeling of the probe can be accomplished by any method known in the art such as by PCR, random priming, end labeling, nick translation or the like. One skilled in the art will also recognize that other methods not employing a labeled probe can be used to determine the hybridization. Examples of methods that can be used for detecting hybridization include Southern blotting, fluorescence in situ hybridization, and single-strand conformation polymorphism with PCR amplification.

Hybridization is typically carried out at 25°-45° C., more preferably at 32°-40° C. and more preferably at 37°-38° C. The time required for hybridization is from about 0.25 to about 96 hours, more preferably from about one to about 72 hours, and most preferably from about 4 to about 24 hours.

Gene abnormalities can also be detected by using the PCR method and primers that flank or lie within the gene. The PCR method is well known in the art. Briefly, this method is performed using two oligonucleotide primers which are capable of hybridizing to the nucleic acid sequences flanking a target sequence that lies within a gene and amplifying the target sequence. The terms “oligonucleotide primer” as used herein refers to a short strand of DNA or RNA ranging in length from about 8 to about 30 bases. The upstream and downstream primers are typically from about 20 to about 30 base pairs in length and hybridize to the flanking regions for replication of the nucleotide sequence. The polymerization is catalyzed by a DNA-polymerase in the presence of deoxynucleotide triphosphates or nucleotide analogs to produce double-stranded DNA molecules. The double strands are then separated by any denaturing method including physical, chemical or enzymatic. Commonly, a method of physical denaturation is used involving heating the nucleic acid, typically to temperatures from about 80° C. to 105° C. for times ranging from about 1 to about 10 minutes. The process is repeated for the desired number of cycles.

The primers are selected to be substantially complementary to the strand of DNA being amplified. Therefore, the primers need not reflect the exact sequence of the template, but must be sufficiently complementary to selectively hybridize with the strand being amplified.

After PCR amplification, the DNA sequence comprising the gene or a fragment thereof is then directly sequenced and analyzed by comparison of the sequence with the sequences disclosed herein to identify alterations which might change activity or expression levels or the like.

In another embodiment, a method for detecting a tumor protein according to the invention is provided based upon an analysis of tissue expressing the gene. Certain tissues such as colon tissues have been found to overexpress the subject gene. The method comprises hybridizing a polynucleotide to mRNA from a sample of tissue that normally expresses the gene. The sample is obtained from a patient suspected of having an abnormality in the gene.

To detect the presence of mRNA encoding the protein, a sample is obtained from a patient. The sample can be from blood or from a tissue biopsy sample. The sample may be treated to extract the nucleic acids contained therein. The resulting nucleic acid from the sample is subjected to gel electrophoresis or other size separation techniques.

The mRNA of the sample is contacted with a DNA sequence serving as a probe to form hybrid duplexes. The use of labeled probes as discussed above allows detection of the resulting duplex.

When using the cDNA encoding the protein or a derivative of the cDNA as a probe, high stringency conditions can be used in order to prevent false positives, that is, the hybridization and apparent detection of the gene nucleotide sequence when in fact an intact and functioning gene is not present. When using sequences derived from the gene cDNA, less stringent conditions could be used, however, this would be a less preferred approach because of the likelihood of false positives. The stringency of hybridization is determined by a number of factors during hybridization and during the washing procedure, including temperature, ionic strength, length of time and concentration of formamide. These factors are outlined in, for example, Sambrook et al. [Sambrook et al. (1989), supra].

In order to increase the sensitivity of the detection in a sample of mRNA encoding the protein A or protein B, the technique of reverse transcription/polymerization chain reaction (RT/PCR) can be used to amplify cDNA transcribed from mRNA encoding the colon tumor antigen. The method of RT/PCR is well known in the art, and can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end primer. Typically, the primer contains an oligo(dT) sequence. The cDNA thus produced is then amplified using the PCR method and gene A or gene B specific primers. [Belyavsky et al., Nucl. Acid Res. 17:2919-2932 (1989); Krug and Berger, Methods in Enzymology, 152:316-325, Academic Press, NY (1987) which are incorporated by reference].

The polymerase chain reaction method is performed as described above using two oligonucleotide primers that are substantially complementary to the two flanking regions of the DNA segment to be amplified. Following amplification, the PCR product is then electrophoresed and detected by ethidium bromide staining or by phosphoimaging.

The present invention further provides for methods to detect the presence of the protein in a sample obtained from a patient. Any method known in the art for detecting proteins can be used. Such methods include, but are not limited to immunodiffusion, immunoelectrophoresis, immunochemical methods, binder-ligand assays, immunohistochemical techniques, agglutination and complement assays. [Basic and Clinical Immunology, 217-262, Sites and Terr, eds., Appleton & Lange, Norwalk, Conn., (1991), which is incorporated by reference]. Preferred are binder-ligand immunoassay methods including reacting antibodies with an epitope or epitopes of the colon tumor antigen protein and competitively displacing a labeled colon antigen according to the invention or derivative thereof.

As used herein, a derivative of the subject colon tumor antigen is intended to include a polypeptide in which certain amino acids have been deleted or replaced or changed to modified or unusual amino acids wherein the derivative is biologically equivalent to gene and wherein the polypeptide derivative cross-reacts with antibodies raised against the protein. By cross-reaction it is meant that an antibody reacts with an antigen other than the one that induced its formation.

Numerous competitive and non-competitive protein binding immunoassays are well known in the art. Antibodies employed in such assays may be unlabeled, for example as used in agglutination tests, or labeled for use in a wide variety of assay methods. Labels that can be used include radionuclides, enzymes, fluorescent tags, chemiluminescers, enzyme substrates or co-factors, enzyme inhibitors, particles, dyes and the like for use in radioimmunoassay (RIA), enzyme immunoassays, e.g., enzyme-linked immunosorbent assay (ELISA), fluorescent immunoassays and the like.

A further aspect of this invention relates to a method for selecting, identifying, screening, characterizing or optimizing biologically active compounds, comprising a determination of whether a candidate compound binds, preferably selectively, a target molecule as disclosed above. Such target molecules include nucleic acid sequences, polypeptides and fragments thereof, typically colon-specific antigens, even more preferably extracellular portions thereof. Binding may be assessed in vitro or in vivo, typically in vitro, in cell based or accellular systems. Typically, the target molecule is contacted with the candidate compound in any appropriate device, and the formation of a complex is determined. The target molecule and/or the candidate compound may be immobilized on a support. The compounds identified or selected represent drug candidates or leads for treating cancer diseases, particularly colon cancer.

While the invention has been described supra, including preferred embodiments, the following examples are provided to further illustrate the invention.

### EXAMPLE

### Tissue Sources

Appropriate patient samples were obtained with relevant clinical parameters, and patient consent. Histological assessment was performed on all samples and diagnosis by pathology confirmed the presence and/or absence of malignancy within each sample. Clinical data generally included patient history, physiopathology, and parameters relating to colon cancer physiology. The research specimens were divided into two groups; early-stage CRC (Dukes' stage A or B) and late-stage CRC (Dukes' stage C or D). Eight matched sets containing normal and malignant samples were obtained for each group, resulting in a total of 32 specimens. Two matched pairs from each group were used for the construction of DATAS™ libraries, the remaining samples were used for expression profiling studies by RT-PCR.

### Quality Assessment of Tissue Samples

Six patient samples were purchased from Integrated Laboratory Services (ILS-Bio). Each patient sample contains a matched pair of normal colon tissue and colon tumor that were obtained during surgery. RNA was isolated from each sample using Trizol and was inspected for quality control following isolation using an Agilent 2100 analyzer. One of the late stage samples was degraded, and this sample was removed from consideration and the analysis was performed with the two remaining samples. To maintain continuity between the comparisons of the early and late stage samples, two samples were used to construct the early-stage analysis. The samples were subjected to a patented process, DATAS™ (U.S. Pat. No. 6,251,590), that uses molecular biology techniques to provide information on a alternative RNA Splicing deregulations associated with diseases, colon cancer in this case. Two DATAS™ libraries were constructed, one from the early stage samples, and one from the late stage samples.

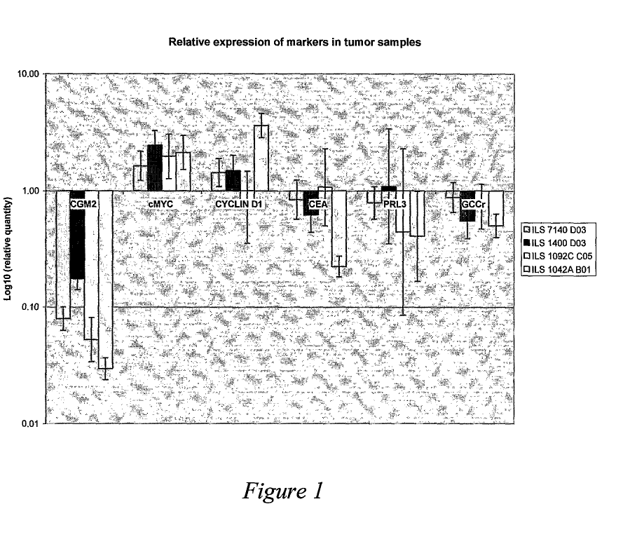

The selection of the two samples was based on qPCR analysis with markers for CRC that were identified in the literature (table 1). As part of this analysis we examined these markers using both end point RT-PCR and qPCR on the tissue RNA samples. Samples 7140 and 1400 were selected based on their qPCR results for marker CGM2 (see FIG. 1). This gene is a member of the carcinoembryonic antigen family and has been shown to be down-regulated at an early stage in the progression colorectal cancer. The qPCR data for this marker displayed an appropriate trend with the late-stage samples showing a greater extent of down-regulation relative to the early stage. The qPCR data for early-stage sample, #1481, resembled the late-stage samples and was not included in the DATAS™ library construction to maximize the differences between the early- and late-stage libraries (data not shown).

A comparison of the qPCR data for each of the late- and early-stage samples used in constructing the DATAS™ libraries against this panel of markers is presented in FIG. 1

### Generation of the DATAS™ Library

Samples were selected based on their expression of tissue markers (normal vs. tumor). Total RNA of 100 ug of each sample was used to construct the DATAS™ libraries as previously disclosed in U.S. Pat. No. 6,251,590, the disclosure of which is incorporated by reference in its entirety. Briefly, total RNA was isolated from the normal and tumor selected samples and mRNA was subsequently purified from the total RNA for each sample. Synthesis of cDNA was performed using a biotinylated oligo (dT) primer. The biotinylated cDNA was hybridized with the mRNA of the opposite sample to form heteroduplexes between the cDNA and the mRNA. For example, the biotinylated cDNA of the normal colon sample was hybridized with colon tumor mRNA. Similarly, colon tumor biotinylated cDNA was hybridized with colon normal RNA to generate the second DATAS™ library. Streptavidin coated beads were used to purify the complexes by binding the biotin present on the cDNA. The heteroduplexes were digested with RNAse H to degrade the RNA that was complementary to the cDNA. All mRNA sequences that were different from the cDNA remained intact. These single stranded RNA fragments or “loops” were subsequently amplified with degenerate primers and cloned into either pGEM-T or pCR II TOPO vector (Invitrogen) to produce the DATAS™ library.

### Clone Sequencing and Bioinformatics Analysis:

E. coli was transformed with the DATAS™ library for the isolation of individual clones using standard molecular biology techniques. From these libraries, 9,600 individual clones were isolated and sequenced using an automated Applied Biosystems 3100 sequencer. The nucleotide sequences that were obtained were submitted to ExonHit's proprietary bioinformatics pipeline for analysis. As the DATAS™ library is prepared with PCR amplified DNA, many copies of the same sequence are present in the clones isolated from the libraries. Therefore it is important to reduce the redundancy of the clones to identify the number of unique, nonrepeating sequences that are isolated. From this large set of DATAS™ fragments, 1709 unique, nonredundant sequences were identified and each DATAS™ fragment was annotated with a candidate gene.

The annotation was performed by aligning the DATAS™ fragment to the human genome sequenceusing proprietary annotation algorithms. Each DATAS™ fragment sequence was annotated with a corresponding gene that overlapped the genomic sequence containing the DATAST fragment. 1467 genes were annotated with either the Refseq accession number, or a hypothetical gene prediction from different algorithms, for example, Genscan, Twinscan, or Fgenesh++, while 242 DATAS™ fragments that matched the genome had no identified overlapping gene. Identified genes were either matched to the sequence of the DATAS™ fragment (in case of exon to fragment match), or overlapped with the DATAS™ fragment (in case of intron to fragment match), and the full length sequence of the gene was identified. These sequences were further analyzed to detect all potential secreted and membrane spanning proteins. Membrane and secreted proteins were predicted through the use of different algorithms commercially available. For example, TMHMM and SignalP (CBS) were used to identify membrane-spanning domains and signal peptide sequences, respectively, present within the amino acid sequence of the candidate gene. DATAS™ fragments were located within the sequence in an attempt to determine whether the spliced event affected intracellular or extracelullar domains for the transmembrane proteins. Genes associated with the sequence were ranked in order to maximize the identification of successful diagnostic and therapeutic targets. The highest priority genes had characteristics where the gene was a known or predicted membrane secreted protein, the function of the gene was known, and the DATAS™ fragment mapped to an intron. In addition, DATAS™ fragments mapping to the extracellular domain of the protein, indicating that the DATAS™ fragment would be presented outside the cell, and secreted proteins were considered the most viable candidates.

Based on the bioinformatic analysis, clones were prioritized in three groups:
A) Genes encoding known secreted proteins with DATAS™ fragments located in introns in the secreted proteins.
B) Genes encoding known and predicted secreted proteins with DATAS™ fragments matching an exon and the neighboring intron, indicating an exon extension.
C) Genes encoding known secreted proteins with DATAS™ fragments matching known exons.
   Fragments were annotated for the gene name, whether the gene had a positive signal for a transmembrane region or a signal secretory sequence, and the type of event that was identified; a novel event that matched a portion of an intron, or a fragment that matched an exon and overlapped with the neighboring intron, suggesting an exon extension. Seventy-three (73) events were found to be completely novel events, and fifty (50) events suggested an exon extension. Reference genes were captured as known gene sequence (SEQ ID NOS. 45, 50, 60, 63, 68, 74, 80, 86, 92, 93, and 95), with the corresponding known protein sequence (SEQ ID NOS. 46, 51, 61, 64, 75, 81, 87, and 94). This information was used later in the process to identify novel exons, exon extensions, and novel epitopes in the protein structure.

In addition to the candidates identified through DATAS™ experimental methods, a set of candidate genes were examined for novel, alternatively spliced isoforms that would be synthesized in pathological conditions. Potential splice events were first identified through a proprietary bioinformatic method that uses public and private Expressed Sequence Tags (EST's), and aligns them to the gene of interest, looking for differences that would indicate an alternatively spliced isoform exists. Oligonucleotide primers were designed to this event and the subsequent amplicon produced was sequenced to verify the structure of the RNA. The “bioinformatically derived sequence” entries are SEQ ID NOS. 73, 79, 85, and 91. In addition, multiple sets of oligonucleotides were designed to capture novel events that were not indicated through the bioinformatics process. These potentially novel isoforms were treated similarly as the experimentally identified isoforms below.

### Expression Monitoring:

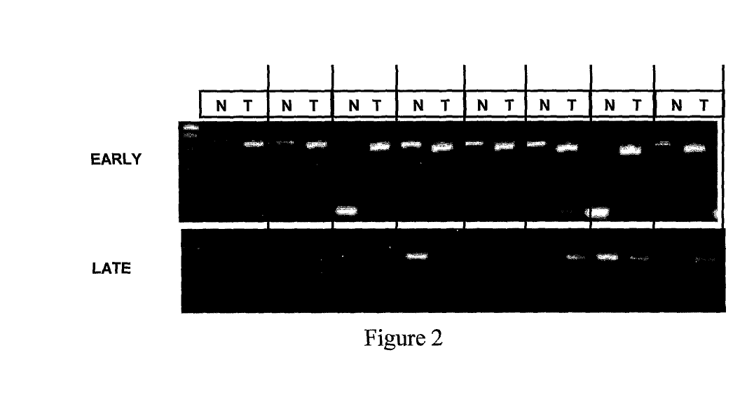

A valid target for colon cancer requires that its expression be differentially expressed in tumor sample compared to the normal tissue. Assessment of the expression profile for each prioritized sequence was performed by RT-PCR, a procedure well known in the art. A protocol known as touchdown PCR was used, described in the user's manual for the GeneAmp PCR system 9700, Applied Biosystems. Briefly, PCR primers were designed to the DATAS™ fragment, or the bioinformatically identified event, and used for end point RT-PCR analysis. Each RT reaction contained 5 ug of total RNA and was performed in a 100 ul volume using Archive RT Kit (Applied Biosystems). The RT reactions were diluted 1:50 with water and 4 ul of the diluted stock was used in a 50 ul PCR reaction consisting of one cycle at 94° C. for 3 min, 5 cycles at 94° C. for 30 seconds, 60° C. for 30 seconds and 72° C. for 45 seconds, with each cycle reducing the annealing temperature by 0.5 degree. This was followed by 30 cycles at 94° C. for 30 seconds, 55° C. for 30 seconds, and 72° C. for 45 seconds. 15 ul was removed from each reaction for analysis and the reactions were allowed to proceed for an additional 10 cycles. This produced reactions for analysis at 30 and 40 cycles, and allowed the detection of differences in expression where the 40 cycle reactions had saturated. Total RNA was used for all reactions. Expression profiles were generated using matched samples for early stage tumors (8 normal and 8 tumor) and late stage tumors (8 normal and 8 tumor). Profiles that showed a differential expression between normal and tumor are summarized in Table 2. An example of the generated expression profiles is illustrated in FIG. 2, where the differential expression of the novel event is clearly upregulated in tumor samples when compared to the matched control sample. The score for this clone, SEQ ID NO: 28, with the early stage samples is 8.0, while the score for the late stage samples is 3.3. The score reflects the differential expression of this event in tumor samples versus the matching normal colon tissue from the same patient. The early stage set contains samples obtained from patients with confirmed Dukes' A or B stage cancer, and the late stage set contains samples from patients with confirmed Dukes' C. or D stage cancers. The values are reported where the whole number indicates the number of samples that showed an up regulation in the tumor, and the tenths decimal indicates how many samples showed a down regulation with respect to the tumor (i.e. 3.3 would indicate three samples show an upregulation of the splice event in tumor compared to the matched control, and three samples showed a down regulation in tumor compared to matched controls, while an 8.0 indicates eight (8) samples show an upregulation of the splice event in tumor compared to the matched control, and no (zero) samples showed a down regulation in tumor compared to matched controls. Forty four fragments isolated from DATAS™ were found to be differentially expressed in colon tumor samples as compared to normal colon tissue. These sequences are presented in SEQ ID NOS. 1-44. Bioinformatically derived sequence information were either differentially expressed or novel coding sequence was identified to be expressed in tumors (see below).

In addition, while performing these expression profile assays, we identified that SEQ ID NO: 74 (transcobalamin I), corresponding to the wild-type isoform of SEQ ID NO: 73 was differentially expressed between tumor and normal colon tissues with a score of 6.0 for early and 8.0 for late samples (FIG. 2B).

### Verification of RNA Structure:

DATAS™ identifies sequences that are altered between the experimental samples. However, the exact sequence of the junctions or borders that the DATAS™ fragment represents sometimes needs to be further characterized. The DATAS™ fragment was used, however, to design experiments that refine the sequence of splice event, provide the exact splice sites used, and the sequence of the coding region was identified experimentally. Primers were designed to amplify a region of the gene larger than the proposed DATAS™ fragment sequence. A similar approach was used for the bioinformatically derived gene set to identify the splice event and its junctions.

These amplicons were subsequently cloned and sequenced for the identification of the exact junctions of all exons and introns in order to identify the splice sites. This required partial cloning of the isoforms from an identified sample to verify the primary structure (sequence) of the isoforms. RNA samples obtained from three individuals were used as the starting material for RT-PCR amplifications. Each reaction was run in duplicate and the products from each reaction were sub-cloned and sequenced. A consensus sequence was obtained by combining the sequencing results from the six separate reactions. Four samples (2 normal, 2 early tumor) were used for the verification of the mRNA structure of the prioritized genes.

The confirmed structure and sequence of the clones are found in SEQ ID NOS. 52, 56, 62, 73, 79, 85, and 91. Once the event was identified, the novel nucleic acid sequence that was captured in the amplicons was translated to generate the novel protein sequence of the isoform. The novel gene (nucleic acid) sequences are listed in SEQ ID NOS. 47, 53, 57, 65, 70, 76, 82, 88, and 96. These novel protein sequences are listed in SEQ ID NOS. 48, 54, 58, 66, 71, 77, 83, 89, and 97. Comparisons of the novel protein isoform with the known proteins structure (see above) generated significant differences in amino acid content. This difference was chosen as the target for antibody generation for the detection of the novel protein isoform in tissue or serum. These novel epitopes are listed in SEQ ID NOS. 49, 55, 59, 67, 72, 78, 84, 90, 98 and 99.

### Isolation of Full-Length Clones of Isoforms:

Isolation of the full-length clones containing both isoforms was accomplished utilizing the sequence information and DNA fragments generated during the structure validation process. Several methods are applicable to isolation of the full length clone. Where full sequence information regarding the coding sequence is available, gene specific primers were designed from the sequence and used to amplify the coding sequence directly from the total RNA of the tissue samples. An RT-PCR reaction was set up using these gene specific primers. The RT reaction was performed as described infra, using oligo dT to prime for cDNA. Second strand was produced by standard methods to produce double stranded cDNA. PCR amplification of the gene was accomplished using gene specific primers. PCR consisted of 30 cycles at 94° C. for 30 seconds, 55° C. for 30 seconds, and 72° C. for 45 seconds. The reaction products were analyzed on 1% agarose gels and the amplicons were ligated into prepared vectors with A overhangs for amplicon cloning. 1 ul of the ligation mixture was used to transform E. coli for cloning and isolation of the amplicon. Once purified, the plasmid containing the amplicon was sequenced on an ABI 3100 automated sequencer.

Where limited sequence information was available, 3′ and 5′ RACE was utilized. Briefly, gene-specific oligonucleotides were designed based on the DATAS™ fragment. The oligonucleotides were used for extension using total RNA from normal colon and colon tumor tissue following the procedures of Sambrook et al (1989). The eluted cDNA was converted to double strand plasmid DNA and used to transform E. coli cells and the longest cDNA clone was subjected to DNA sequencing. Full length clones were also obtained using sequence specific primers and following the recommended procedures for the First Choice® RLM_RACE kit produced by Ambion, Inc. (Austin, Tex.) using either single patient or pooled RNA samples. Additionally, 3′ RACE was performed when additional sequence information was required for designing sequence-specific oligonucleotides. The full-length clones produced in this manner were sequenced in their entirety to verify their nucleic acid sequence and composition.

### REFERENCES

Alcaraz et al., Cancer Res., 55:3998-4002, 1994.
Allhoff et al., World J. Urol., 7:12-16, 1989.
American Cancer Society. Cancer Facts and Figures 2002. 2002.
An et al., Proc. Amer. Assn. Canc. Res., 36:82, 1995.
An et al., Molec. Urol., 2: 305-309, 1998.
Antibodies: A Laboratory Manual, Cold Spring Harbor Laboratory, Cold Spring Harbor Press, Cold Spring Harbor, N.Y., 1988.
Babian et al., J. Urol., 156:432-437, 1996.
Badalament et al., J. Urol., 156:1375-1380, 1996.
Baichwal and Sugden, In: Gene Transfer, Kucherlapati (Ed.), Plenum Press, New York, pp 117-148, 1986.
Bangharn et al., J. Mol. Biol. 13: 238-252, 1965.
Barinaga, Science, 271: 1233, 1996.
Bedzyk et al., J. Biol. Chem., 265:18615, 1990
Bell et al., “Gynecological and Genitourinary Tumors,” In: Diagnostic Immunopathology, Colvin, Bhan and McCluskey (Eds.), 2nd edition, Ch. 31, Raven Press, New York, pp 579-597, 1995.
Bellus, J. Macromol. Sci. Pure Appl. Chem., A31(1):1355-1376, 1994.
Benvenisty and Neshif, Proc. Nat. Acad. Sci. USA, 83:9551-9555, 1986.
Berggren et al., Arch Biochem Biophys., 389(1): p. 144-9, 2001.
Bittner et al., Methods in Enzymol, 153:516-544, 1987.
Bookstein et al., Science, 247:712-715, 1990a.
Bookstein et al., Proc. Nat'l Acad. Sci. USA, 87:7762-7767, 1990b.
Bova et al., Cancer Res., 53:3869-3873, 1993
Brawn et al., The Colon, 28:295-299, 1996.
Campbell, In: Monoclonal Antibody Technology, Laboratory Techniques in Biochemistry and Molecular Biology, Burden and Von Knippenberg (Eds.), Vol. 13:75-83, Elsevier, Amsterdam, 1984.
Capaldi et al., Biochem. Biophys. Res. Comm, 76:425, 1977.
Carter and Coffey, In: Colon Cancer: The Second Tokyo Symposium, J. P. Karr and H. Yamanak (Eds.), Elsevier, New York, pp 19-27, 1989.
Carter and Coffey, Colon, 16:3948, 1990.
Carter et al., Proc. Nat'l Acad. Sci. USA, 87:8751-8755, 1990.
Carter et al., Proc. Nat'l Acad. Sci. USA, 93: 749-753, 1996.
Carter et al., J. Urol., 157:2206-2209, 1997.
Cech et al., Cell, 27:487-496, 1981.
Chang et al., Hepatology, 14: 124A, 1991.
Chaudhary et al., Proc. Nat'l Acad. Sci., 87:9491, 1990
Chen and Okayama, MoL Cell. Biol., 7:2745-2752, 1987.
Chen et al., Clin. Chem., 41:273-282, 1995a.
Chen et al., Proc. Am. Urol. Assn, 153:267 A, 1995.
Chinault and Carbon, “Overlap Hybridization Screening: Isolation and Characterization of Overlapping DNA Fragments Surrounding the LEU2 Gene on Yeast Chromosome III,” Gene, 5:111-126, 1979.
Chomczynski and Sacchi, Anal. Biochem., 162:156-159, 1987.
Christensson et al., J. Urol., 150:100-105, 1993.
Coffin, In: Virology, Fields et al. (Eds.), Raven Press, New York, pp 1437-1500, 1990.
Colberre-Garapin et al., J. Mol. Biol., 150:1, 1981.
Colvin et al., Diagnostic Immunopathology, 2nd edition, Raven Press, New York, 1995.
Cooner et al., J. Urol., 143:1146-1154, 1990.
Couch et al., Am. Rev. Resp. Dis., 88:394-403, 1963.
Coupar et al., Gene, 68:1-10, 1988.
Culver et al., Science, 256:1550-1552, 1992.
Davey et al., EPO No. 329 822.
Deamer and Uster, “Liposome Preparation: Methods and Mechanisms,” In: Liposomes, M. Ostro (Ed.), 1983.
Diamond et al., J. Urol., 128:729-734, 1982.
Donahue et al., J. Biol. Chem., 269:8604-8609, 1994.
Dong et al., Science, 268:884-886, 1995.
Dubensky et al., Proc. Nat. Acad. Sci. USA, 81:7529-7533, 1984.
Dumont et al., J. Immunol., 152:992-1003, 1994.
Elledge et al., Cancer Res. 54:3752-3757, 1994
European Patent Application EPO No. 320 308
Fearon et al., Science, 247:47-56, 1990.
Fechheirner et al., Proc. Natl. Acad. Sci. USA, 84:8463-8467, 1987.
Forster and Symons, Cell, 49:211-220, 1987.
Fraley et al., Proc. Natl. Acad. Sci. USA, 76:3348-3352, 1979.
Friedmann, Science, 244:1275-1281, 1989.
Freifelder, In: Physical Biochemistry Applications to Biochemistry and Molecular Biology, 2nd ed., Wm. Freeman and Co., New York, N.Y., 1982.
Frohman, In: PCR Protocols: A Guide to Methods and Applications, Academic Press, New York, 1990.
Gefter et al., Somatic Cell Genet., 3:231-236, 1977.
Gerlach et al., Nature (London), 328:802-805, 1987.
Ghosh-Choudhury et al., EMBO J., 6:1733-1739, 1987.
Gingeras et al., PCT Application WO 88/10315.
Ghosh and Bachhawat, In: Liver Diseases, Targeted Diagnosis and Therapy Using Specific Receptors and Ligands, Wu et al. (Eds.), Marcel Dekker, New York, pp 87-104, 1991.
Goding, In: Monoclonal Antibodies: Principles and Practice, 2nd ed., Academic Press, Orlando, Fla., pp 60-61, 65-66, 71-74, 1986.
Gomez-Foix et al., J. Biol. Chem., 267:25129-25134, 1992.
Gopal, Mol. Cell. Biol., 5:1188-1190, 1985.
Graham et al., J. Gen. Virol., 36:59-72, 1977.
Graham and van der Eb, Virology, 52:456-467, 1973.
Graham and Prevec, In: Methods in Molecular Biology: Gene Transfer and Expression Protocols 7, E. J. Murray (Ed.), Humana Press, Clifton, N.J., pp 205-225, 1991.
Gregoriadis (ed.), In: Drug Carriers in Biology and Medicine, pp 287-341, 1979.
Grunhaus and Horwitz, Sem. Virol., 3:237-252, 1992.
Hardcastle et al., Lancet; 348(9040):1472-1477, 1996
Harland and Weintraub, J. Cell Biol., 101: 1094-1099, 1985.
Harris et al., J. Urol., 157:1740-1743, 1997.
Heng et al., Proc. Nat. Acad. Sci. USA, 89: 9509-9513, 1992.
Hermonat and Muzycska, Proc. Nat. Acad. Sci. USA, 81:6466-6470, 1984.
Hersdorffer et al., DNA Cell Biol., 9:713-723, 1990.
Herz and Gerard, Proc. Natl. Acad. Sci. USA, 90:2812-2816, 1993.
Hess et al., J. Adv. Enzyme Reg., 7:149, 1968.
Hitzeman et al., J. Biol. Chem., 255:2073, 1980.
Holland et al., Biochemistry, 17:4900, 1978.
Horoszewicz, Kawinski and Murphy, Anticancer Res., 7:927-936, 1987.
Horwich, et al., J. Virol., 64:642-650, 1990.
Huang et al., Colon, 23: 201-212, 1993.
Innis et al., In: PCR Protocols, Academic Press, Inc., San Diego Calif., 1990.
Inouye et al., Nucl. Acids Res., 13:3101-3109, 1985.
Isaacs et al., Cancer Res., 51:4716-4720, 1991.
Isaacs et al., Sem. Oncol., 21:1-18, 1994.
Israeli et al., Cancer Res., 54:1807-1811, 1994.
Jacobson et al., JAMA, 274:1445-1449, 1995.
Johnson et al., In: Biotechnology and Pharmacy, Pezzuto et al., (Eds.), Chapman and Hall, New York, 1993.
Jones, Genetics, 85:12, 1977.
Jones and Shenk, Cell, 13:181-188, 1978.
Joyce, Nature, 338:217-244, 1989.
Kan et al., Genome Res 11, p. 889-900, 2001.
Kaneda et al., Science, 243:375-378, 1989.
Kato et al., J. Biol. Chem., 266:3361-3364, 1991.
Kim and Cech, Proc. Natl. Acad. Sci. USA, 84:8788-8792, 1987.
Kingsman et al., Gene, 7:141, 1979.
Klein et al., Nature, 327:70-73, 1987.
Kohler and Milstein, Nature, 256:495-497, 1975.
Kohler and Milstein, Eur. J. Immunol., 6:511-519, 1976.
Kronborg et al., Lancet; 348(9040):1467-1471, 1996
Kwoh et al., Proc. Nat. Acad. Sci. USA, 86:1173, 1989.
Landis et al., CA Cancer J. Clin., 48: 6-29, 1998.
Le Gal La Salle et al., Science, 259:988-990, 1993.
Levrero et al., Gene, 10 1: 195-202, 1991.
Liang and Pardee, Science, 257:967-971, 1992.
Liang and Pardee, U.S. Pat. No. 5,262,311, 1993.
Liang et al., Cancer Res., 52:6966-6968, 1992.
Lifton, Science, 272:676, 1996.
Lilja et al., Clin. Chem., 37:1618-1625, 1991.
Lithrup et al., Cancer, 74:3146-3150, 1994.
Lowy et al., Cell, 22:817, 1980.
Lukas et al., Cancer Res., 61(7): p. 3212-9, 2001.
Macoska et al., Cancer Res., 54:3824-3830, 1994.
Mandel et al., N Engl J Med; 328(19):1365-1371. 1993
Mandel et al., N Engl J Med; 343(22):1603-1607. 2000.
Mann et al., Cell, 33:153-159, 1983.
Markowitz et al., J. Virol., 62:1120-1124, 1988.
Marley et al., Urology, 48(6A): 16-22, 1996.
McCormack et al., Urology, 45:729-744, 1995.
Michel and Westhof, J. Mol. Biol. 216:585-610, 1990.
Midgley and Kerr, Lancet; 353(9150):391-3991999
Miki et al., Science, 266:66-71, 1994.
Milech et al., Genes Chromosomes Cancer., 32(3): p. 275-80, 2001.
Miller et al., PCT Application, WO 89/06700.
Modrek et al., Nucleic Acids Res 29, p. 2850-2859, 2001.
Mok et al., Gynecol. Oncol., 52:247-252, 1994.
Morahan et al., Science 272:1811, 1996.
Mulligan et al., Proc. Nat'l Acad. Sci. USA, 78:2072, 1981.
Mulligan, Science, 260:926-932, 1993.
Murphy et al., Cancer, 78: 809-818, 1996.
Murphy et al., Colon, 26:164-168, 1995.
Nakamura et al., In: Handbook of Experimental Immunology, (4th Ed.), Weir, E., Herzenberg, L. A., Blackwell, C., Herzenberg, L. (Eds.), Vol. 1, Chapter 27, Blackwell Scientific Publ., Oxford, 1987.
Nicolas and Rubinstein, In: Vectors: A Survey of Molecular Cloning Vectors and Their Uses, Rodriguez and Denhardt (Eds.), Butterworth, Stoneham, p 494-513, 1988.
Nicolau and Sene, Biochim. Biophys. Acta, 721:185-190, 1982.
Obermair et al., Gynecol Oncol., 83(2): p. 343-7, 2001.
O'Dowd et al., J. Urol., 158:687-698, 1997.
O'Hare et al., Proc. Nat'l Acad. Sci. USA, 78:1527, 1981.
Oesterling et al., J. Urol., 154:1090-1095, 1995.
Ohara et al., Proc. Nat'l Acad. Sci. USA, 86:5673-5677, 1989.
Orozco et al., Urology, 51:186-195, 1998.
Parker et al., CA Cancer J. Clin., 65:5-27, 1996.
Partin and Oesterling, Urology, 48 (6A):1-3, 1996.
Partin and Oesterling, J. Urol., 152:1358-1368, 1994.
Partin and Oesterling (Eds.), Urology, 48(6A) Supplement:1-87, 1996.
Paskind et al., Virology, 67:242-248, 1975.
PCT Application No. PCT/US87/00880
Pettersson et al., Clin. Chem., 41(10):1480-1488, 1995.
Piironen et al., Clin. Chem. 42:1034-1041, 1996.
Poola and Speirs, J Steroid Biochem Mol Biol., 78(5): p. 459-69, 2001.
Potter et al., Proc. Nat. Acad. Sci. USA, 81:7161-7165, 1984.
Racher et al., Biotechnology Techniques, 9:169-174, 1995.
Ragot et al., Nature, 361:647-650, 1993.
Ralph and Veltri, Advanced Laboratory, 6:51-56, 1997.
Ralph et al., Proc. Natl. Acad. Sci. USA, 90(22):10710-10714, 1993.
Reinhold-Hurek and Shub, Nature, 357:173-176, 1992.
Renan, Radiother. Oncol., 19:197-218, 1990.
Ribas de Pouplana and Fothergill-Gilmore, Biochemistry, 33:7047-7055, 1994.
Rich et al., Hum. Gene Ther., 4:461-476, 1993.
Ridgeway, In: Vectors: A Survey of Molecular Cloning Vectors and Their Uses, Rodriguez R L, Denhardt D T (Eds.), Butterworth, Stoneham, pp 467-492, 1988.
Rippe et al., Mol. Cell. Biol., 10:689-695, 1990.
Rosenfeld et al., Science, 252:431-434, 1991.
Rosenfeld et al., Cell, 68:143-155, 1992.
Roux et al., Proc. Nat'l Acad. Sci. USA, 86:9079-9083, 1989.
Sager et al., FASEB J., 7:964-970, 1993.
Sambrook et al., (ed.), In: Molecular Cloning, Cold Spring Harbor Laboratory Press, Cold Spring Harbor, N.Y., 1989.
Santerre et al., Gene, 30: 147-156, 1984.
Sarver, et al., Science, 247:1222-1225, 1990.
Scanlon et al., Proc Natl Acad Sci USA, 88:10591-10595, 1991.
Sidransky et al., Science, 252:706-709, 1991.
Sidransky et al., Cancer Res., 52:2984-2986, 1992.
Silver et al., Clin. Cancer Res., 3:81-85, 1997.
Slamon et al., Science, 224:256-262, 1984.
Slamon et al., Science, 235:177-182, 1987.
Slamon et al., Science, 244:707-712, 1989.
Smith, U.S. Pat. No. 4,215,051.
Smith et al., CA Cancer J Clin, 51(1):38-75, 2001
Soh et al., J. Urol., 157:2212-2218, 1997.
Stenman et al., Cancer Res., 51:222-226, 1991.
Stinchcomb et al., Nature, 282:39, 1979.
Stratford-Perricaudet and Perricaudet, In: Human Gene Transfer, O. Cohen-Haguenauer et al., (Eds.), John Libbey Eurotext, France, pp 51-61, 1991.
Stratford-Perricaudet et al., Hum. Gene. Ther., 1:241-256, 1990.
Sun and Cohen, Gene, 137:127-132, 1993.
Szoka and Papahadjopoulos, Proc. Nat'l. Acad. Sci. USA, 75: 4194-4198, 1978.
Szybalska et al., Proc. Nat'l Acad. Sci. USA, 48:2026, 1962.
Takahashi et al., Cancer Res., 54:3574-3579, 1994.
Taparowsky et al., Nature, 300:762-764, 1982.
Temin, In: Gene Transfer, Kucherlapati R. (Ed.), Plenum Press, New York, pp 149-188: 1986.
Tooze, In: Molecular Biology of DNA Tumor Viruses, 2nd ed., Cold Spring Harbor Laboratory, Cold Spring Harbor, N.Y., 1991.
Top et al., J. Infect. Dis., 124:155-160, 1971.
Tschemper et al., Gene, 10:157, 1980.
Tur-Kaspaet al., Mol. Cell. Biol., 6:716-718, 1986.
U.S. patent application Ser. No. 08/692,787
U.S. Pat. No. 4,196,265
U.S. Pat. No. 4,215,051
U.S. Pat. No. 4,683,195
U.S. Pat. No. 4,683,202
U.S. Pat. No. 4,800,159
U.S. Pat. No. 4,883,750
U.S. Pat. No. 5,354,855
U.S. Pat. No. 5,359,046
U.S. Pat. No. 6,251,590
Varmus et al., Cell, 25:23-36, 1981.
Veltri et al., J. Cell Biochem., 19(suppl):249-258, 1994.
Veltri et al., Urology, 48: 685-691, 1996.
Veltri et al., Sem. Urol. Oncol., 16:106-117, 1998.
Veltri et al., Urology, 53:139-147, 1999.
Visakorpi et al., Am. J. Pathol., 145:1-7, 1994.
Vogelstein et al., N Engl J. Med., 319(9):525-532. 1988
Wagner et al., Science, 260:1510-1513, 1993.
Walker et al., Proc. Nat'l Acad. Sci. USA, 89:392-396, 1992.
Watson et al., Cancer Res., 54:4598-4602, 1994.
Welsh et al., Nucl. Acids Res., 20:4965-4970, 1992.
Wigler et at, Cell, 11:223, 1977.
Wigler et al., Proc. Nat'l Acad. Sci. USA, 77:3567, 1980.
Wingo et al., CA Cancer J. Clin., 47: 239-242, 1997.
WO 90/07641, filed Dec. 21, 1990.
Wong et al., Int. J. Oncol., 3:13-17, 1993.
Wu and Wu, J. Biol. Chem., 262: 4429-4432, 1987.
Wu and Wu, Biochemistry, 27: 887-892, 1988.
Wu and Wu, Adv. Drug Delivery Rev., 12: 159-167, 1993.
Wu et al., Genomics, 4:560, 1989.
Yang et al., Proc. Natl. Acad. Sci. USA, 87:9568-9572, 1990.
Yokoda et al., Cancer Res. 52, 3402-3408, 1992.
Zlotta et al, J. Urol., 157:1315-1321, 1997.

TABLE 1
Markers for CRC used to evaluate tissue samples

| Gene | Marker for: | Reference |
|---|---|---|
| CGM2 | normal colon; down | Down-regulation of carcinoembryonic |
| | regulated in colon | antigen family member 2 expression is an |
| | adenocarcinoma | early event in colorectal tumorigenesis |
| | | Cancer Res. 57 (9), 1776-1784 (1997). |
| cMYC | early tumor | Identification of c-MYC as a target |
| | | of the APC pathway Science 1998 Sep 4; |
| | | 281(5382): 1509-12 |
| Cyclin | early tumor | The APC tumor suppressor controls |
| D1 | | entry into S-phase through its ability |
| | | to regulate the cyclinD/RB pathway. |
| | | Gastroenterology 2002 Sep; 123(3): |
| | | 751-63 |
| CEA | metastasis, recurrent | Differences in messenger RNA expression |
| | tumor | of carcinoembryonic antigen in surgical |
| | | specimens of colorectal carcinoma. |
| | | Tumour Biol. 1992; 13(5-6): 330-7 |
| GCCr | detection of colon | Guanylyl cyclase C is a selective marker |
| | cancer | for metastatic colorectal tumors in human |
| | | extraintestinal tissues. Proc Nat'l Acad. |
| | | Sci. USA 1996 Dec 10; 93(25): 14827-32 |
| EBAF | detection of colon | Distinct tumor specific expression of |
| | cancer | TGFB4 (ebaf)*, a novel human gene of |
| | | the TGF-beta super-family Front Biosci. |
| | | 1997 Jul 15; 2: a18-25 |
| PRL3 | metastasis | A phosphatase associated with metastasis |
| | | of colorectal cancer. Science 2001 Nov |
| | | 9; 294(5545): 1343-6 |

TABLE 2
Expression Values for DATAS ™ fragments.

| | | GenBank | | Score | Score | total |
|---|---|---|---|---|---|---|
| SEQ ID NO. | NAME | Accession | Event type | Early | Late | score |
| SEQ ID NO. 1 | EXH-DCTB0155-01 | NM_000609 | DATAS ™ | 1.7 | 0.8 | 1.15 |
| | | | fragment, | | | |
| | | | extension | | | |
| SEQ ID NO. 2 | EXH-DCTB1031-01 | NM_000018 | DATAS ™ | 0.2 | 0.6 | 0.8 |
| | | | fragment, | | | |
| | | | extension | | | |
| SEQ ID NO. 3 | EXH-DCTB1047-01 | NM_000018 | DATAS ™ | 0.0 | 0.7 | 0.7 |
| | | | fragment, | | | |
| | | | Novel | | | |
| SEQ ID NO. 4 | EXH-DCTB1107-01 | NM_015449 | Novel | 4.2 | 0.7 | 4.9 |
| SEQ ID NO. 5 | EXH-DCTB1704-01 | NM_024298 | Novel | 4.4 | 0.8 | 4.12 |
| SEQ ID NO. 6 | EXH-DCTB1734-01 | XM_300663 | Novel | 4.4 | 1.7 | 5.11 |
| SEQ ID NO. 7 | EXH-DCTB1839-01 | NM_000196 | Extension | 0.6 | 0.7 | 0.13 |
| SEQ ID NO. 8 | EXH-DCTB2178-01 | NM_005482 | Novel | 1.0 | 6.0 | 7.0 |
| SEQ ID NO. 9 | EXH-DCTB2695-01 | NM_001132 | Novel | 4.4 | 1.6 | 5.10 |
| SEQ ID NO. 10 | EXH-DCTC0602-01 | XM_301613 | Novel | 2.5 | 0.8 | 2.13 |
| SEQ ID NO. 11 | EXH-DCTC0638-01 | NM_024407 | Novel | 3.4 | 0.6 | 3.10 |
| SEQ ID NO. 12 | EXH-DCTC0907-01 | NM_004448 | Novel | 4.4 | 0.6 | 4.10 |
| SEQ ID NO. 13 | EXH-DCTC1082-01 | NM_006707 | Extension | 2.6 | 0.8 | 2.14 |
| SEQ ID NO. 14 | EXH-DCTC1227-01 | NM_025125 | Novel | 7.0 | 0.5 | 7.5 |
| SEQ ID NO. 15 | EXH-DCTC1257-01 | NM_005532 | Novel | 7.0 | 7.0 | 14.0 |
| SEQ ID NO. 16 | EXH-DCTC1743-01 | NM_030652 | Novel | 3.4 | 0.8 | 3.12 |
| SEQ ID NO. 17 | EXH-DCTC2089-01 | NM_032364 | Extension | 3.3 | 0.7 | 3.10 |
| SEQ ID NO. 18 | EXH-DCTC2107-01 | NM_015940 | Extension | 4.3 | 0.6 | 4.9 |
| SEQ ID NO. 19 | EXH-DCTC2167-01 | NM_005514 | Extension | 2.4 | 0.7 | 2.11 |
| SEQ ID NO. 20 | EXH-DCTD0627-01 | NM_004360 | Extension | 3.5 | 0.7 | 3.12 |
| SEQ ID NO. 21 | EXH-DCTD0660-01 | NM_004360 | Novel | 7.1 | 0.3 | 7.4 |
| SEQ ID NO. 22 | EXH-DCTD0784-01 | NM_003171 | Extension | 1.5 | 0.7 | 1.12 |
| SEQ ID NO. 23 | EXH-DCTD0841-01 | NM_006995 | Novel | 6.0 | 3.3 | 9.3 |
| SEQ ID NO. 24 | EXH-DCTD0880-01 | NM_021910 | Novel | 0.4 | 0.8 | 0.12 |
| SEQ ID NO. 25 | EXH-DCTD0991-01 | NM_004990 | Extension | 5.1 | 0.7 | 5.8 |
| SEQ ID NO. 26 | EXH-DCTD1192-01 | NM_018085 | Extension | 2.3 | 0.7 | 2.10 |
| SEQ ID NO. 27 | EXH-DCTD1519-01 | NM_145169 | Extension | 5.1 | 0.8 | 5.9 |
| SEQ ID NO. 28 | EXH-DCTD1536-01 | NM_004121 | Novel | 8.0 | 3.3 | 11.3 |
| SEQ ID NO. 29 | EXH-DCTD1650-01 | NM_002414 | Extension | 0.7 | 0.0 | 0.7 |
| SEQ ID NO. 30 | EXH-DCTD1786-01 | NM_002089 | Extension | 6.0 | 4.1 | 10.1 |
| SEQ ID NO. 31 | EXH-DCTD1930-01 | XM_049733 | Novel | 2.6 | 0.8 | 2.14 |
| SEQ ID NO. 32 | EXH-DCTD2188-01 | NM_019074 | Extension | 6.1 | 0.1 | 6.2 |
| SEQ ID NO. 33 | EXH-DCTD2280-01 | XM_305835 | Novel | 2.6 | 0.3 | 2.9 |
| SEQ ID NO. 34 | EXH-DCTD2285-01 | NM_023068 | Extension | 1.7 | 0.1 | 1.8 |
| SEQ ID NO. 35 | EXH-DCTE0390-01 | NM_014610 | Extension | 6.0 | 2.0 | 8.0 |
| SEQ ID NO. 36 | EXH-DCTE0424-01 | NM_032806 | Extension | 3.0 | 0.6 | 3.6 |
| SEQ ID NO. 37 | EXH-DCTE0536-01 | NM_138373 | Novel | 5.1 | 7.0 | 12.1 |
| SEQ ID NO. 38 | EXH-DCTE0648-01 | NM_001712 | Novel | 1.7 | 0.8 | 1.15 |
| SEQ ID NO. 39 | EXH-DCTE1361-01 | NM_014556 | Novel | 1.6 | 0.7 | 1.13 |
| SEQ ID NO. 40 | EXH-DCTE1434-01 | NM_005588 | Novel | 6.1 | 1.5 | 7.6 |
| SEQ ID NO. 41 | EXH-DCTE1480-01 | NM_005588 | Extension | 2.6 | 0.7 | 2.13 |
| SEQ ID NO. 42 | EXH-DCTE1956-01 | XM_290535 | Extension | 0.4 | 0.7 | 0.11 |
| SEQ ID NO. 43 | EXH-DCTE2152-01 | NM_000958 | Extension | 0.6 | 0.6 | 0.12 |
| SEQ ID NO. 44 | EXH-DCTE2366-01 | NM_000114 | Extension | 1.7 | 0.8 | 1.15 |
| SEQ ID NO. 74 | EXH-DCDA0001-01 | NM_001062 | Amplicon | 6.0 | 8.0 | 14.0 |
| SEQ ID NO. 79 | EXH-DCDA0015-01 | NM_033049 | Amplicon | 3.0 | 0.5 | 3.5 |
| SEQ ID NO. 85 | EXH-DCDA0020-01 | NM_000358 | Amplicon | 5.3 | 8.0 | 13.3 |
| SEQ ID NO. 91 | EXH-DCDA0037-01 | NM_002632 | Amplicon | 6.0 | 6.0 | 12.0 |

1. An isolated nucleic acid that is expressed by a human cancer cell, selected from the group consisting of:
i) nucleic acids comprising a sequence contained in SEQ NOS. 1-44, 47, 52, 53, 56, 57, 62, 65, 70, 73, 74, 76, 79, 82, 85, 88, 91, and 96;
ii) a nucleic acid having a sequence that is at least 70% identical to the sequence of (i) when aligned without allowing for gaps;
iii) nucleic acids having a sequence complementary to i) or ii); and
iv) fragments of i), ii) or iii) having a size of at least 20 nucleotides in length.

2. A nucleic acid of claim 1, consisting of a sequence contained in SEQ ID NOS. 1-44, 47, 52, 53, 56, 57, 62, 65, 70, 73, 74, 76, 79, 82, 85, 88, 91 or 96, or a sequence complementary thereto.

3. A primer mixture that comprises primers that result in the specific amplification of one of the nucleic acids of claim 1.

4. A polypeptide expressed by a human cancer cell, that is selected from the group consisting of:
i) the antigen encoded by a nucleic acid sequence having at least 90% sequence identity in SEQ ID NOS. 1-44, 47, 52, 53, 56, 57, 62, 65, 70, 73, 74, 76, 79, 82, 85, 88, 91, and 96, or a sequence complementary thereto,
ii. a polypeptide comprising an amino acid sequence having at least 90% sequence identity in SEQ ID NOS. 48, 49, 54, 55, 58, 59, 66, 67, 71, 72, 75, 77, 78, 83, 84, 89, 90, 97, 98 and 99, and
iii. an antigenic fragment of (i) or (ii).

5. A tumor antigen, comprising an amino acid sequence selected from the group consisting of SEQ ID NOS. 48, 49, 54, 55, 58, 59, 66, 67, 71, 72, 75, 77, 78, 83, 84, 89, 90, 97, 98 and 99 or an antigenic fragment thereof.

6. A method of detecting and/or staging cancer, comprising determining whether a human cell sample, particularly a human colon cell sample, expresses a target nucleic acid molecule, wherein said target nucleic acid molecule comprises the sequence of a gene or RNA comprising a nucleic acid sequence selected from the group consisting of SEQ ID NOS. 1-44, 47, 52, 53, 56, 57, 62, 65, 70, 73, 74, 76, 79, 82, 85, 88, 91, and 96; a sequence complementary thereto, or of a fragment of said gene or RNA having a size of at least 20 nucleotides in length.

7. The method of claim 6, wherein said method comprises detecting the expression of said target nucleic acid molecule using a nucleic acid sequence that specifically hybridizes thereto.

8. The method of claim 6, wherein said method comprises detecting the expression of said target nucleic acid molecule using oligonucleotides that result in the amplification and/or the detection thereof.

9. The method of claim 6, wherein the expression of said target nucleic acid molecule is detected by assaying for the antigen encoded by said nucleic acid.

10. The method of claim 9, wherein said assay involves the use of an antibody or a fragment thereof that specifically binds to said antigen.

11. The method of claim 10, wherein said assay comprises an ELISA or competitive binding assay.

12. The method of claim 10, wherein said antigen is a polypeptide as defined in claim 4 or 5.

13. The method of claim 6, further comprising comparing the expression level of said target molecule in said cell sample to a reference expression level, wherein a deviation from said reference expression level is indicative of the presence and/or stage of said cancer in said subject.

14. The method of claim 13, wherein said reference expression level is an expression level as determined in a control sample or a median expression level from healthy subjects.

15. An antibody or antigen-binding fragment thereof that specifically binds to a target polypeptide molecule selected from:
i. a polypeptide encoded by a nucleic acid molecule comprising the sequence of a gene or RNA comprising a sequence selected from the group consisting of SEQ ID NOS. 1-44, 47, 52, 53, 56, 57, 62, 65, 70, 73, 74, 76, 79, 82, 85, 88, 91, and 96; a sequence complementary thereto, or by a fragment of said gene or RNA having a size of at least 20 nucleotides in length,
ii. a polypeptide comprising the sequence of a protein comprising a sequence selected from the group consisting of SEQ ID NOS. 48, 49, 54, 55, 58, 59, 66, 67, 71, 72, 75, 77, 78, 83, 84, 89, 90, 97, 98 and 99; or a fragment of said protein having a size of at least 5 amino acids in length.
iii. an antigen according to claim 4 or 5, and
v. an antigenic fragment of (i), (ii), or (iii).

16. The antibody of claim 15, which is a monoclonal antibody or an antigen-binding fragment thereof.

17. The antigen of claim 4 which is attached directly or indirectly to a detectable label.

18. The antibody of claim 15 which is attached directly or indirectly to a detectable label.

19. A diagnostic kit for detection and/or staging of cancer, which comprises a DNA according to claim 1 and a detectable label.

20. A diagnostic kit for detection and/or staging of cancer, which comprises primers according to claim 3 and a diagnostically acceptable carrier.

21. A diagnostic kit for detection and/or staging of cancer, which comprises a monoclonal antibody according to claim 15 and a detectable label.

22. A diagnostic kit in the form of a sandwich ELISA in which at least one of the capture of the detection antibodies comprises a monoclonal antibody according to claim 16.

23. A method for detecting and/or staging cancer using human fluid, in particular whole blood, serum or plasma, as a sample source, with a diagnostic kit described in claim 19.

24. A method for treating cancer comprises administering to a human subject in need thereof a therapeutically effective amount of a ligand which specifically binds a target molecule selected from
i. a gene or RNA comprising a sequence selected from the group consisting of SEQ ID NOS. 1-44, 47, 52, 53, 56, 57, 62, 65, 70, 73, 74, 76, 79, 82, 85, 88, 91, and 96; a sequence complementary thereto, a variant thereof or a fragment of said gene or RNA having a size of at least 20 nucleotides in length, and
ii. a protein or polypeptide encoded by a gene or RNA comprising a sequence selected from the group consisting of SEQ ID NOS. 1-44, 47, 52, 53, 56, 57, 62, 65, 70, 73, 74, 76, 79, 82, 85, 88, 91, and 96; a sequence complementary thereto, a variant thereof or a fragment of said gene or RNA having a size of at least 20 nucleotides in length; or
iii. A protein or polypeptide comprising a sequence selected from the group consisting of SEQ ID NOS. 48, 49, 54, 55, 58, 59, 66, 67, 71, 72, 75, 78, 83, 84, 89, 90, 97, 98 and 99; a variant thereof or a fragment of said protein having a size of at least 5 amino acids in length.

25. The method of claim 24, wherein the ligand is a ribozyme or antisense oligonucleotide that inhibits the expression of a gene comprising a DNA sequence selected from the group consisting of SEQ ID NOS. 1-44, 47, 52, 53, 56, 57, 62, 65, 70, 73, 74, 76, 79, 82, 85, 88, 91, and 96, a sequence complementary thereto and a fragment or variant thereof.

26. The method of claim 24, wherein the ligand is directly or indirectly attached to an effector moiety.

27. The method of claim 26, wherein said effector moiety is a therapeutic radiolabel, enzyme, cytotoxin, growth factor, or drug.

28. A method for treating cancer, particularly colon cancer, comprising administering to a subject in need thereof a therapeutically effective amount of an antigen according to claim 4, and optionally an adjuvant that elicits a humoral or cytotoxic T-lymphocyte response to said antigen.

29. A method for treating cancer, particularly colon cancer, comprising administering to a subject in need thereof a therapeutically effective amount of a ligand which specifically binds to a protein encoded by a gene or RNA comprising a sequence selected from the group consisting of SEQ ID NOS. 1-44, 47, 52, 53, 56, 57, 62, 65, 70, 73, 74, 76, 79, 82, 85, 88, 91, and 96; a sequence complementary thereto or a fragment, or variant there, or a protein sequence selected from the group consisting of SEQ ID NOS. 48, 49, 54, 55, 58, 59, 66, 67, 71, 72, 75, 77, 78, 83, 84, 89, 90, 97, 98 and 99; optionally directly or indirectly attached to a therapeutic effector moiety.

30. The method of claim 29, wherein said effector moiety is a radiolabel, enzyme, cytotoxin, growth factor, or drug.

31. The method of claim 30 wherein the radiolabel is yttrium or indium.

32. The method of claim 29 wherein said ligand is a monoclonal antibody or fragment thereof.

33. The method of claim 29 wherein said ligand is a small molecule.

34. The method of claim 29 wherein said ligand is a peptide.

35. The method of claim 29, wherein said ligand binds an extracellular domain of said protein.

36. A molecule, selected from:
i. a polypeptide comprising the sequence of an extra-cellular domain of a protein encoded by a gene or RNA comprising a sequence selected from the group consisting of SEQ ID NOS. 1-44, 47, 52, 53, 56, 57, 62, 65, 70, 73, 74, 76, 79, 82, 85, 88, 91, and 96, or a sequence complementary thereto; and
ii. a polypeptide comprising the sequence of an extra-cellular domain of a protein sequence selected from the group consisting of SEQ ID NOS. 48, 49, 54, 55, 58, 59, 66, 67, 71, 72, 75, 77, 78, 83, 84, 89, 90, 97, 98 and 99; and
iii. a nucleic acid molecule encoding a polypeptide of (i).

37. The molecule of claim 36, wherein said polypeptide has 8 to 100 amino acids in length.

38. A method for selecting, identifying, screening, characterizing or optimizing biologically active compounds, comprising contacting a candidate compound with a target molecule and determining whether the candidate compound binds said target molecule, wherein said target molecule is selected from
i. a nucleic acid molecule comprising the sequence of a gene or RNA comprising a nucleic acid sequence selected from the group consisting of SEQ ID NOS. 1-44, 47, 52, 53, 56, 57, 62, 65, 70, 73, 74, 76, 79, 82, 85, 88, 91, and 96 or a sequence complementary thereto;
ii. a fragment of said gene or RNA having a size of at least 20 nucleotides in length, and
iii. a polypeptide encoded by (i) or (ii) and (iv) a amino acid molecule comprising the sequence selected from the group consisting of SEQ ID NOS. 48, 49, 54, 55, 58, 59, 66, 67, 71, 72, 75, 77, 78, 83, 84, 89, 90, 97, 98, and 99.


SEQUENCE LISTING (99 sequences)

SEQ ID NO: 1 - DNA, 190 bp, Homo sapiens
gatttttacccgaagctaaagtggattcaggagtacctggagaaagctttaaacaagtaagcacaacagccaaaaaggactttccgctagacccactcgaggaaaactaaaaccttgtgagagatgaaagggcaaagacgtgggggagggggccttaaccatgaggaccaggtgtgtgtgtgggggttcc

SEQ ID NO: 2 - DNA, 311 bp, Homo sapiens
tcgggaggggaggaatgacgaccactagactattagaggctacagccaccacttcccctcaggccctgagggctaaggagacctggctatgtcctattttgtgttggacttggttgctggagccaaaactttagctcagttgcatacatattagtcccaaaggccacattcagtccacctgctttggcttttgcacacgtccccagggtgacttgcaacagccatctgcccaggagcccagtcctgcccctcagtcctaagctcccccaacccaaattcactcactgggtaatgcccccgaagcccctgtc

SEQ ID NO: 3 - DNA, 224 bp, Homo sapiens
attagaggctacagccaccacttcccctcaggccctgagggctaaggaggcctggctatgtcctattttgtgttggacttggttgctggagccaaaactttagctcagttgcatacatattagtcccaaaggccacattcagtccacctgctttggcttttgcacacgtccccagggtgacttgcaacagccatctgcccaggagcccagtcctgcccctgcca

SEQ ID NO: 4 - DNA, 242 bp, Homo sapiens
gggttgcaggtagggaggccatggcgtccggcagtaactggctctccggggtgaatgtcgtgctggtgatggcctacgggagcctggtgtttgtactgctatttatttttgtgaagaggcaaatcatgcgctttgcaatgaaatctcgaaggggacctcatgtccctgtgggacacaatgcccccaaggacttgaaagagcagattgatattccactctccagggttcaggatatcaagtat

SEQ ID NO: 5 - DNA, 401 bp, Homo sapiens
ggtcgagagggatgataaccacaacttccctggcaactgcagtgtccgacaatttagaagggaccatccttggcggcttctctgaatatactgagtttggttgctaaaggactcatagcttagcaaccatagcccttcaaggcttttcatggctgtggcgggccccattaggtaccaaaagaagaagaaccccattgtcagtgaactgtaccacccagcccacccaccttcctaccctacaggcaccctctgggccaccctcccttgctgccctagcaagtctgacagccagagggccattgcctggccaggatcccttccttagcatccggggctgggacactagcaggcgtcgggagggggcctggctgagctgcatgtctgtcccccaccctcaccaa

SEQ ID NO: 6 - DNA, 371 bp, Homo sapiens
ggattggagggccttgttttcccagtaatccacccaccgccacttcaagaagaaatgatatgaagaagtgccggttctccctcccctcttccgcactgtcccgtgatgatgacgcctccagagaggacgataatctgggttcctgggagagatggcttggtcactattcccacccttgcctcgaccacttgtctcaatgtcaccacctcacgccctgttccaggtggctgagtccgaatccagtaaccaccacctcgttttggttaatctcaggctcgggtgttgtagcaacattggaaatgggaggggtttacgaagtgaacatgaggtcaggtgcctgaattcaacagtctacccattccccttgccaa

SEQ ID NO: 7 - DNA, 273 bp, Homo sapiens
ggtcatggttttggttgggggtgtagttttggctgcagacctggcgcgggttaaacagtcctaattggctttggctcccctagctgatcccactctgaccttgccactccttccccagagtcagtgagaaacgtgggtcagtgggaaaagcgcaagcaattgctgctggccaacctgcctcaagagctgctgcaggcctacggcaaggactacatcgagcttgcatgggcagttcctgcactcgctacgcctggccatgtccgacctaccatc

SEQ ID NO: 8 - DNA, 312 bp, Homo sapiens
gattttgacccggagaaccccacgaaatcatgcaaatcaagaggttccaatcttcgtgttcactttaagaacactcgtgaaactgctcaggccatcaagggtatgcatatacgaaaagccacgaagtatctgaaagatgtcactttacagaaacagtgtgtaccattccgacgttacaatggtggagttggcaggtgtgcgcaggccaagcaatggggctggacacaaggtcggtggcccaaaaagagtgctgaatttttgctgcacatgcttaaaaacgcagagagtaatgctgaacttaagggtcaacct

SEQ ID NO: 9 - DNA, 499 bp, Homo sapiens
  modified_base (426)..(426): a, c, t, g, unknown or other
ggcatgcaagcttgagtattctatagtgtcacctatataccaggcggatgcagaaggggggacttcccctgggatgacgaggatttccgcagtctggcccttttgggggcaggcgttgccatgggatttttctacctctattttcgagatcctggaagagaaatcacgtggaagcactttgtacagtattacctggccagagatctggtggaccggctggaagtcgtgaacaaacaatctgtgcgtgttattcctgcccctgggacctcttctgaaacgtagctgggtgcgaagaatcaaattggaaattatggaatttgtgaatttcctgaagaatcccaagcagtatcaggacttaggagcaaaaattccaaagggagccatgctcactggtcctcctggtaccggcaagacccttcttgccanagcaactgcaggggaggccactgcgcccttcatcactgtgaacgggtctgagttcctggaaatgtttattggc

SEQ ID NO: 10 - DNA, 492 bp, Homo sapiens
tacgtgcaggggccatcatcggaagggcctgaggaggaggacggagaaggcttctccttcaaatacagccccgggaagctgaggggaaaccagtacaagaagatgatgaccaaagaggagctggaggaggagcagagaactgaagaataacgaagttatccttagcgtcctcctaaaggcttttccttttggcatcttaaaagcttgagagataaaacggaaaccccagagaggagtctgggcaggctcccagggtgcatgctgcctccataaatctgctgagctctagaccctcaatcaggacttgtcccttggctagcaggatcctgggaacacctttggccctgccctgtgtagagatgttcatgtctgttcctgtgggtcactttgttaagctgaagagttttaagaggtagagctcagaccctggactgggatttttcttaccactcaaacttgctatccacacacccttcattcaataacgcttct

SEQ ID NO: 11 - DNA, 458 bp, Homo sapiens
tgaaggcagggaggagcacggtccccaaggagggaggagcgcagtccccagacagggagcacagtccgcagggagggaggagcgcggtccccagggagggaggagctcagtctgcaaggagggaggagtacagtccacactcgcaacttgccccattggtctgcactgcaggggaagacgccaggggcatgggcagagctcctgaccccaggatggacctctctgtgctgtcaagtcacagggaggcccaggctgccctctccactgcccccggggtgacctgaaccgtgcagaacgctgaacaaatcaacgccctttccagggaagcggaatccaaagtcagagcctgttcctccacttttgagaggcaccaggatgtgcctctcgcttgcccaaacccccagactcgggactcagggctgggctctctgcgcatgagctaatgccgcacgcagcac

SEQ ID NO: 12 - DNA, 453 bp, Homo sapiens
gatgaaggggggcagcgagggggattgccagggacttggcaggatggcgagatgcagtagggtgtgctatctggtaaaatatccctggagagggctcagcgctcagacctgaacagcaacagagtggcagaaaaggggcctgggggacactggggcccttcagactatgaaaaggttctaaggaggtctgtgttggtggctgtgactgtggctgtgctagggtggtgagccctgtgggctcaggcgtcagactacctggattcagacccagctcctgcttccaactttggttttttattcctaaaatgggtattgtaataatacctaccttgctggggtgtggcaagaatgaaattaaacagggcttggcacagtgaagcacgggaaaggctttctacagagcagtgactgttgttactcgctgttacaccttaggtaatgcgttttcctctc

SEQ ID NO: 13 - DNA, 482 bp, Homo sapiens
tgggcaggctgataaaatggggattgaatgtgaaatacaaatgttctgttgtcagtctgaggacccaatacccattgttgggagacaaagtcacattgttcttccccctgtctacgtcatcccgacacactcccacataccaccctacattttgtcccacgtccacctcccagtaatgtttccctgcttggaaaccctgagaagccaccacactcttccttgtaaatctcttctcagagtgaggcacctcctggggagcttttctatgggttacagttttcagatcagaaacgcagagcttcgggtgagccatctctggatccagagtcacctccactggggtgcggtgggggagagaggaagcatgagttactgaagacgaaatctgggctggagcaagaggtcgaccctcccactgcagactggttccgggtaggatccggccagtcccactatcccacagccatgtaccttctccaggg

SEQ ID NO: 14 - DNA, 124 bp, Homo sapiens
gggagagtccttgtatgccatagtattgtgcaagcaaaatgagctaagaatacggataaaggtatgtgaatgagaacaagttatgatgaccccaggaaaacccttctccgggttacctgcataa

SEQ ID NO: 15 - DNA, 483 bp, Homo sapiens
gcggccgaggtcagcttcacattctcaggaactctccttctttgggtctggctgaagttgaggatctcttactctctaggccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggccatggcggctgtgcccatggtgctcagtgccatgggcttcactgcggcgggaatcgcctcgtcctccatagcagccaagatgatgtccgtggcggccattgccaatgggggtggagttgcctcgggcagccttgtggctactctgcagtcactgggagcaactggactctccggattgaccaagttcatcctgggctccattgggtctgccattgcggctgtcattgcgaggttctactagctccctgcacc

SEQ ID NO: 16 - DNA, 447 bp, Homo sapiens
gggcaaggcggaaaaagatgagcgcgctctgaagcaggagattcacgagctgcgagggcgcctggagcggctggacaggtgagccaagcctgctgggtggggcgaggccagacgtcactgtcaataccctgaggcatctcttcctttctagtgggccggtcaggctggggcctgggtcagagcggtgctgcccgtgccgcctgaagagctgcagccagaacaggtggctgagctgtggggccggggtgaccggatcgaatctctcagcgaccaggtgctgctgctggaggagaggctaggtgcctgctcctgtgaggacaacagcctgggcctcggcgtcaatcatcgataagaagcctctacagcacccctgccccctaatttatacagaaaccggacccgctaatcctctgggattggccgactgtgagctgcagataaggctat

SEQ ID NO: 17 - DNA, 255 bp, Homo sapiens
tgaagagggggccttcaaggttttgcgagcagcttgggacattgtcagcaatgctgaaaagcgaaaggagtatgagatgtaagttggagatgggaagtcatcagataatggtaaatgaaaaatcctcaatagcagaggcatctggacttgggggtggaggcttgttgagatggagagaactgaagtcacttgtctttctcgctagacaggggcctcaagaggccaactgatatgtcttcctttgtccctacactt

SEQ ID NO: 18 - DNA, 312 bp, Homo sapiens
tggttgcagggggaaatggagctgaccctgttgctgtaagtttctgacaatttctccagtactttctttggacctgcttcgagcaacaggattttcttgtcatgaaagtgaatatcatatccttaaaaaaaaaaaatttatatcatcttaggaaaatcaagaatttcctttcccattaccaagagaaacaacagagtaaccacttaacccttaaaatatgaatttcttacagagaacataaacactatacccatgcagagtcctaagcgataacatataccaccattttagctatgagaaccaccctccaca

SEQ ID NO: 19 - DNA, 389 bp, Homo sapiens
  modified_base (274)..(274): a, c, t, g, unknown or other
tatggtgaggggaaggtccctgctaaggacagaccttaggagagcagttggtccaggacccacacttgctttcctcgtgtttcctgatcctgccctgggtctgtagtcatacttctggaaattccttttgggtccaagacgaggaggttcctctaagatctcatggccctgcttcctcccagtcccctcacaggacattttcttcccacaggtggaaaaggagggagctactctcaggctgcgtgtaagtggtgggggtgggagtgtggagganctcacccaccccataattcctcctgtcccacgtctcctgcgggctctgaccaggtcctgtttttgttctactccaggcagcgacagtgcccaaggctctgatgtgtctctcacag

SEQ ID NO: 20 - DNA, 374 bp, Homo sapiens
gggatgaacccaggaggcggaggctgcagtgagccgagattgtaccattgcaccccagtgcgcccattgcctggcctctccttgtcacatcttctccttgaagcttgctttcagttagccaggtgtggtggtgcatgcctgtggtcccagctactctggaggctgaggtgggaggattgcttgagcccaggaggttgaggctacagtgagccatgatcgctcaaatacactccagcctggtgacagtgagatcttatctcaaaagaacaacaaaaaaagaggaatcctttagctccctgagactcagctctgctagcagtcttggtactttgtaaatgacacatctctttgctctgcagtacaagggtcacccc

SEQ ID NO: 21 - DNA, 271 bp, Homo sapiens
gggatgaacccaggaggcggaggctgcagtgagccgagattgtaccattgcaccccagtgcgcccattgcctggcctctccttgtcacatcttctccttgaagcttgctttcagttagccaggtgtggtggtgcatgcctgtggtcccagctactctggaggctgaggtgggaggattgcttgagcccaggaggttgaggctacagagagccatgatcgctcaaatacactccagtctggtgacagtgagatcttatctcataagaacaac

SEQ ID NO: 22 - DNA, 197 bp, Homo sapiens
tgagtggaggcagaggtcgcagtgagccgagatagtaccattgcattccagcctggacaacagagcaagactctgtcttgaaaaaaaaaaaattaaaaataccaaaccatagtttcttaaacagcatatacttacaaggagttgtaacactgcccatcccaactgtacaaaaaaaatgtgaagcctgtttcccattt

SEQ ID NO: 23 - DNA, 443 bp, Homo sapiens
aatagggaccatagggaactggggtcagttcatcaacggtgtcccagcaatgatgcatcatggctcttctgtcagggaaccccagcagctcttaatgaaccctgcagtttacatccccaggaccctttctcttttggaataatttaatgcatgattatcaaaggtttcccatgcatagtagcctcctaagagttacatttcagaagtcattgctccttgattctggatcaatttgaaataagataatgctgtcttagcaattcatatcctgggaatggtatagactgtgcacagttgaagacctatgtgaggaaaaggccataagggtccccagtaagcctgatgcaatggtgtacaaataggtggccagaaatacctttctgcctctaagagagcactgatattctgcatgtagtctgccaggggaaggggtttgggtgagt

SEQ ID NO: 24 - DNA, 334 bp, Homo sapiens
tcagggaagggggtatagtggggccttgcaggccagaggtggcttggaggagcccctggaaagaggcttaagaggtgagactcaacagccatggcgacagagcatagggctttaagatgaatttgcaggggttacaggattacaactgcaatgtgggctaataatagtgccccctgcattaagctgcagagattgagcgagtaagtgggaagctgagaaaatgcccccatggggtagacactcaataagcatctgctgttattaccaggactcgtatggtcatgggtgacagcttcagaccacaggcagtccacctacaacctgtgccccttgc

SEQ ID NO: 25 - DNA, 321 bp, Homo sapiens
tatgggcaggtaacagtgaggcaagaaaaagatctatttaggattcagcttgtccagtctcccacagggcttaagcttcatacttgttttgtcacttcatccatcagcgcttgtatctgctgtggcttggctgttgtaacagtctctacaactgctggcttcggggacgtttttgcctggagaacaacaaagttatcaccaacaaccataaatatcccctaacctccagttttatacagcatctcagagggaaagtggttacccttaagtcgaaggtctcttctagttaagacaggaaagaaaaactgtaagtgaggaagc

SEQ ID NO: 26 - DNA, 239 bp, Homo sapiens
ttgtcaggcagggggattccgtgttgccttatccacttcagatcccgtcgtcgcctcactggctcaggacatcttcaaggagctgtcccagattgaagcctgtcagggcccaatgcaaatgaggctgattcccactctggtcagcataatgcaggccccagcagacaagattcctgcagggctttgtgcgacagccattgatatcctgacaacagtagtacgaaatacaaagcctcacc

SEQ ID NO: 27 - DNA, 478 bp, Homo sapiens
ggtggaggcaacagagtggcggccgctacggccctgtaacagggccatggagaagctgcggcgagtcctgagcggccaggacgacgaggagcagggcctgactgcgcaggtcctggatgcctcatcccttagtttcaacaccagattgaaatggtttgccatctgcttcgtatgtggcgttttcttttctattcttggaactggattgctgtggcttccgggcggcataaagctttttgcagtgttttataccctcggcaatcttgctgcgttagccagtacatgctttttaatgggacctgtgaagcaactgaagaaaatgtttgaagcaacaagattgcttgcaataattgttatgcttttgtgtttcatatttaccctgtgtgctgctctttggtggcataagaagggactggccgtgttattctgcatattgcagttcttgtcaatgacctggtatagcctgcgccataacgct

SEQ ID NO: 28 - DNA, 337 bp, Homo sapiens
tggccaggctggtcttgaactcctgagctcaaaagatcctcctgcgccggcctcccaaagtgctgggattacaggtgtgagccaccacgcccagcccaatttttgattttcttttagagacggtgtcttgctttgttgccctggctgttctcaaactcctggcctcaagtgatcctgccaccttggctcccaagtggctaagactgtaggtatgtgccaccacgcctggcttttttttatttcttattttttattttttagaggcaggatcttgctgcattgcccaggctggtctcaaactcctggcctcaggtgatcctccttcctcggcctactc

SEQ ID NO: 29 - DNA, 402 bp, Homo sapiens
gggccaggcagccacaggaaagaaggggaagaggccgacgccccaggcgtgatccccgggattgtgggggctgtcgtggtcgccgtggctggagccatctctagcttcattgcttaccagaaaaagaagctatgcttcaaagaaaatgcagaacaaggggaggtggacatggagagccaccggaatgccaacgcagagccagctggtaagaaggacggggaacgatggcttgcacacgtggccagtgttcccattttatcttctccatcctctcccatcttgctgtcctgctcacattctcaaatttggttgcatggctttgaatgtcttcctttatgtctcgttgctttggagggatactttcaaaagacaatgaatgtgtaaacttcctagggcctacac

SEQ ID NO: 30 - DNA, 251 bp, Homo sapiens
ttgttagacccgcatcgcccatggttaagaaaatcatcgaaaagatgctgaaaaagtaagttataatttccatgtacacaggcgactggagctgttggtcagaaatactggcgtctgccccctaaaaagtaaatcaggaaaacccagggttagctgcaggactgaaaaaattattattttcacaaagttgccattaaggttattaatctgttctggtgccagaggatattcccagtgcccagggtatcccc

SEQ ID NO: 31 - DNA, 397 bp, Homo sapiens
tgttgttaccccggagatggaggttgcagtgagccaagatcgtgccattgcactccagcctgggcaacagggcaagattccgtctcaaaaacaaacactattagaaaatgccctggaggtggcggggagttgttgatttgtgaggacagattgaaagcaactcccagggtggccttgtccacctccccgtcgagaatgtggctgccggcctctttgaagattgtggtctggcataaggagaggtgcaggcgcctggttctgagcaccttggaatttccagccgcacagcatctggtgccctcccctccaccctcacaaggagctgccatcctgtttggattttctgtttgtggaccagaaacaaacgtttttccaaaggattagcaaatagggtacc

SEQ ID NO: 32 - DNA, 381 bp, Homo sapiens
tgtaggaagggcaggaagggcacaaggggtgaggggccccctcacccatcagaggctggccagcagtgccgtggctgcccaacccagcaccaatacagcaggaaaggaccagaccaggcttgccgccatggttcctgtttcacacccttagtggcaatgaggccagcagggacccaagagtccactgaacttggcctgccaaggcagggttctgacctacccttcaggaatggcccatgggtcaagagtgaccaggccactttccctcccacaaaaggcagaaggaaaggcgagcccagcactcacctccgtggcaatgacacattcattcctctcctctgatatcaaacacacagactggtacatggagtccctacacat

SEQ ID NO: 33 - DNA, 230 bp, Homo sapiens
gcctgccaggttggtctcaaactcatatcctcaagtgatccaccctcctcgtcctcccaaagtgctgggattttgccatgagccaccacgctcagccatgtttagccatttttaaaaggtgtgaacagataattaagtctattagcaacattagaaaatttaagttaaaatttaaatgactctagcaagaattagcctttaggtgccctgggttcccctcctcacctccc

SEQ ID NO: 34 - DNA, 452 bp, Homo sapiens
gccggggaggtggggaggggagaggctctctgagtccactccgggccttgcagaaggaggcgtgtttgtaagcagagcatgggcgagaattcggtggagatggcttttcagaaagagaccacgcaggtatccattcaacacacacgggcagatataatacccagctaacccacagtgccaactggcatctggagactgctgtcatttagaggatggtgcctgggaccaaggggaggttggggagagtgtgcagggaaccaggagcccctcaggagataggagagccctgttctgctcggggcagagggaatccctgtgtcagctggcggctggcttagaggactaggggagactttctggacttggcagcatcccctccatttccaagacacgtatgttctgactcccccacctttgttctgaggcctcttcccactcccagcctgcccgtc

SEQ ID NO: 35 - DNA, 462 bp, Homo sapiens
aagggtcgaactccaaggcagaagaaagaatatctctcagcactaatgctccccttccctccccctaacccagaacatcccttgggttatcgcaggtgaatactccaatcagatgccacattgatgccaggcttgcgcaggaccaacacagaggtctcagggtcatgctggaaggacaggcggctttctggagatccttttgtctggagtaccacagctgctggctttccagcccctattatcaccacccgctcaatccagattggtgtctcaaagtgtccttcagggtctgctgagctggagacaagggtgttgccagagaatgagaatcgacgcagcaggaactcttggcgagtctggtagttgaacgtgtgcccatcatccagaaagagctctccttgagctgtaccctgagggctaagtgcacaaagagagtgatggggtcatccttcatacattctg

SEQ ID NO: 36 - DNA, 467 bp, Homo sapiens
aggaggtggcttcgtcccgcggagtccaggcttcaggctttcaccagttctcaggatgcccatagggatgggtgaagcctgcctggcctgtggtgcttcccagtggccgtcatctcattagggccccacagtggcattaggatgcacctctcggcggtgttcaacgccctcctggtgtcggtgcaggcagcggtcctgtggaagcatgtgcggctgcgtgagcatgcagccacactggaggaggagctggccctcagccgacaggccacagagccagccccagcactgaggatcgactacccggaggcactgcagatcctgatggagggcggcacacacatggtgtgcacgggccgcacgcacacagaccgcatctgccgcttcaagtggctctgctactccaacgaggctgaggagttcatcttcttccatggcaacacctctgtcatgctgcccaacctctcacc

SEQ ID NO: 37 - DNA, 476 bp, Homo sapiens
ttgagaggcagtgtctggctccgggtggagcctcccaggtgctcttacagcctgttccaagtgtggcttaatccgtctccaccaccagatctttctccgtggattcctctgctaagaccgctgccatgccagtgacggtaacccgcaccaccatcacaaccaccacgacgtcatcttcgggcctggggtcccccatgattgtggggtcccctcgggccctgacacagcccctgggtctccttcgcctgctgcagctggtgtctacctgcgtggccttctcgctggtggctagcgtgggcgcctggacggggtccatgggcaactggtccatgttcacctggtgcttctgcttctccgtgaccctgatcatcctcatcgtggagctgtgcgggctccaggcccgcttccccctgtcttggcgcaacttccccatcaccttcgcctgctatgcggccctcttctgcctctcggcctca

SEQ ID NO: 38 - DNA, 381 bp, Homo sapiens
tgagggcacccaggaggtggagtttgcagtgagccaagattgcgccattgcgctccagcctgggcgacagagggaaactctgtctcaaaaaaaaaagaagttttcattttaccttttcaatagagcaaacatacatactgaaaagtgctaaatcataattatactttcacaatgtatgaactttcacaagttgaacacacccaggtaagcagcacccaaatcatgaaatactacatgacctccccttaaaaagagacccttccagccaggtgcagtggctcacacctgtaatcccagcactctgggaggccaaggcaggtggatcactagaagtcaggagttctaaaccagcctggccaacatagtgaaaccttgttctac

SEQ ID NO: 39 - DNA, 421 bp, Homo sapiens
gggattgaggggagagagaagaggatcagcagtgcagccccaggatgcctcgcagacgtgccttctgccatgattgtgaagttccagccacgtggaacttgtatctctagctcatggaaatggaaggaagaaatgtgaagtttaggagattggagcttccagtctgaggctgatccatacaatacaagaagaaagagagccaggaaaggcccaacaggagggaatcaatgcaagatttgagcctattttgtggaagcccgcccatcagccgtcttccctctcttcgcctccttgggtctcaggaactcagccttcagaagtcatgaactgaatctctcctcccacccccacatccaccagtctctaccaagagtgagtcctcaggaagcagccaccaccattcgttatcacgcggggggac

SEQ ID NO: 40 - DNA, 400 bp, Homo sapiens
gggaggttaaaacaatgtgttgtaggagaatcacattttattcaattggtcaaggactcacatctttattttaattgtccttgaaagaaactgaaattttagtgtcatctttttgataattttatcagaggtaaccgttcactgatttcaatttagtttccatcagcctatccatgctaattttatgcttcattttgcaaattcaataaataaaaatgtgactggattactttaaaataaaagtagctaatactgtgggaatcagctggattcaaagccacttttccatgtcactaggggtgatgggagatagaagcagcctggggaactttcaccagaagtgggagttaattgtagtcacttactaatcctggatcttcctcatgactctctatttcct

SEQ ID NO: 41 - DNA, 416 bp, Homo sapiens
gggaggttaaaacaatgtgttgtaggagaatcacattttattcaattggtcaaggactcacatctttattttaattgtccttgaaagaaactgaaattttagtgtcatctttttgataattttatcagaggtaaccgttcactgatttcaatttagtttccatcagcctatccatgctaattttatgcttcattttgcaaattcaataaataaaaatgtgactggattactttaaaataaaagtagctaatactgtgggaatcagctggattcaaagccacttttccatgtcactaggggtgatgggagatagaagcagcctggggaactttcaccagaagtgggagttaattgtagtcagttactaatcctggatcttcctcatgactctctatttcctgaagatatcacccacc

SEQ ID NO: 42 - DNA, 450 bp, Homo sapiens
ggggaggtggccaaagtcccaactgtgagccaggccccacattcactgggcctcctccagggtctgtatgccatggaaccctggacatggggctatgaaggaaggtgggtgttgctaagcccaggagcatgggcccctaaccttggccctgtgccccaggtgaggctggtgccaagttcattgaggtatctaaagaggcccggaagcggttcctgggccccctgcacccctccttcaacctggtaaagatcatccgcagtttcctgctgaaggtcctgcctgctgatagccatgagcatgccagtgggcgcctgggcatctccctgacccgcgtgtcagacggcgagaatgtcattatatcccacttcaactccaaggacgagctcatccaggccaatgtctgtagcggtttcatccccgtgtactgtgggctcatccctccctccctcc

SEQ ID NO: 43 - DNA, 299 bp, Homo sapiens
cttgttcaggctggtgggagcggcagggctgggcctgcccctaaggggagctccctgcaagtcacatttcccagtgaaacactgaacttatcagaaaaatgtatataataggcaaggaaagaaatacagtactgtttctggacccttataaaatcctgtgcaatagacacatacatgtcacatttagctgtgctcagaagggctatcatcatcctacaactcacattagagaacatcctggcttttgagcacttttcaaacaatcaagttgactcacgtgggtcctgaggcctataaac

SEQ ID NO: 44 - DNA, 213 bp, Homo sapiens
actgccgggcatgagctttggatggtggaggtctaaagcctgcttgctttgttggtccttgacttcaacctgtcttggggaaagggggcaaaagaaaaaaaagaaaggtatgttatgaaagtgtacctctgtagttctgtgaagctgaaaaagggaacgtcttcccactgcctctccagcctgttccctgattggaactgtatgtaccctatc

SEQ ID NO: 45 - DNA, 2040 bp, Homo sapiens
gaagcgcgctcccggggaggtgttgcagccatggctacggcagccggcgcgacctactttcagcgaggcagtctgttctggttcacagtcatcaccctcagctttggctactacacatgggttgtcttctggcctcagagtatcccttatcagaaccttgggcccctgggccccttcactcagtacttggtggaccaccatcacaccctcctgtgcaatgggtattggcttgcctggctgattcatgtgggagagtccttgtatgccatagtattgtgcaagcataaaggcatcacaagtggtcgggctcagctactctggttcctacagactttcttctttgggatagcgtctctcaccatcttgattgcttacaaacggaagcgccaaaaacaaacttgaagttgtctgaaagcttgctctacacttttacattcatcctcacccttttttttgtggggtagaggaggtgcagtaatttactcagtgatctttctactttctagaaactgtccttcaaagctctttaagaccccctcgttagtcagttttttctcttatatgctctggttgagcttgaatagaccagttgttacttaagaaagaaacagagaaagattttagcttttcaatcctatttggcagaggacttcagctaccttcttacagtctttggctgtgttggtaccctcgtgtgctctgagctaagccacatactaaactgactttttggtttgtatacccttgctcccgccttctgatgaaaacaccttaccctcacaaccaccatctttcctctcctttccaaagctctttccaccttgctgcactaagataaagtgacactttcactatatgtcaattccacacacatttattaggtacctgtgaggtaggatcctatcctctcaaacttccatttctcatgctacagagaaagataaggaagatgagcaagtgcctggaatggggcaggctgagcagtcacacaggcatagaggcacgctgagaacctggaggggagactgcagagtgccttccctgatgctgcagccggaagtgatccttccctccacctggcccctgggacactgtgctctgcagtgtgcagggcctgatggcactgctagattgctccttcagctcagggccacagcttaaacagctttacctttcccctcagcacctgtcccactatcttgcacacaggtgctctaaccatgtttattgaacaaaggagggaaactgatttcactttcacttgttcattatcattccaatttttatgtgaaaatggcacaacccatttggggtaccctcaccccaaaataaaagcccaagtctacctttgactggtaccaccttttttgtggtttcgttggtgagaaacctttatctttttcatacctttctattctcaatcacttctccaaaagtgtgtctttccagctctgatttattcaaaacacaagcatttctgtttagagattctagcccatgggttatctggctagttattacctctcctgttcacttagttatactttattattgctcacaggctggggaggcagaatgactctgtcaccactaggagccattagggcttcttccctggaggactgcctgcttgctttctggggacactagccctcatttcccttctgtggtacagtggggcaaattatttgtattaagcaaacatttatgggaaacaacccgctcccgaaaacggagcccccaagtaaagcacaaccctgaaagattatgaactatgaattgtctctagtagagataaatttctgcaaacatatctcagtcttccctctgtttctctggtgattaagaagttcctttttggtaaggaaaaggatttttaaccatagagttaggcatcatggaaattcaaaccagatttcttaatacctggtcttcctcaaagagaaataataacagtaatagtggtgctgggaacaatatggcagattattgaatgaaattgattaacttgaataaaatgctgtgaattttctctaaaaa

SEQ ID NO: 46 - protein, 123 aa, Homo sapiens
MATAAGATYFQRGSLFWFTVITLSFGYYTWVVFWPQSIPYQNLGPLGPFTQYLVDHHHTLLCNGYWLAWLIHVGESLYAIVLCKHKGITSGRAQLLWFLQTFFFGIASLTILIAYKRKRQKQT

SEQ ID NO: 47 - DNA, 291 bp, Homo sapiens
atggctacggcagccggcgcgacctactttcagcgaggcagtctgttctggttcacagtcatcaccctcagctttggctactacacatgggttgtcttctggcctcagagtatcccttatcagaaccttgggcccctgggccccttcactcagtacttggtggaccaccatcacaccctcctgtgcaatgggtattggcttgcctggctgattcatgtgggagagtccttgtatgccatagtattgtgcaagcaaaatgagctaagaatacggataaaggtatgtgaatga

SEQ ID NO: 48 - protein, 96 aa, Homo sapiens
MATAAGATYFQRGSLFWFTVITLSFGYYTWVVFWPQSIPYQNLGPLGPFTQYLVDHHHTLLCNGYWLAWLIHVGESLYAIVLCKQNELRIRIKVCE

SEQ ID NO: 49 - protein, 12 aa, Homo sapiens
QNELRIRIKVCE

SEQ ID NO: 50 - DNA, 2414 bp, Homo sapiens
ggggtgagggcagcagctcgccacagctgccagccatctgtccattcacccatctgtccatctggcagcccgctgttcagacctgtctgtctgtccgcccatctctgtaagcccatctctgtcccattgtctatctgaccatctttctcttactgtcctctttgtctagctatctggcctatctgtcgatccatcttcgtgtctgtcttcagcccccacctgtttttgtccatctgtccaattacctgtgactctgtgcatcttcttgtccattcatctgcccacccatccgtccctccgtctgcccaccagccgcccctctcctcctgggctgcagagccatggcccggggctacggggccacggtcagcctagtcctgctgggtctggggctggcgctggctgtcattgtgctggctgtggtcctctctcgacaccaggccccatgtggcccccaggcctttgcccacgctgctgttgccgccgactccaaggtctgctcggatattggacgagccatcctccagcagcagggctcacccgtggatgccaccatcgcggctctggtctgcaccagcgtcgtcaaccctcagagcatgggcctgggcggaggggtcatcttcaccatctacaatgtgacaacagggaaggtggaggtcatcaatgcccgggagacggtgccggccagccacgccccgagcctgctggaccagtgtgcacaggctctgccactgggcacaggggcccagtggatcggggtgcccggggagctccgtggctatgccgaggcccaccgccgccatggccgcctgccctgggcgcagctgttccagcccaccatcgcgctgctccgaggggggcatgtggtggcccctgtcctcagccgtttcctgcacaacagcatcctgcggccttccttgcaggcgtcaaccctgcgccagctcttcttcaacgggacagaacccctgaggcctcaggacccactcccatggcctgcactggccaccaccctggagaccgtggccacagagggcgtggaggtcttctacacggggaggctgggccagatgctggtggaggacattgccaaggaagggagccagctgacgctgcaggacctggccaagttccagcccgaggtggtggatgccctggaggtgcccctgggggactataccctgtactcaccaccgccgcctgcagggggtgccattctcagctttatcctcaacgtgctaagagggttcaacttctcaacagagtctatggccaggcctgaagggagggtgaacgtgtaccaccaccttgtagagacgctcaagtttgccagggggcagaggtggaggctgggggaccctcgaagccacccgaagctccagaatgcctcccgggacctgctgggggagaccctggcccagctcatccgccaacagatcgatggccggggggaccaccagctcagccactacagcttggccgaggcctggggccacgggacaggcacgtcccatgtgtctgtgctgggggaggatggcagcgccgtggctgccaccagcaccatcaacacaccctttggagcgatggtgtattcaccacggacaggcatcatcctcaacaacgagctcctggacttatgcgagcgatgcccctggggttccggcaccaccccctcacctgtgagtggagacagggtgggtggagctcccggaaggtgctggcccccagttccaggcgagcgttccccatcctccatggtgccctccatcttgatcaacaaagcccaggggtcgaagctagtgattggcggggctggcggggagctcatcatctctgctgtggcccaggccatcatgagcaagctgtggcttggctttgacctgagagcggccattgcagcccccatcctgcatgtcaacagcaagggctgtgtggagtacgagcccaacttcagccaggaggtgcagaggggactccaagaccgtggccagaaccagacccagaggcccttcttcctgaacgtggtccaggctgtgtcccaggagggggcctgtgtgtacgccgtctcggacctgaggaagagtggggaggccgcaggctactaagacactgctctgcccagagctgaagtctggccccaccatgagtcctgtgtccaggccggacatggctgggggaccaactactctggcaggatctggacccctggcaggggagtccagctgagagtggaagaggtggcggggaccagctgggcagatgagaggctgagcctcatccctaaccccctttcccagagcccctggtggtcctgaaccggcccctctatccctccgcaggcctcttacctggggccactctcccaccctctcgatctgtatatcctccagtccaagattaaagaagaggcggactgt

SEQ ID NO: 51 - protein, 586 aa, Homo sapiens
MARGYGATVSLVLLGLGLALAVIVLAVVLSRHQAPCGPQAFAHAAVAADSKVCSDIGRAILQQQGSPVDATIAALVCTSVVNPQSMGLGGGVIFTIYNVTTGKVEVINARETVPASHAPSLLDQCAQALPLGTGAQWIGVPGELRGYAEAHRRHGRLPWAQLFQPTIALLRGGHVVAPVLSRFLHNSILRPSLQASTLRQLFFNGTEPLRPQDPLPWPALATTLETVATEGVEVFYTGRLGQMLVEDIAKEGSQLTLQDLAKFQPEVVDALEVPLGDYTLYSPPPPAGGAILSFILNVLRGFNFSTESMARPEGRVNVYHHLVETLKFARGQRWRLGDPRSHPKLQNASRDLLGETLAQLIRQQIDGRGDHQLSHYSLAEAWGHGTGTSHVSVLGEDGSAVAATSTINTPFGAMVYSPRTGIILNNELLDLCERCPWGSGTTPSPVSGDRVGGAPGRCWPPVPGERSPSSMVPSILINKAQGSKLVIGGAGGELIISAVAQAIMSKLWLGFDLRAAIAAPILHVNSKGCVEYEPNFSQEVQRGLQDRGQNQTQRPFFLNVVQAVSQEGACVYAVSDLRKSGEAAGY

SEQ ID NO: 52 - DNA, 413 bp, Homo sapiens
tggtgccctccatcttgatcaacaaagcccaggggtcgaagctagtgattggcggggctggcggggagctcatcatctctgctgtggcccaggccatcatgagcaagctgtggcttggctttgacctgagagcggccattgcagcccccatcctgcatgtcaacagcaagggctgtgtggagtacgagcccaacttcagccaggtgaggctgaggtccgagctggatgcctagggcagagcccactccccaaatccgtgctgctcaaagccacctgggaggaactcagtcactgagattcttaggccagagacggtgtcttgctttgttgccctggctgttctcaaactcctggcctcaagtgatcctgccaccttggctcccaagtggctaagactgtaggtatgtgccacg

SEQ ID NO: 53 - DNA, 1644 bp, Homo sapiens
atggcccggggctacggggccacggtcagcctagtcctgctgggtctggggctggcgctggctgtcattgtgctggctgtggtcctctctcgacaccaggccccatgtggcccccaggcctttgcccacgctgctgttgccgccgactccaaggtctgctcggatattggacgagccatcctccagcagcagggctcacccgtggatgccaccatcgcggctctggtctgcaccagcgtcgtcaaccctcagagcatgggcctgggcggaggggtcatcttcaccatctacaatgtgacaacagggaaggtggaggtcatcaatgcccgggagacggtgccggccagccacgccccgagcctgctggaccagtgtgcacaggctctgccactgggcacaggggcccagtggatcggggtgcccggggagctccgtggctatgccgaggcccaccgccgccatggccgcctgccctgggcgcagctgttccagcccaccatcgcgctgctccgaggggggcatgtggtggcccctgtcctcagccgtttcctgcacaacagcatcctgcggccttccttgcaggcgtcaaccctgcgccagctcttcttcaacgggacagaacccctgaggcctcaggacccactcccatggcctgcactggccaccaccctggagaccgtggccacagagggcgtggaggtcttctacacggggaggctgggccagatgctggtggaggacattgccaaggaagggagccagctgacgctgcaggacctggccaagttccagcccgaggtggtggatgccctggaggtgcccctgggggactataccctgtactcaccaccgccgcctgcagggggtgccattctcagctttatcctcaacgtgctaagagggttcaacttctcaacagagtctatggccaggcctgaagggagggtgaacgtgtaccaccaccttgtagagacgctcaagtttgccagggggcagaggtggaggctgggggaccctcgaagccacccgaagctccagaatgcctcccgggacctgctgggggagaccctggcccagctcatccgccaacagatcgatggccggggggaccaccagctcagccactacagcttggccgaggcctggggccacgggacaggcacgtcccatgtgtctgtgctgggggaggatggcagcgccgtggctgccaccagcaccatcaacacaccctttggagcgatggtgtattcaccacggacaggcatcatcctcaacaacgagctcctggacttatgcgagcgatgcccctggggttccggcaccaccccctcacctgtgagtggagacagggtgggtggagctcccggaaggtgctggcccccagttccaggcgagcgttccccatcctccatggtgccctccatcttgatcaacaaagcccaggggtcgaagctagtgattggcggggctggcggggagctcatcatctctgctgtggcccaggccatcatgagcaagctgtggcttggctttgacctgagagcggccattgcagcccccatcctgcatgtcaacagcaagggctgtgtggagtacgagcccaacttcagccaggtgaggctgaggtccgagctggatgcctag

SEQ ID NO: 54 - protein, 547 aa, Homo sapiens
MARGYGATVSLVLLGLGLALAVIVLAVVLSRHQAPCGPQAFAHAAVAADSKVCSDIGRAILQQQGSPVDATIAALVCTSVVNPQSMGLGGGVIFTIYNVTTGKVEVINARETVPASHAPSLLDQCAQALPLGTGAQWIGVPGELRGYAEAHRRHGRLPWAQLFQPTIALLRGGHVVAPVLSRFLHNSILRPSLQASTLRQLFFNGTEPLRPQDPLPWPALATTLETVATEGVEVFYTGRLGQMLVEDIAKEGSQLTLQDLAKFQPEVVDALEVPLGDYTLYSPPPPAGGAILSFILNVLRGFNFSTESMARPEGRVNVYHHLVETLKFARGQRWRLGDPRSHPKLQNASRDLLGETLAQLIRQQIDGRGDHQLSHYSLAEAWGHGTGTSHVSVLGEDGSAVAATSTINTPFGAMVYSPRTGIILNNELLDLCERCPWGSGTTPSPVSGDRVGGAPGRCWPPVPGERSPSSMVPSILINKAQGSKLVIGGAGGELIISAVAQAIMSKLWLGFDLRAAIAAPILHVNSKGCVEYEPNFSQVRLRSELDA

SEQ ID NO: 55 - protein, 9 aa, Homo sapiens
VRLRSELDA

SEQ ID NO: 56 - DNA, 310 bp, Homo sapiens
attggtgccctccatcttgatcaacaaagcccaggggtcgaagctartgattggcggggctggcggggagctcatcatctctgctgtggcccaggccatcatgarcaagctgtggcttggctttgacctgagagcggccattgcagcccccatcctgcatgtcaacagcaagggctgtgtggagtacsascccaacttcasccagagacggtgtcttgctttgttgccctggctgttcatcaaactcctggcctcaagtgatcctgccaccttggctcccaagtggctaagactgtaggtatgtgccaca

SEQ ID NO: 57 - DNA, 1698 bp, Homo sapiens
atggcccggggctacggggccacggtcagcctagtcctgctgggtctggggctggcgctggctgtcattgtgctggctgtggtcctctctcgacaccaggccccatgtggcccccaggcctttgcccacgctgctgttgccgccgactccaaggtctgctcggatattggacgagccatcctccagcagcagggctcacccgtggatgccaccatcgcggctctggtctgcaccagcgtcgtcaaccctcagagcatgggcctgggcggaggggtcatcttcaccatctacaatgtgacaacagggaaggtggaggtcatcaatgcccgggagacggtgccggccagccacgccccgagcctgctggaccagtgtgcacaggctctgccactgggcacaggggcccagtggatcggggtgcccggggagctccgtggctatgccgaggcccaccgccgccatggccgcctgccctgggcgcagctgttccagcccaccatcgcgctgctccgaggggggcatgtggtggcccctgtcctcagccgtttcctgcacaacagcatcctgcggccttccttgcaggcgtcaaccctgcgccagctcttcttcaacgggacagaacccctgaggcctcaggacccactcccatggcctgcactggccaccaccctggagaccgtggccacagagggcgtggaggtcttctacacggggaggctgggccagatgctggtggaggacattgccaaggaagggagccagctgacgctgcaggacctggccaagttccagcccgaggtggtggatgccctggaggtgcccctgggggactataccctgtactcaccaccgccgcctgcagggggtgccattctcagctttatcctcaacgtgctaagagggttcaacttctcaacagagtctatggccaggcctgaagggagggtgaacgtgtaccaccaccttgtagagacgctcaagtttgccagggggcagaggtggaggctgggggaccctcgaagccacccgaagctccagaatgcctcccgggacctgctgggggagaccctggcccagctcatccgccaacagatcgatggccggggggaccaccagctcagccactacagcttggccgaggcctggggccacgggacaggcacgtcccatgtgtctgtgctgggggaggatggcagcgccgtggctgccaccagcaccatcaacacaccctttggagcgatggtgtattcaccacggacaggcatcatcctcaacaacgagctcctggacttatgcgagcgatgcccctggggttccggcaccaccccctcacctgtgagtggagacagggtgggtggagctcccggaaggtgctggcccccagttccaggcgagcgttccccatcctccatggtgccctccatcttgatcaacaaagcccaggggtcgaagctagtgattggcggggctggcggggagctcatcatctctgctgtggcccaggccatcatgagcaagctgtggcttggctttgacctgagagcggccattgcagcccccatcctgcatgtcaacagcaagggctgtgtggagtacgagcccaacttcagccagagacggtgtcttgctttgttgccctggctgttctcaaactcctggcctcaagtgatcctgccaccttggctcccaagtggctaa

SEQ ID NO: 58 - protein, 560 aa, Homo sapiens
MARGYGATVSLVLLGLGLALAVIVLAVVLSRHQAPCGPQAFAHAAVAADSKVCSDIGRAILQQQGSPVDATIAALVCTSVVNPQSMGLGGGVIFTIYNVTTGKVEVINARETVPASHAPSLLDQCAQALPLGTGAQWIGVPGELRGYAEAHRRHGRLPWAQLFQPTIALLRGGHVVAPVLSRFLHNSILRPSLQASTLRQLFFNGTEPLRPQDPLPWPALATTLETVATEGVEVFYTGRLGQMLVEDIAKEGSQLTLQDLAKFQPEVVDALEVPLGDYTLYSPPPPAGGAILSFILNVLRGFNFSTESMARPEGRVNVYHHLVETLKFARGQRWRLGDPRSHPKLQNASRDLLGETLAQLIRQQIDGRGDHQLSHYSLAEAWGHGTGTSHVSVLGEDGSAVAATSTINTPFGAMVYSPRTGIILNNELLDLCERCPWGSGTTPSPVSGDRVGGAPGRCWPPVPGERSPSSMVPSILINKAQGSKLVIGGAGGELIISAVAQAIMSKLWLGFDLRAAIAAPILHVNSKGCVEYEPNFSQRRCLALLPWLFSNSWPQVILPP

SEQ ID NO: 59 - protein, 22 aa, Homo sapiens
RRCLALLPWLFSNSWPQVILPP

SEQ ID NO: 60 - DNA, 2428 bp, Homo sapiens
gggccactccgtagtgtgcacttggtgagggcagcagctcgccacagctgccagccatctgtccattcacccatctgtccgtctggcagcccgctgttcagacctgtctgtctgtccgcccatctgtaagcccatctctgtcccattgtctatctgaccatctttctcttactgtcctctttgtctagctatctggcctatctgtcgatccatcttcgtgtctgtcttcagcccccacctgtttgtccatctgtccaattacctgtgactctgtgcatcttcttgtccattcatctgcccacccatccgtccctccgtctgcccaccagccgcccctctcctcctgggctgcagagccatggcccggggctacggggccacggtcagcctagtcctgctgggtctggggctggcgctggctgtcattgtgctggctgtggtcctctctcgacaccaggccccatgtggcccccaggcctttgcccacgctgctgttgccgccgactccaaggtctgctcggatattggacgagccatcctccagcagcagggctcacccgtggatgccaccatcgcggctctggtctgcaccagcgtcgtcaaccctcagagcatgggcctgggcggaggggtcatcttcaccatctacaatgtgacaacaggggcccagtggatcggggtgcccggggagctccgtggctatgccgaggcccaccgccgccatggccgcctgccctgggcgcagctgttccagcccaccatcgcgctgctccgaggggggcatgtggtggcccctgtcctcagccgtttcctgcacaacagcatcctgcggccttccttgcaggcgtcaaccctgcgccagctcttcttcaacgggacagaacccctgaggcctcaggacccactcccatggcctgcactggccaccaccctggagaccgtggccacagagggcgtggaggtcttctacacggggaggctgggccagatgctggtggaggacattgccaaggaagggagccagctgacgctgcaggacctggccaagttccagcccgaggtggtggatgccctggaggtgcccctgggggactataccctgtactcaccaccgccgcctgcagggggtgccattctcagctttatcctcaacgtgctaagagggttcaacttctcaacagagtctatggccaggcctgaagggagggtgaacgtgtaccaccaccttgtagagacgctcaagtttgccaaggggcagaggtggaggctgggggaccctcgaagccacccgaagctccagaatgcctcccgggacctgctgggggagaccctggcccagctcatccgccaacagatcgatggccggggggaccaccagctcagccactacagcttggccgaggcctggggccacgggacaggcacgtcccatgtgtcagtgctgggggaggatggcagcgccgtggctgccaccagcaccatctacacaccctttggagcgatggtgtattcaccacggacaggcatcatcctcaacaacgagctcctggacttatgcgagcgatgcccccggggttccggcaccaccccctcacctgtgagggagacagggtgggtggagctcccggaaggtgctggcccccagttccaggcgagcgttccccatcctccatggtgccctccatcttgatcaacaaagcccaggggtcgaagctagtgattggcggggctggcggggagctcatcatctctgctgtggcccaggccatcatgagcaagctgtggcttggctttgacctgagagcggccattgcagcccccatcctgcatgtcaacagcaagggctgtgtggagtacgagcccaacttcagccagagacggtgtcttgctttgttgccctggctgttctcaaactcctggcctcaagtgatcctgccaccttggctcccaagtggctaagactgtaggaggtgcagaggggactccaagaccgtggccagaaccagacccagaggcccttcttcctgaacgtggtccaggctgtgtcccaggagggggcctgtgtgtacgccgtctcggacctgaggaagagtggggaggccgcaggctactaagacactgctctgcccagagctgaagtctggccccaccatgagtcctgtgtccaggccggacatggctgggggaccaactactctggcaggatctggacccctggcaggggagtccagctgagagtggaagaggtggcggggaccagctgggcagatgagaggctgagcctcatccctaaccccctttcccagagcccctggtggtcctgaaccggcccctctatccctccgcaggcctcttgcctggggccactctcccaccctctcgatctgtatatcctccagtccaagattaaagaggcggactatgaaaa

SEQ ID NO: 61 - protein, 443 aa, Homo sapiens [sequence omitted]

SEQ ID NO: 62 - DNA, 149 bp, Homo sapiens
tccctgagtgtcagccccagaatggytcagtgacctgttttggaccggtgagctgctggcgggytcagagctgggtggaggggggcagcgagggggattgccagggacttggcaggatggcgagatgcagtagggtgtgctatctggat

SEQ ID NO: 63 - DNA, 4530 bp, Homo sapiens
aattctcgagctcgtcgaccggtcgacgagctcgagggtcgacgagctcgagggcgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagacgaaataaagacccaggggagaatgggtgttgtatggggaggcaagtgtggggggtccttctccacacccactttgtccatttgcaaatatattttggaaaac

SEQ ID NO: 64 - protein, 1255 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIVSAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERAKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 65 - DNA, 1836 bp, Homo sapiens
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggtgagctgctggcgggytcagagctgggtggaggggggcagcgagggggattgccagggacttggcaggatggcgagatgcagtagggtgtgctatctg

SEQ ID NO: 66 - protein, 612 aa, Homo sapiens
  MOD_RES (585)..(585): Variable amino acid
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPVSCWRXQSWVEGGSEGDCQGLGRMARCSRVCYL

SEQ ID NO: 67 - protein, 33 aa, Homo sapiens
  MOD_RES (6)..(6): Variable amino acid
VSCWRXQSWVEGGSEGDCQGLGRMARCSRVCYL

SEQ ID NO: 68 - DNA, 3383 bp, Homo sapiens
gctgcgcgcaggccgggaacacgaggccaagagccgcagccccagccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagatgccactgggcactgccctgccagtagtggccttcagggggctccttccggggctccggcctgttttccagagagagtggcagtagccccatggggcccggagctgctgtggcctccactggcatccgtgtttccaaaagtgcctttggcccaggctccacggcgacagttgggcccaaatcagaaaggagagagggggccaatgagggcagggcctcctgtgggctggaaaaccactgggtgcgtctcttgctggggtttgccctggaggtgaggtgagtgctcgagggaggggagtgctttctgccccatgcctccaactactgtatgcaggcctggctctctggtctaggccctttgggcaagaatgtccgtctacccggcttccaccaccctctggccctgggcttctgtaagcagacaggcagagggcctgcccctcccaccagccaagggtgccaggcctaactggggcactcagggcagtgtgttggaaattccactgagggggaaatcaggtgctgcggccgcctgggccctttcctccctcaagcccatctccacaacctcgagcctgggctctggtccactactgccccagaccaccctcaaagctggtcttcagaaatcaataatatgagtttttattttgtttttttttttttttttgtagtttattttggagtctagtatttcaataatttaagaatcagaagcactgacctttctacattttataacattattttgtatataat

SEQ ID NO: 69 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 70 - DNA, 2142 bp, Homo sapiens
atggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtgagtgctgggctcgcctttccttctgccttttgtgggagggaaagtggcctggtcactcttgacccatgggccattcctgaagggtag

SEQ ID NO: 71 - protein, 713 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEVSAGLAFPSAFCGRESGLVTLDPWAIPEG

SEQ ID NO: 72 - protein, 28 aa, Homo sapiens
SAGLAFPSAFCGRESGLVTLDPWAIPEG

SEQ ID NO: 73 - DNA, 222 bp, Homo sapiens
ggccgccagtgtgatggatatctgcagaattcgcccttcccagctgccccgtagtggggctcttactgttttcttttattccaagcccactatgcgagatttgtgtgtcagatgtaagctcgagagagcttgccttgaagggcgaattccagcacactgggcggccgttactagtggatccgagctcggtaccaagcttgatgcatagcttgactattctat

SEQ ID NO: 74 - DNA, 1543 bp, Homo sapiens
gctctcattaccttctgcccatcacttaataaatagccagccaattcatcaacattctggtacactgttggagagatgagacagtcacaccagctgcccctagtggggctcttactgttttcttttattccaagccaactatgcgagatttgtgaggtaagtgaagaaaactacatccgcctaaaacctctgttgaatacaatgatccagtcaaactataacaggggaaccagcgctgtcaatgttgtgttgtccctcaaacttgttggaatccagatccaaaccctgatgcaaaagatgatccaacaaatcaaatacaatgtgaaaagcagattgtcagatgtaagctcgggagagcttgccttgattatactggctttgggagtatgtcgtaacgctgaggaaaacttaatatatgattaccacctgatcgacaagctagaaaataaattccaagcagaaattgaaaatatggaagcacacaatggcactcccctgactaactactaccagctcagcctggacgttttggccttgtgtctgttcaatgggaactactcaaccgccgaagttgtcaaccacttcactcctgaaaataaaaactattattttggtagccagttctcagtagatactggtgcaatggctgtcctggctctgacctgtgtgaagaagagtctaataaatgggcagatcaaagcagatgaaggcagtttaaagaacatcagtatttatacaaagtcactggtagaaaagattctgtctgagaaaaaagaaaatggtctcattggaaacacatttagcacaggagaagccatgcaggccctctttgtatcatcagactattataatgaaaatgactggaattgccaacaaactctgaatacagtgctcacggaaatttctcaaggagcattcagcaatccaaacgctgcagcccaggtcttacctgccctgatgggaaagaccttcttggatattaacaaagactcttcttgcgtctctgcttcaggtaacttcaacatctccgctgatgagcctataactgtgacacctcctgactcacaatcatatatctccgtcaattactctgtgagaatcaatgaaacatatttcaccaatgtcactgtgctaaatggttctgtcttcctcagtgtgatggagaaagcccagaaaatgaatgatactatatttggtttcacaatggaggagcgctcatgggggccctatatcacctgtattcagggcctatgtgccaacaataatgacagaacctactgggaacttctgagtggaggcgaaccactgagccaaggagctggtagttacgttgtccgcaatggagaaaacttggaggttcgctggagcaaatactaataagcccaaactttcctcagctgcataaaatccatttgcagtggagttccatgtttattgtccttatgccttcttcttcatttatcccagtacgagcaggagagttaataacctccccttctctctctacatgttcaataaaagttgttgaaagattaacaactgt

SEQ ID NO: 75 - protein, 433 aa, Homo sapiens
MRQSHQLPLVGLLLFSFIPSQLCEICEVSEENYIRLKPLLNTMIQSNYNRGTSAVNVVLSLKLVGIQIQTLMQKMIQQIKYNVKSRLSDVSSGELALIILALGVCRNAEENLIYDYHLIDKLENKFQAEIENMEAHNGTPLTNYYQLSLDVLALCLFNGNYSTAEVVNHFTPENKNYYFGSQFSVDTGAMAVLALTCVKKSLINGQIKADEGSLKNISIYTKSLVEKILSEKKENGLIGNTFSTGEAMQALFVSSDYYNENDWNCQQTLNTVLTEISQGAFSNPNAAAQVLPALMGKTFLDINKDSSCVSASGNFNISADEPITVTPPDSQSYISVNYSVRINETYFTNVTVLNGSVFLSVMEKAQKMNDTIFGFTMEERSWGPYITCIQGLCANNNDRTYWELLSGGEPLSQGAGSYVVRNGENLEVRWSKY

SEQ ID NO: 76 - DNA, 1122 bp, Homo sapiens
atgagacagtcacaccagctgcccctagtggggctcttactgttttcttttattccaagccaactatgcgagatttgtgtgtcagatgtaagctcgggagagcttgccttgattatactggctttgggagtatgtcgtaacgctgaggaaaacttaatatatgattaccacctgatcgacaagctagaaaataaattccaagcagaaattgaaaatatggaagcacacaatggcactcccctgactaactactaccagctcagcctggacgttttggccttgtgtctgttcaatgggaactactcaaccgccgaagttgtcaaccacttcactcctgaaaataaaaactattattttggtagccagttctcagtagatactggtgcaatggctgtcctggctctgacctgtgtgaagaagagtctaataaatgggcagatcaaagcagatgaaggcagtttaaagaacatcagtatttatacaaagtcactggtagaaaagattctgtctgagaaaaaagaaaatggtctcattggaaacacatttagcacaggagaagccatgcaggccctctttgtatcatcagactattataatgaaaatgactggaattgccaacaaactctgaatacagtgctcacggaaatttctcaaggagcattcagcaatccaaacgctgcagcccaggtcttacctgccctgatgggaaagaccttcttggatattaacaaagactcttcttgcgtctctgcttcaggtaacttcaacatctccgctgatgagcctataactgtgacacctcctgactcacaatcatatatctccgtcaattactctgtgagaatcaatgaaacatatttcaccaatgtcactgtgctaaatggttctgtcttcctcagtgtgatggagaaagcccagaaaatgaatgatactatatttggtttcacaatggaggagcgctcatgggggccctatatcacctgtattcagggcctatgtgccaacaataatgacagaacctactgggaacttctgagtggaggcgaaccactgagccaaggagctggtagttacgttgtccgcaatggagaaaacttggaggttcgctggagcaaatactaa

SEQ ID NO: 77 - protein, 373 aa, Homo sapiens
MRQSHQLPLVGLLLFSFIPSQLCEICVSDVSSGELALIILALGVCRNAEENLIYDYHLIDKLENKFQAEIENMEAHNGTPLTNYYQLSLDVLALCLFNGNYSTAEVVNHFTPENKNYYFGSQFSVDTGAMAVLALTCVKKSLINGQIKADEGSLKNISIYTKSLVEKILSEKKENGLIGNTFSTGEAMQALFVSSDYYNENDWNCQQTLNTVLTEISQGAFSNPNAAAQVLPALMGKTFLDINKDSSCVSASGNFNISADEPITVTPPDSQSYISVNYSVRINETYFTNVTVLNGSVFLSVMEKAQKMNDTIFGFTMEERSWGPYITCIQGLCANNNDRTYWELLSGGEPLSQGAGSYVVRNGENLEVRWSKY

SEQ ID NO: 78 - protein, 13 aa, Homo sapiens
QLCEICVSDVSSG

SEQ ID NO: 79 - DNA, 1876 bp, Homo sapiens
ccaccaaccaaggcaactcagctgatgctgtaacaaccacagaaactgcgactagtggtcctacagtagctgcagctgataccactgaaactaatttccctgaaactgctagcaccacagcaaatacaccttctttcccaacagctacttcacctgctccccccataattagtacacatagttcctccacaattcctacacctgctccccccataattagtacacatagttcctccacaattcctatacctactgctgcagacagtgagtcaaccacaaatgtaaattcattagctacctctgacataatcaccgcttcatctccaaatgatggattaatcacaatggttccttctgaaacacaaagtaacaatgaaatgtcccccaccacagaagacaatcaatcatcagggcctcccactggcaccgctttattggagaccagcaccctaaacagcacaggtaaggacaattccctcaaagactccccaaggatggagagtacaattgtggagggagggaggtgggtatgttctggtggggtaggaaaatgggtctaaagaggcccaaggtttctttggaaatgaactgtgggcttgaaatctgtgtcacaatgtagtgacagcagaggccctaaaatcctcagtttccttctgttctcctttccttccctcccatcctacctcaagttatttgctagctgtgcagtattttttttaatgaacttgggctttgtgaaaggtagctctatagctttcctctccctaggggatttttagaaatagtttcagctgctctgagaagaccaggtccaaataagcacagactgtccagcaccactggtctagctctctggtctacctaaggaagaagtcagttctacccttgtaagatggtggctgatcacaatgtggaacttgagccaagtcatagaatttgagactgaggggtagaaagcaaaacaacagcagtgttcaaacaatcctaggcataaccagggccattgtccaaaggacagatgctttgggaacactaagagatgacaagtgacctcaagcagaagttaatgaaacaggaaccatttgcttcacttccccaagctcagcctataacagaaatcaactacgtttatcagtaaaggctaaatggccttgtgggccatgtggggtctctgttgtaactcctgcactctactattttaacatgaaagcagccacagttaacaaacaaaaggttggtcagatatgactcgtgaaccatagtttgccagcccttgatctagaagaatcgtcacactttagagcctaaagaatctagataaatcctatcattctcagatgggaaatctgggacccagtgagggccatgactcacccagggtcaaagatcaagtggatgtctccctacccttaacttctggtagcttcctcaatgttctttgatagatttaagaaatagatggttaagcaagtagaccccagaggctgtatctaagacctctttccccaatctttcatgtttggaggggccactctgaaggcgggatccaatgggacacagctgtcctgggatcaggaaagagaggttttctaagccatttctgcttcgccaggtgttccctcagagtcaggccatcttcctgtgttctggccctaccatgaacaaactgtggggcatggggcaagtcatttctctcttggcttcaacttagtgatctgcaataaggcgagactgaactaaaaagccccccaaatctcttctggctgtaacatcctgtgacttaatcaattcctggccatgaaacaagttaatgagtctgtccttcgttgctgaagagaaagcacctcagagttgtttgtctggtgtctccagaagg

SEQ ID NO: 80 - DNA, 1539 bp, Homo sapiens
atgaaagccatcattcatcttactcttcttgctctcctttctgtaaacacagccaccaaccaaggcaactcagctgatgctgtaacaaccacagaaactgcgactagtggtcctacagtagctgcagctgataccactgaaactaatttccctgaaactgctagcaccacagcaaatacaccttctttcccaacagctacttcacctgctccccccataattagtacacatagttcctccacaattcctacacctgctccccccataattagtacacatagttcctccacaattcctatacctactgctgcagacagtgagtcaaccacaaatgtaaattcattagctacctctgacataatcaccgcttcatctccaaatgatggattaatcacaatggttccttctgaaacacaaagtaacaatgaaatgtcccccaccacagaagacaatcaatcatcagggcctcccactggcaccgctttattggagaccagcaccctaaacagcacaggtcccagcaatccttgccaagatgatccctgtgcagataattcgttatgtgttaagctgcataatacaagtttttgcctgtgtttagaagggtattactacaactcttctacatgtaagaaaggaaaggtattccctgggaagatttcagtgacagtatcagaaacatttgacccagaagagaaacattccatggcctatcaagacttgcatagtgaaattactagcttgtttaaagatgtatttggcacatctgtttatggacagactgtaattcttactgtaagcacatctctgtcaccaagatctgaaatgcgtgctgatgacaagtttgttaatgtaacaatagtaacaattttggcagaaaccacaagtgacaatgagaagactgtgactgagaaaattaataaagcaattagaagtagctcaagcaactttctaaactatgatttgacccttcggtgtgattattatggctgtaaccagactgcggatgactgcctcaatggtttagcatgcgattgcaaatctgacctgcaaaggcctaacccacagagccctttctgcgttgcttccagtctcaagtgtcctgatgcctgcaacgcacagcacaagcaatgcttaataaagaagagtggtggggcccctgagtgtgcgtgcgtgcccggctaccaggaagatgctaatgggaactgccaaaagtgtgcatttggctacagtggactcgactgtaaggacaaatttcagctgatcctcactattgtgggcaccatcgctggcattgtcattctcagcatgataattgcattgattgtcacagcaagatcaaataacaaaacgaagcatattgaagaagagaacttgattgacgaagactttcaaaatctaaaactgcggtcgacaggcttcaccaatcttggagcagaagggagcgtctttcctaaggtcaggataacggcctccagagacagccagatgcaaaatccctattcaagacacagcagcatgccccgccctgactattag

SEQ ID NO: 81 - protein, 512 aa, Homo sapiens
MKAIIHLTLLALLSVNTATNQGNSADAVTTTETATSGPTVAAADTTETNFPETASTTANTPSFPTATSPAPPIISTHSSSTIPTPAPPIISTHSSSTIPIPTAADSESTTNVNSLATSDIITASSPNDGLITMVPSETQSNNEMSPTTEDNQSSGPPTGTALLETSTLNSTGPSNPCQDDPCADNSLCVKLHNTSFCLCLEGYYYNSSTCKKGKVFPGKISVTVSETFDPEEKHSMAYQDLHSEITSLFKDVFGTSVYGQTVILTVSTSLSPRSEMRADDKFVNVTIVTILAETTSDNEKTVTEKINKAIRSSSSNFLNYDLTLRCDYYGCNQTADDCLNGLACDCKSDLQRPNPQSPFCVASSLKCPDACNAQHKQCLIKKSGGAPECACVPGYQEDANGNCQKCAFGYSGLDCKDKFQLILTIVGTIAGIVILSMIIALIVTARSNNKTKHIEEENLIDEDFQNLKLRSTGFTNLGAEGSVFPKVRITASRDSQMQNPYSRHSSMPRPDY

SEQ ID NO: 82 - DNA, 4284 bp, Homo sapiens
ccacgcgtccgagcaagaacagctaaaatgaaagccatcattcatcttactcttcttgctctcctttctgtaaacacagccaccaaccaaggcaactcagctgatgctgtaacaaccacagaaactgcgactagtggtcctacagtagctgcagctgataccactgaaactaatttccctgaaactgctagcaccacagcaaatacaccttctttcccaacagctacttcacctgctccccccataattagtacacatagttcctccacaattcctacacctgctccccccataattagtacacatagttcctccacaattcctatacctactgctgcagacagtgagtcaaccacaaatgtaaattcattagctacctctgacataatcaccgcttcatctccaaatgatggattaatcacaatggttccttctgaaacacaaagtaacaatgaaatgtcccccaccacagaagacaatcaatcatcagggcctcccactggcaccgctttattggagaccagcaccctaaacagcacaggtaaggacaattccctcaaagactccccaaggatggagagtacaattgtggagggagggaggtgggtatgttctggtggggtaggaaaatgggtctaaagaggcccaaggtttctttggaaatgaactgtgggcttgaaatctgtgtcacaatgtagtgacagcagaggccctaaaatcctcagtttccttctgttctcctttccttccctcccatcctacctcaagttatttgctagctgtgcagtattttttttaatgaacttgggctttgtgaaaggtagctctatagctttcctctccctaggggatttttagaaatagtttcagctgctctgagaagaccaggtccaaataagcacagactgtccagcaccactggtctagctctctggtctacctaaggaagaagtcagttctacccttgtaagatggtggctgatcacaatgtggaacttgagccaagtcatagaatttgagactgaggggtagaaagcaaaacaacagcagtgttcaaacaatcctaggcataaccagggccattgtccaaaggacagatgctttgggaacactaagagatgacaagtgacctcaagcagaagttaatgaaacaggaaccatttgcttcacttccccaagctcagcctataacagaaatcaactacgtttatcagtaaaggctaaatggccttgtgggccatgtggggtctctgttgtaactcctgcactctactattttaacatgaaagcagccacagttaacaaacaaaaggttggtcagatatgactcgtgaaccatagtttgccagcccttgatctagaagaatcgtcacactttagagcctaaagaatctagataaatcctatcattctcagatgggaaatctgggacccagtgagggccatgactcacccagggtcaaagatcaagtggatgtctccctacccttaacttctggtagcttcctcaatgttctttgatagatttaagaaatagatggttaagcaagtagaccccagaggctgtatctaagacctctttccccaatctttcatgtttggaggggccactctgaaggcgggatccaatgggacacagctgtcctgggatcaggaaagagaggttttctaagccatttctgcttcgccaggtgttccctcagagtcaggccatcttcctgtgttctggccctaccatgaacaaactgtggggcatggggcaagtcatttctctcttggcttcaacttagtgatctgcaataaggcgagactgaactaaaaagccccccaaatctcttctggctgtaacatcctgtgacttaatcaattcctggccatgaaacaagttaatgagtctgtccttcgttgctgaagagaaagcacctcagagttgtttgtctggtgtctccagaagggtcccagcaatccttgccaagatgatccctgtgcagataattcgttatgtgttaagctgcataatacaagtttttgcctgtgtttagaagggtattactacaactcttctacatgtaagaaaggaaaggtattccctgggaagatttcagtgacagtatcagaaacatttgacccagaagagaaacattccatggcctatcaagacttgcatagtgaaattactagcttgtttaaagatgtatttggcacatctgtttatggacagactgtaattcttactgtaagcacatctctgtcaccaagatctgaaatgcgtgctgatgacaagtttgttaatgtaacaatagtaacaattttggcagaaaccacaagtgacaatgagaagactgtgactgagaaaattaataaagcaattagaagtagctcaagcaactttctaaactatgatttgacccttcggtgtgattattatggctgtaaccagactgcggatgactgcctcaatggtttagcatgcgattgcaaatctgacctgcaaaggcctaacccacagagccctttctgcgttgcttccagtctcaagtgtcctgatgcctgcaacgcacagcacaagcaatgcttaataaagaagagtggtggggcccctgagtgtgcgtgcgtgcccggctaccaggaagatgctaatgggaactgccaaaagtgtgcatttggctacagtggactcgactgtaaggacaaatttcagctgatcctcactattgtgggcaccatcgctggcattgtcattctcagcatgataattgcattgattgtcacagcaagatcaaataacaaaacgaagcatattgaagaagagaacttgattgacgaagactttcaaaatctaaaactgcggtcgacaggcttcaccaatcttggagcagaagggagcgtctttcctaaggtcaggataacggcctccagagacagccagatgcaaaatccctattcaagacacagcagcatgccccgccctgactattagaatcataagaatgtggaacccgccatggcccccaaccaatgtacaagctattatttagagtgtttagaaagactgatggagaagtgagcaccagtaaagatctggcctccggggtttttcttccatctgacatctgccagcctctctgaatggaagttgtgaatgtttgcaacgaatccagctcacttgctaaataagaatctatgacattaaatgtagtagatgctattagcgcttgtcagagaggtggttttcttcaatcagtacaaagtactgagacaatggttagggttgttttcttaattcttttcctggtagggcaacaagaaccatttccaatctagaggaaagctccccagcattgcttgctcctgggcaaacattgctcttgagttaagtgacctaattcccctgggagacatacgcatcaactgtggaggtccgaggggatgagaagggatacccaccacctttcaagggtcacaagctcactctctgacaagtcagaatagggacactgcttctatccctccaatggagagattctggcaacctttgaacagcccagagcttgcaacctagcctcacccaagaagactggaaagagacatatctctcagctttttcaggaggcgtgcctgggaatccaggaactttttgatgctaattagaaggcctggactaaaaatgtccactatggggtgcactctacagtttttgaaatgctaggaggcagaaggggcagagagtaaaaaacatgacctggtagaaggaagagaggcaaaggaaactgggtggggaggatcaattagagaggaggcacctgggatccaccttcttccttaggtcccctcctccatcagcaaaggagcacttctctaatcatgccctcccgaagactggctgggagaaggtttaaaaacaaaaaatccaggagtaagagccttaggtcagtttgaaattggagacaaactgtctggcaaagggtgcgagagggagcttgtgctcaggagtccagccgtccagcctcggggtgtaggtttctgaggtgtgccattggggcctcagccttctctggtgacagaggctcagctgtggccaccaacacacaaccacacacacacaaccacacacacaaatgggggcaaccacatccagtacaagcttttacaaatgttattagtgtccttttttatttctaatgccttgtcctcttaaaagttattttatttgttattattatttgttcttgactgttaattgtgaatggtaatgcaataaagtgcctttgttagatggtgaaa

SEQ ID NO: 83 - protein, 203 aa, Homo sapiens
MKAIIHLTLLALLSVNTATNQGNSADAVTTTETATSGPTVAAADTTETNFPETASTTANTPSFPTATSPAPPIISTHSSSTIPTPAPPIISTHSSSTIPIPTAADSESTTNVNSLATSDIITASSPNDGLITMVPSETQSNNEMSPTTEDNQSSGPPTGTALLETSTLNSTGKDNSLKDSPRMESTIVEGGRWVCSGGVGKWV

SEQ ID NO: 84 - protein, 32 aa, Homo sapiens
GKDNSLKDSPRMESTIVEGGRWVCSGGVGKWV

SEQ ID NO: 85 - DNA, 171 bp, Homo sapiens
gccagagtgctggaattcgcccatcctcatcgataaggtcatctccaccatcaccaacaacatccagcagatcattgagatcgaggacacctytgagacccttcgggacgggggtctgcaagaaaatgatgttcccacatagttggcagcacgtgaacagcaattgatccc

SEQ ID NO: 86 - DNA, 2691 bp, Homo sapiens
gcttgcccgtcggtcgctagctcgctcggtgcgcgtcgtcccgctccatggcgctcttcgtgcggctgctggctctcgccctggctctggccctgggccccgccgcgaccctggcgggtcccgccaagtcgccctaccagctggtgctgcagcacagcaggctccggggccgccagcacggccccaacgtgtgtgctgtgcagaaggttattggcactaataggaagtacttcaccaactgcaagcagtggtaccaaaggaaaatctgtggcaaatcaacagtcatcagctacgagtgctgtcctggatatgaaaaggtccctggggagaagggctgtccagcagccctaccactctcaaacctttacgagaccctgggagtcgttggatccaccaccactcagctgtacacggaccgcacggagaagctgaggcctgagatggaggggcccggcagcttcaccatcttcgcccctagcaacgaggcctgggcctccttgccagctgaagtgctggactccctggtcagcaatgtcaacattgagctgctcaatgccctccgctaccatatggtgggcaggcgagtcctgactgatgagctgaaacacggcatgaccctcacctctatgtaccagaattccaacatccagatccaccactatcctaatgggattgtaactgtgaactgtgcccggctcctgaaagccgaccaccatgcaaccaacggggtggtgcacctcatcgataaggtcatctccaccatcaccaacaacatccagcagatcattgagatcgaggacacctttgagacccttcgggctgctgtggctgcatcagggctcaacacgatgcttgaaggtaacggccagtacacgcttttggccccgaccaatgaggccttcgagaagatccctagtgagactttgaaccgtatcctgggcgacccagaagccctgagagacctgctgaacaaccacatcttgaagtcagctatgtgtgctgaagccatcgttgcggggctgtctgtagagaccctggagggcacgacactggaggtgggctgcagcggggacatgctcactatcaacgggaaggcgatcatctccaataaagacatcctagccaccaacggggtgatccactacattgatgagctactcatcccagactcagccaagacactatttgaattggctgcagagtctgatgtgtccacagccattgaccttttcagacaagccggcctcggcaatcatctctctggaagtgagcggttgaccctcctggctcccctgaattctgtattcaaagatggaacccctccaattgatgcccatacaaggaatttgcttcggaaccacataattaaagaccagctggcctctaagtatctgtaccatggacagaccctggaaactctgggcggcaaaaaactgagagtttttgtttatcgtaatagcctctgcattgagaacagctgcatcgcggcccacgacaagagggggaggtacgggaccctgttcacgatggaccgggtgctgacccccccaatggggactgtcatggatgtcctgaagggagacaatcgctttagcatgctggtagctgccatccagtctgcaggactgacggagaccctcaaccgggaaggagtctacacagtctttgctcccacaaatgaagccttccgagccctgccaccaagagaacggagcagactcttgggagatgccaaggaacttgccaacatcctgaaataccacattggtgatgaaatcctggttagcggaggcatcggggccctggtgcggctaaagtctctccaaggtgacaagctggaagtcagcttgaaaaacaatgtggtgagtgtcaacaaggagcctgttgccgagcctgacatcatggccacaaatggcgtggtccatgtcatcaccaatgttctgcagcctccagccaacagacctcaggaaagaggggatgaacttgcagactctgcgcttgagatcttcaaacaagcatcagcgttttccagggcttcccagaggtctgtgcgactagcccctgtctatcaaaagttattagagaggatgaagcattagcttgaagcactacaggaggaatgcaccacggcagctctccgccaatttctctcagatttccacagagactgtttgaatgttttcaaaaccaagtatcacactttaatgtacatgggccgcaccataatgagatgtgagccttgtgcatgtgggggaggagggagagagatgtactttttaaatcatgttccccctaaacatggctgttaacccactgcatgcagaaacttggatgtcactgcctgacattcacttccagagaggacctatcccaaatgtggaattgactgcctatgccaagtccctggaaaaggagcttcagtattgtggggctcataaaacatgaatcaagcaatccagcctcatgggaagtcctggcacagtttttgtaaagcccttgcacagctggagaaatggcatcattataagctatgagttgaaatgttctgtcaaatgtgtctcacatctacacgtggcttggaggcttttatggggccctgtccaggtagaaaagaaatggtatgtagagcttagatttccctattgtgacagagccatggtgtgtttgtaataataaaaccaaagaaacata

SEQ ID NO: 87 - protein, 683 aa, Homo sapiens
MALFVRLLALALALALGPAATLAGPAKSPYQLVLQHSRLRGRQHGPNVCAVQKVIGTNRKYFTNCKQWYQRKICGKSTVISYECCPGYEKVPGEKGCPAALPLSNLYETLGVVGSTTTQLYTDRTEKLRPEMEGPGSFTIFAPSNEAWASLPAEVLDSLVSNVNIELLNALRYHMVGRRVLTDELKHGMTLTSMYQNSNIQIHHYPNGIVTVNCARLLKADHHATNGVVHLIDKVISTITNNIQQIIEIEDTFETLRAAVAASGLNTMLEGNGQYTLLAPTNEAFEKIPSETLNRILGDPEALRDLLNNHILKSAMCAEAIVAGLSVETLEGTTLEVGCSGDMLTINGKAIISNKDILATNGVIHYIDELLIPDSAKTLFELAAESDVSTAIDLFRQAGLGNHLSGSERLTLLAPLNSVFKDGTPPIDAHTRNLLRNHIIKDQLASKYLYHGQTLETLGGKKLRVFVYRNSLCIENSCIAAHDKRGRYGTLFTMDRVLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANRPQERGDELADSALEIFKQASAFSRASQRSVRLAPVYQKLLERMKH

SEQ ID NO: 88 - DNA, 2596 bp, Homo sapiens
cttgcccgtcggtcgctagctcgctcggtgcgcgtcgtcccgctccatggcgctcttcgtgcggctgctggctctcgccctggctctggccctgggccccgccgcgaccctggcgggtcccgccaagtcgccctaccagctggtgctgcagcacagcaggctccggggccgccagcacggccccaacgtgtgtgctgtgcagaaggttattggcactaataggaagtacttcaccaactgcaagcagtggtaccaaaggaaaatctgtggcaaatcaacagtcatcagctacgagtgctgtcctggatatgaaaaggtccctggggagaagggctgtccagcagccctaccactctcaaacctttacgagaccctgggagtcgttggatccaccaccactcagctgtacacggaccgcacggagaagctgaggcctgagatggaggggcccggcagcttcaccatcttcgcccctagcaacgaggcctgggcctccttgccagctgaagtgctggactccctggtcagcaatgtcaacattgagctgctcaatgccctccgctaccatatggtgggcaggcgagtcctgactgatgagctgaaacacggcatgaccctcacctctatgtaccagaattccaacatccagatccaccactatcctaatgggattgtaactgtgaactgtgcccggctcctgaaagccgaccaccatgcaaccaacggggtggtgcacctcatcgataaggtcatctccaccatcaccaacaacatccagcagatcattgagatcgaggacacctttgagacccttcgggacgggggtctgcaagaaaatgatgttcccacatagttggcagcacgtgaacagcaattgatccctttgcatcacctcctcttactgtttagatttggctgctgtggctgcatcagggctcaacacgatgcttgaaggtaacggccagtacacgcttttggccccgaccaatgaggccttcgagaagatccctagtgagactttgaaccgtatcctgggcgacccagaagccctgagagacctgctgaacaaccacatcttgaagtcagctatgtgtgctgaagccatcgttgcggggctgtctgtagagaccctggagggcacgacactggaggtgggctgcagcggggacatgctcactatcaacgggaaggcgatcatctccaataaagacatcctagccaccaacggggtgatccactacattgatgagctactcatcccagactcagccaagacactatttgaattggctgcagagtctgatgtgtccacagccattgaccttttcagacaagccggcctcggcaatcatctctctggaagtgagcggttgaccctcctggctcccctgaattctgtattcaaagatggaacccctccaattgatgcccatacaaggaatttgcttcggaaccacataattaaagaccagctggcctctaagtatctgtaccatggacagaccctggaaactctgggcggcaaaaaactgagagtttttgtttatcgtaatagcctctgcattgagaacagctgcatcgcggcccacgacaagagggggaggtacgggaccctgttcacgatggaccgggtgctgacccccccaatggggactgtcatggatgtcctgaagggagacaatcgctttagcatgctggtagctgccatccagtctgcaggactgacggagaccctcaaccgggaaggagtctacacagtctttgctcccacaaatgaagccttccgagccctgccaccaagagaacggagcagactcttgggagatgccaaggaacttgccaacatcctgaaataccacattggtgatgaaatcctggttagcggaggcatcggggccctggtgcggctaaagtctctccaaggtgacaagctggaagtcagcttgaaaaacaatgtggtgagtgtcaacaaggagcctgttgccgagcctgacatcatggccacaaatggcgtggtccatgtcatcaccaatgttctgcagcctccagccaacagacctcaggaaagaggggatgaacttgcagactctgcgcttgagatcttcaaacaagcatcagcgttttccagggcttcccagaggtctgtgcgactagcccctgtctatcaaaagttattagagaggatgaagcattagcttgaagcactacaggaggaatgcaccacggcagctctccgccaatttctctcagatttccacagagactgtttgaatgttttcaaaaccaagtatcacactttaatgtacatgggccgcaccataatgagatgtgagccttgtgcatgtgggggaggagggagagagatgtactttttaaatcatgttccccctaaacatggctgttaacccactgcatgcagaaacttggatgtcactgcctgacattcacttccagagaggacctatcccaaatgtggaattgactgcctatgccaagtccctggaaaaggagcttcagtattgtggggctcataaaacatgaatcaagcaatccagcctcatgggaagtcctggcacagtttttgtaaagcccttgc

SEQ ID NO: 89 - protein, 268 aa, Homo sapiens [sequence omitted]

SEQ ID NO: 90 - protein, 11 aa, Homo sapiens
DGGLQENDVPT

SEQ ID NO: 91 - DNA, 177 bp, Homo sapiens
tagctgacgttctctcagcacgttcgctgcgaatgccggcctctgcgggagaagatgaagccggaaaggtgcggcgatgctgttccccggaggtaacccaccccttggaggagagagaccccgcacccggctcgtgtatttattaccgtcacactcttcagtgactcctgctggtac

SEQ ID NO: 92 - DNA, 1733 bp, Homo sapiens
ctgctgtctgcggaggaaactgcatcgacggacggccgcccagctacgggaggacctggagtggcactgggcgcccgacggaccatccccgggacccgcctgcccctcggcgccccgccccgccgggccgctccccgtcgggttccccagccacagccttacctacgggctcctgactccgcaaggcttccagaagatgctcgaaccaccggccggggcctcggggcagcagtgagggaggcgtccagccccccactcagctcttctcctcctgtgccaggggctccccgggggatgagcatggtggttttccctcggagccccctggctcgggacgtctgagaagatgccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggaggagacccaagggcagggggaagaggaggagagagaagcagagacccacagactgccacctgtgcggcgatgctgttccccggaggtaacccaccccttggaggagagagaccccgcacccggctcgtgtatttattaccgtcacactcttcagtgactcctgctggtacctgccctctatttattagccaactgtttccctgctgaatgcctcgctcccttcaagacgaggggcagggaaggacaggaccctcaggaattcagtgccttcaacaacgtgagagaaagagagaagccagccacagacccctgggagcttccgctttgaaagaagcaagacacgtggcctcgtgaggggcaagctaggccccagaggccctggaggtctccaggggcctgcagaaggaaagaagggggccctgctacctgttcttgggcctcaggctctgcacagtcaagcagcccttgctttcggagctcctgtccaaaagtagggatgcggatcctgctggggccgccacggcctggctggtgggaaggccggcagcgggcggaggggatccagccacttccccctcttcttctgaagatcagaacattcagctctggagaacagtggttgcctgggggcttttgccactccttgtcccccgtgatctcccctcacactttgccatttgcttgtactgggacattgttctttccggccaaggtgccaccaccctgccccccctaagagacacatacagagtgggccccgggctggagaaagagctgcctggatgagaaacagctcagccagtggggatgaggtcaccaggggaggagcctgtgcgtcccagctgaaggcagtggcaggggagcaggttccccaagggccctggcacccccacaagctgtccctgcagggccatctgactgccaagccagattctcttgaataaagtattctagtgtggaaaa

SEQ ID NO: 93 - DNA, 1645 bp, Homo sapiens
gggattcgggccgcccagctacgggaggacctggagtggcactgggcgcccgacggaccatccccgggacccgcctgcccctcggcgccccgccccgccgggccgctccccgtcgggttccccagccacagccttacctacgggctcctgactccgcaaggcttccagaagatgctcgaaccaccggccggggcctcggggcagcagtgagggaggcgtccagccccccactcagctcttctcctcctgtgccaggggctccccgggggatgagcatggtggttttccctcggagccccctggctcgggacgtctgagaagatgccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggtgcggcgatgctgttccccggaggtaacccaccccttggaggagagagaccccgcacccggctcgtgtatttattaccgtcacactcttcagtgactcctgctggtacctgccctctatttattagccaactgtttccctgctgaatgcctcgctcccttcaagacgaggggcagggaaggacaggaccctcaggaattcagtgccttcaacaacgtgagagaaagagagaagccagccacagacccctgggagcttccgctttgaaagaagcaagacacgtggcctcgtgaggggcaagctaggccccagaggccctggaggtctccaggggcctgcagaaggaaagaagggggccctgctacctgttcttgggcctcaggctctgcacagacaagcagcccttgctttcggagctcctgtccaaagtagggatgcggattctgctggggccgccacggcctggtggtgggaaggccggcagcgggcggaggggattcagccacttccccctcttcttctgaagatcagaacattcagctctggagaacagtggttgcctgggggcttttgccactccttgtcccccgtgatctcccctcacactttgccatttgcttgtactgggacattgttctttccggccgaggtgccaccaccctgcccccactaagagacacatacagagtgggccccgggctggagaaagagctgcctggatgagaaacagctcagccagtggggatgaggtcaccaggggaggagcctgtgcgtcccagctgaaggcagtggcaggggagcaggttccccaagggccctggcacccccacaagctgtccctgcagggccatctgactgccaagccagattctcttgaataaagtattctagtgtggaaacgc

SEQ ID NO: 94 - protein, 149 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERCGDAVPRR

SEQ ID NO: 95 - DNA, 1529 bp, Homo sapiens
  modified_base (1358)..(1358): a, c, t, g, unknown or other
gcacgagttgggaggtgtagcgcggctctgaacgcgctgagggccgttgagtgtcgcaggcggcgagggcgcgagtgaggagcagacccaggcatcgcgcgccgagaaggccgggcgtccccacactgaaggtccggaaaggcgacttccgggggctttggcacctggcggaccctcccggagcgtcggcacctgaacgcgaggcgctccattgcgcgtgcgcgttgaggggcttcccgcacctgatcgcgagaccccaacggctggtggcgtcgcctgcgcgtctcggctgagctggccatggcgcagctgtgcgggctgaggcggagccgggcgtttctcgccctgctgggatcgctgctcctctctggggtcctggcggccgaccgagaacgcagcatccacgacttctgcctggtgtcgaaggtggtgggcagatgccgggcctccatgcctaggtggtggtacaatgtcactgacggatcctgccagctgtttgtgtatgggggctgtgacggaaacagcaataattacctgaccaaggaggagtgcctcaagaaatgtgccactgtcacagagaatgccacgggtgacctggccaccagcaggaatgcagcggattcctctgtcccaagtgctcccagaaggcaggattctgaagaccactccagcgatatgttcaactatgaagaatactgcaccgccaacgcagtcactgggccttgccgtgcatccttcccacgctggtactttgacgtggagaggaactcctgcaataacttcatctatggaggctgccggggcaataagaacagctaccgctctgaggaggcctgcatgctccgctgcttccgccagcaggagaatcctcccctgccccttggctcaaaggtggtggttctggcggggctgttcgtgatggtgttgatcctcttcctgggagcctccatggtctacctgatccgggtggcacggaggaaccaggagcgtgccctgcgcaccgtctggagctccggagatgacaaggagcagctggtgaagaacacatatgtcctgtgaccgccctgtcgccaagaggactggggaagggaggggagactatgtgtgagctttttttaaatagagggattgactcggatttgagtgatcattagggctgaggtctgtttctctgggaggtaggacggctgcttcctggtctggcagggatgggtttgctttggaaatcctctaggaggctcctcctcgcatggcctgcagtctggcagcagccccgagttgtttcctcgctgatcgatttctttcctccaggtagagttttctttgcttatgttgaattccattgcctccttttctcnatcacagaagtgatgttggaatcgtttcttttgtttgtctgatttatggtttttttaagtataaacaaaagttttttattagcattctgaaagaaggaaagtaaaatgtacaagtttaataaaaaggggccttcccctttagaataaatttccagcatgttgctttcaaaa

SEQ ID NO: 96 - DNA, 639 bp, Homo sapiens
atggcgcagctgtgcgggctgaggcggagccgggcgtttctcgccctgctgggatcgctgctcctctctggggtcctggcggccgaccgagaacgcagcatccacgacttctgcctggtgtcgaaggtggtgggcagatgccgggcctccatgcctaggtggtggtacaatgtcactgacggatcctgccagctgtttgtgtatgggggctgtgacggaaacagcaataattacctgaccaaggaggagtgcctcaagaaatgtgccactgtcacagagaatgccacgggtgacctggccaccagcaggaatgcagcggattcctctgtcccaagtgctcccagaaggcaggattctgaagaccactccagcgatatgttcaactatgaaggtaaaactccaaagaggccagaatactgcaccgcaacgcagtcactgggccttgccgtgcatccttcccacgctggtactttgacgtggagaggaactcctgcaataacttcatctatggaggctgccggggcaataagaacagctaccgctctgaggaggcctgcatgctccgctgcttccgccagcaggagaatcctcccctgccccttggctcaaaggtggtggttctggcggggctgttcgtga

SEQ ID NO: 97 - protein, 212 aa, Homo sapiens
MAQLCGLRRSRAFLALLGSLLLSGVLAADRERSIHDFCLVSKVVGRCRASMPRWWYNVTDGSCQLFVYGGCDGNSNNYLTKEECLKKCATVTENATGDLATSRNAADSSVPSAPRRQDSEDHSSDMFNYEGKTPKRPEYCTATQSLGLAVHPSHAGTLTWRGTPAITSSMEAAGAIRTATALRRPACSAASASRRILPCPLAQRWWFWRGCS

SEQ ID NO: 98 - protein, 82 aa, Homo sapiens
GKTPKRPEYCTATQSLGLAVHPSHAGTLTWRGTPAITSSMEAAGAIRTATALRRPACSAASASRRILPCPLAQRWWFWRGCS

SEQ ID NO: 99 - protein, 16 aa, Homo sapiens
REKMKPERCGDAVPRR